(19) United States
(12) Patent Application Publication
(10) Pub. No.: US 2011/0098288 A1
(43) Pub. Date: Apr. 28, 2011

(54) SULFONAMIDES AS ZAP-70 INHIBITORS

(76) Inventors: Jeremy Major, Cambridge (GB); Richard John Harrison, Camberge (GB); Nigel Ramsden, Royston (GB); David Middlemiss, Hertsfordshire (GB); Ulrich Kruse, Dossenheim (DE); Gerard Drewes, Heidelberg (NL)

(21) Appl. No.: 12/922,163

(22) PCT Filed: Mar. 10, 2009
(86) PCT No.: PCT/EP2009/052789

§ 371 Date: Dec. 17, 2010

Related U.S. Application Data
(60) Provisional application No. 61/138,822, filed on Dec. 18, 2008.

(30) Foreign Application Priority Data
Mar. 11, 2008 (EP) 08152568.5

(51) Int. Cl. A61K 31/505; C07D 239/48; C07D 405/12; C07D 417/12; C07D 413/12; C07D 401/12; C07D 403/12; A61K 31/538; A61K 31/5377; A61K 31/506; A61P 37/00; A61P 29/00; A61P 37/08; A61P 19/02; A61P 25/00; A61P 17/06; A61P 1/00; A61P 1/04; A61P 11/06; A61P 11/00

(52) U.S. Cl. 514/230.5; 544/323; 544/324; 544/105; 544/122; 544/295; 514/275; 514/235.8; 514/252.14

(57) ABSTRACT

The invention relates to compounds of formula (I)

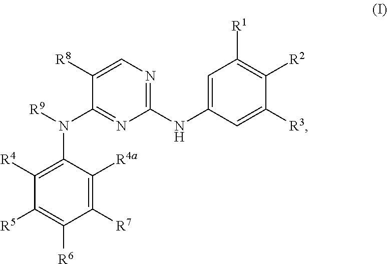

wherein R¹ to R⁹ and R^{4a} have the meaning as cited in the description and the claims. Said compounds are useful as inhibitors of ZAP-70 for the treatment or prophylaxis of immunological, inflammatory, autoimmune, allergic disorders, and immunologically-mediated diseases. The invention also relates to pharmaceutical compositions including said compounds, the preparation of such compounds as well as the use as medicaments.

### CROSS REFERENCE TO RELATED APPLICATIONS

The present application is a National Stage Application claiming the priority of co-pending PCT Application No. PCT/EP2009/052789 filed Mar. 10, 2009, which in turn, claims priority from European Patent Application No. 08152568.5 filed Mar. 11, 2008, and U.S. Provisional Application No. 61/138,822 filed Dec. 18, 2008. Applicants claim the benefits of 35 U.S.C. §120 as to the PCT application and priority under 35 U.S.C. §119 as to the said European Patent application and the said U.S. Provisional application, and the entire disclosures of all applications are incorporated herein by reference in their entireties.

The present invention relates to a novel class of kinase inhibitors, including pharmaceutically acceptable salts, prodrugs and metabolites thereof, which are useful for modulating protein kinase activity for modulating cellular activities such as signal transduction, proliferation, and cytokine secretion. More specifically the invention provides compounds which inhibit, regulate and/or modulate kinase activity, in particular ZAP-70 activity, and signal transduction pathways relating to cellular activities as mentioned above. Furthermore, the present invention relates to pharmaceutical compositions comprising said compounds, e.g. for the treatment of diseases such as immunological, inflammatory, autoimmune and allergic disorders, or immunologically-mediated diseases and processes for preparing said compounds.

Protein kinases participate in the signaling events which control the activation, growth and differentiation of cells in response to extracellular mediators or stimuli such as growth factors, cytokines or chemokines. In general, these kinases are classified in two groups, those that preferentially phosphorylate tyrosine residues and those that preferentially phosphorylate serine and/or threonine residues. The tyrosine kinases include membrane-spanning growth factor receptors such as the epidermal growth factor receptor (EGFR) and cytosolic non-receptor kinases such as Src, Syk or ZAP-70.

Inappropriately high protein kinase activity is involved in many diseases including inflammatory disorders and cancer. This can be caused either directly or indirectly by the failure of control mechanisms due to mutation, overexpression or inappropriate activation of the enzyme. In all of these instances, selective inhibition of the kinase is expected to have a beneficial effect.

Protein tyrosine kinases—both receptor tyrosine kinases and non-receptor kinases—are essential for the activation and proliferation of cells of the immune system. Among the earliest detectable events upon the immunoreceptor activation in mast cells, T cells and B cells is the stimulation of non-receptor tyrosine kinases. Immune receptors such as the high-affinity IgE receptor (FcεRI), T cell antigen receptor (TCR) and B cell receptor, consist of antigen-binding subunits and signal transducing subunits. The signal transducing chain contains one or more copies of immunoreceptor tyrosine-based activation motifs (ITAMSs). For TCR activation, ITAMS located in the CD3 molecule are phosphorylated by Lck and Fyn, two Src family tyrosine kinases, followed by recruitment and activation of ZAP-70, a member of the Syk family of tyrosine kinases. These activated tyrosine kinases then phosphorylate downstream adaptor molecules such as LAT (linker for activation of T cells) and SLP-76 (SH2 domain-containing leukocyte protein of 76 kDa). This step leads to the activation of multiple downstream signaling molecules such as inducible T cell kinase (ITK), PLCγ1 and PI3 kinase (Wong, 2005, Current Opinion in Pharmacology 5, 264-271; Schwartzberg et al. 2005, Nat. Rev. Immunology 5, 284-295).

ZAP-70 (zeta chain-associated protein of 70 kDa) belongs to the Syk family of tyrosine kinases and is associated with the zeta subunit of the T cell receptor (Chan et al., 1992, Cell 71(4): 649-662; Weiss, 1993, Cell 73, 209-212). ZAP-70 is primarily expressed in T cells and Natural Killer (NK) cells and plays an essential role in signaling through the TCR. The TCR-mediated activation of T cells is crucial for the immune response. Failure to adequately regulate T cell activation can lead to allergic and autoimmune diseases. Therefore ZAP-70 is considered as an attractive target for the development of immunosuppresive agents for T cell mediated diseases.

Several reports provided genetic evidence that ZAP-70 plays an important role in T cell activation. Mutations in ZAP-70 have been shown to be responsible for an autosomal recessive form of severe combined immunodeficiency syndrome (SCID) in humans (Elder 1998, Semin. Hematol. 35(4): 310-320). This SCID syndrome is characterized by the absence of peripheral CD8+ T cells and by the presence of circulating CD4+ T cells that do not respond to TCR-mediated stimuli in vitro. Targeted disruption of the ZAP-70 gene in mice leads to defects in thymic development and T cell activation (Negishi et al., 1995, Nature 376, 435-438). Inhibitors of ZAP-70 may therefore represent drugs useful for the treatment of diseases of the immune system (for example autoimmune diseases) or immunologically-mediated diseases (for example allograft transplant rejection and graft-versus-host disease).

A variety of approaches for the identification of selective ZAP-70 inhibitors have been reported. Vu suggested the structure-based design and synthesis of antagonists of the tandem Src-homology 2 (SH2) domains of ZAP-70 (Vu et al. 1999, 2000, Bioorg. Med. Chem. Letters 9, 3009-3014). Nishikawa screened a peptide library for the ability to bind to ZAP-70 and identified a peptide that inhibited ZAP-kinase activity by competing with protein substrates (Nishikawa et al., 2000, Molecular Cell 6, 969-974). Moffat used a ZAP-70 kinase assay with the non-physiological substrate polyGluTyr to identify ZAP-70 inhibitors (Moffat et al., 1999, Bioorg. Med. Chem. Letters 9, 3351-3356). In addition, the three-dimensional structure of the ZAP-70 kinase domain in complex with Staurosporine was reported and suggested as basis for the structure-based design of inhibitors (Jin et al., 2004, J. Biol. Chem. 279(41), 42818-42825).

In view of the above, there is a need for providing effective ZAP-70 inhibitors.

Inhibitors of FAK and/or ALK and/or ZAP-70 and/or IGF-IR are described in WO-A 2005/016894.

Thus, an object of the present invention is to provide a new class of compounds as kinase inhibitors, especially as ZAP-70 inhibitors, which may be effective in the treatment or prophylaxis of immunological, inflammatory, autoimmune, allergic disorders, immunologically-mediated diseases or other diseases or disorders associated with ZAP-70.

Accordingly, the present invention provides compounds of formula (I)

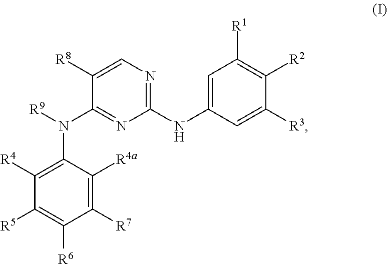

or a pharmaceutically acceptable salt, prodrug or metabolite thereof, wherein

R¹, R², R³ are independently selected from the group consisting of H; halogen; CN; C(O)OR¹⁰; OR¹⁰; C(O)R¹⁰; C(O)N(R¹⁰R^{10a}); S(O)₂N(R¹⁰R^{10a}); S(O)N(R¹⁰R^{10a}); S(O)₂R¹⁰; S(O)R¹⁰; N(R¹⁰)S(O)₂N(R^{10a}R^{10b}); SR¹⁰; N(R¹⁰R^{10a}); NO₂; OC(O)R¹⁰; N(R¹⁰)C(O)R^{10a}; N(R¹⁰)S(O)₂R^{10a}; N(R¹⁰)S(O)R^{10a}; N(R¹⁰)C(O)N(R^{10a}R^{10b}); N(R¹⁰)C(O)OR^{10a}; OC(O)N(R¹⁰R^{10a}); C₁₋₆ alkyl; C₂₋₆ alkenyl; C₂₋₆ alkynyl; and T, wherein C₁₋₆ alkyl; C₂₋₆ alkenyl; and C₂₋₆ alkynyl are optionally substituted with one or more R¹¹, which are the same or different;

Optionally, one of the pairs R¹/R² and R²/R³ is joined together with the phenyl ring to which it is attached to form a bicyclic ring T¹;

R¹⁰, R^{10a}, R^{10b} are independently selected from the group consisting of H; T; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹², which are the same or different;

R¹¹, R¹² are independently selected from the group consisting of T; halogen; CN; C(O)OR¹³; OR¹³; C(O)R¹³; C(O)N(R¹³R^{13a}); S(O)₂N(R¹³R^{13a}); S(O)N(R¹³R^{13a}); S(O)₂R¹³; S(O)R¹³; N(R¹³)S(O)₂N(R^{13a}R^{13b}); N(R¹³)S(O)N(R^{13a}R^{13b}); SR¹³; N(R¹³R^{13a}); NO₂; OC(O)R¹³; N(R¹³)C(O)R^{13a}; N(R¹³)S(O)₂R^{13a}; N(R¹³)S(O)R^{13a}; N(R¹³)C(O)N(R^{13a}R^{13b}); N(R¹³)C(O)OR^{13a}; OC(O)N(R¹³R^{13a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

R¹³, R^{13a}, R^{13b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

T is phenyl; C₃₋₇ cycloalkyl; or 4 to 7 membered heterocyclyl, wherein T is optionally substituted with one or more R¹⁴, which are the same or different;

T¹ is naphthyl; indenyl; indanyl; or 9 to 11 membered benzo-fused heterobicyclyl, wherein T¹ is optionally substituted with one or more R¹⁵, which are the same or different;

R¹⁴, R¹⁵ are independently selected from the group consisting of halogen; CN; C(O)OR¹⁶; OR¹⁶; oxo (═O), where the ring is at least partially saturated; C(O)R¹⁶; C(O)N(R¹⁶R^{16a}); S(O)₂N(R¹⁶R^{16a}); S(O)N(R¹⁶R^{16a}); S(O)₂R¹⁶; S(O)R¹⁶; N(R¹⁶)S(O)₂N(R^{16a}R^{16b}); N(R¹⁶)S(O)N(R^{16a}R^{16b}); SR¹⁶; N(R¹⁶R^{16a}); NO₂; OC(O)R¹⁶; N(R¹⁶)C(O)R^{16a}; N(R¹⁶)S(O)₂R^{16a}; N(R¹⁶)S(O)R^{16a}; N(R¹⁶)C(O)N(R^{16a}R^{16b}); N(R¹⁶)C(O)OR^{16a}; OC(O)N(R¹⁶R^{16a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

R¹⁶, R^{16a}, R^{16b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

R⁴, R⁵, R⁶, R⁷, R^{4a} are independently selected from the group consisting of H; X¹; halogen; CN; C(O)OR¹⁷; OR¹⁷; C(O)R¹⁷; C(O)N(R¹⁷R^{17a}); S(O)₂N(R¹⁷R^{17a}); S(O)N(R¹⁷R^{17a}); S(O)₂R¹⁷; S(O)R¹⁷; SR¹⁷; N(R¹⁷R^{17a}); NO₂; OC(O)R¹⁷; N(R¹⁷)C(O)R^{17a}; N(R¹⁷)S(O)₂R^{17a}; N(R¹⁷)S(O)R^{17a}; N(R¹⁷)C(O)N(R^{17a}R^{17b}); N(R¹⁷)C(O)OR^{17a}; OC(O)N(R¹⁷R^{17a}); C₁₋₆ alkyl; C₂₋₆ alkenyl; C₂₋₆ alkynyl; and T², wherein C₁₋₆ alkyl; C₂₋₆ alkenyl; and C₂₋₆ alkynyl are optionally substituted with one or more R¹⁸, which are the same or different and

wherein one of R⁴, R⁵, R⁶, R⁷, R^{4a} is X¹;

Optionally, one of the pairs R⁴/R⁵, R⁵/R⁶, R⁶/R⁷, R⁷/R^{4a} is joined together with the phenyl ring to which it is attached to form a bicyclic ring T³;

R¹⁷, R^{17a}, R^{17b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹⁹, which are the same or different;

R¹⁸, R¹⁹ are independently selected from the group consisting of T²; halogen; CN; C(O)OR²⁰; OR²⁰; C(O)R²⁰; C(O)N(R²⁰R^{20a}); S(O)₂N(R²⁰R^{20a}); S(O)N(R²⁰R^{20a}); S(O)₂R²⁰; S(O)R²⁰; N(R²⁰)S(O)₂N(R^{20a}R^{20b}); N(R²⁰)S(O)N(R^{20a}R^{20b}); SR²⁰; N(R²⁰R^{20a}); NO₂; OC(O)R²⁰; N(R²⁰)C(O)R^{20a}; N(R²⁰)S(O)₂R^{20a}; N(R²⁰)S(O)R^{20a}; N(R²⁰)C(O)N(R^{20a}R^{20b}); N(R²⁰)C(O)OR^{20a}; OC(O)N(R²⁰R^{20a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

R²⁰, R^{20a}, R^{20b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

T² is phenyl; C₃₋₇ cycloalkyl; or 4 to 7 membered heterocyclyl, wherein T² is optionally substituted with one or more R²¹, which are the same or different;

T³ is naphthyl; indenyl; indanyl; or 9 to 11 membered benzo-fused heterobicyclyl, wherein T³ is optionally substituted with one or more R²², which are the same or different;

R²¹, R²² are independently selected from the group consisting of halogen; CN; C(O)OR²³; OR²³; oxo (═O), where the ring is at least partially saturated; C(O)R²³; C(O)N(R²³R^{23a}); S(O)₂N(R²³R^{23a}); S(O)N(R²³R^{23a}); S(O)₂R²³; S(O)R²³; N(R²³)S(O)₂N(R^{23a}R^{23b}); N(R²³)S(O)N(R^{23a}R^{23b}); SR²³; N(R²³R^{23a}); NO₂; OC(O)R²³; N(R²³)C(O)R^{23a}; N(R²³)S(O)₂R^{23a}; N(R²³)S(O)R^{23a}; N(R²³)C(O)N(R^{23a}R^{23b}); N(R²³)C(O)OR^{23a}; OC(O)N(R²³R^{23a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

R²³, R^{23a}, R^{23b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

X¹ is N(R^{24a})S(O)₂R²⁴;

R⁹, R^{24a} are independently selected from the group consisting of H; C₁₋₄ alkyl; C₃₋₅ cycloalkyl; and C₃₋₅ cycloalkylmethyl, wherein C₁₋₄ alkyl; C₃₋₅ cycloalkyl and C₃₋₅ cycloalkylmethyl are optionally substituted with one or more halogen, which are the same or different;

R²⁴ is T⁴; C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R²⁵, which are the same or different;

R²⁵ is T⁴; halogen; CN; C(O)OR²⁶; OR²⁶; C(O)R²⁶; C(O)N(R²⁶R^{26a}); S(O)₂N(R²⁶R^{26a}); S(O)N(R²⁶R^{26a}); S(O)₂R²⁶; S(O)R²⁶; N(R²⁶)S(O)₂N(R^{26a}R^{26b}); N(R²⁶)S(O)N(R^{26a}R^{26b}); SR²⁶; N(R²⁶R^{26a}); NO₂; OC(O)R²⁶; N(R²⁶)C(O)R^{26a}; N(R²⁶)S(O)₂R^{26a}; N(R²⁶)S(O)R^{26a}; N(R²⁶)C(O)N(R^{26a}R^{26b}); N(R²⁶)C(O)OR^{26a}; OC(O)N(R²⁶R^{26a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more halogen, which are the same or different;

R²⁶, R^{26a}, R^{26b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

T⁴ is phenyl; C₃₋₇ cycloalkyl; or 4 to 7 membered heterocyclyl, wherein T⁴ is optionally substituted with one or more R²⁷, which are the same or different;

R²⁷ is halogen; CN; C(O)OR²⁸; OR²⁸; oxo (═O), where the ring is at least partially saturated; C(O)R²⁸; C(O)N(R²⁸R^{28a}); S(O)₂N(R²⁸R^{28a}); S(O)N(R²⁸R^{28a}); S(O)₂R²⁸; S(O)R²⁸; N(R²⁸)S(O)₂N(R^{28a}R^{28b}); N(R²⁸)S(O)N(R^{28a}R^{28b}); SR²⁸; N(R²⁸R^{28a}); NO₂; OC(O)R²⁸; N(R²⁸)C(O)R^{28a}; N(R²⁸)S(O)₂R^{28a}; N(R²⁸)S(O)R^{28a}; N(R²⁸)C(O)N(R^{28a}R^{28b}); N(R²⁸)C(O)OR^{28a}; OC(O)N(R²⁸R^{28a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more halogen, which are the same or different;

R²⁸, R^{28a}, R^{28b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;

R⁸ is H; F; Cl; Br; CN; C₁₋₄ alkyl; CH₂F; CHF₂; CF₃; OH; OCH₃; NO₂; NH₂; NHCH₃; N(CH₃)₂; or NO₂.

In case a variable or substituent can be selected from a group of different variants and such variable or substituent occurs more than once the respective variants can be the same or different.

Within the meaning of the present invention the terms are used as follows:

“Alkyl” means a straight-chain or branched saturated hydrocarbon chain. Each hydrogen of an alkyl carbon may be replaced by a substituent.

“Alkenyl” means a straight-chain or branched hydrocarbon chain, that contains at least one carbon-carbon double bond. Each hydrogen of an alkenyl carbon may be replaced by a substituent.

“Alkynyl” means a straight-chain or branched hydrocarbon chain, that contains at least one carbon-carbon triple bond. Each hydrogen of an alkynyl carbon may be replaced by a substituent.

“C₁₋₄ alkyl” means an alkyl chain having 1-4 carbon atoms, e.g. if present at the end of a molecule: methyl, ethyl, n-propyl, isopropyl, n-butyl, isobutyl, sec-butyl tert-butyl, or e.g. —CH₂—, —CH₂—CH₂—, —CH(CH₃)—, —C(CH₂)—, —CH₂—CH₂—CH₂—, —CH(C₂H₅)—, —C(CH₃)₂—, when two moieties of a molecule are linked by the alkyl group. Each hydrogen of a C₁₋₄ alkyl carbon may be replaced by a substituent.

“C₁₋₆ alkyl” means an alkyl chain having 1-6 carbon atoms, e.g. if present at the end of a molecule: C₁₋₄ alkyl, methyl, ethyl, n-propyl, isopropyl, n-butyl, isobutyl, sec-butyl, tert-butyl, n-pentyl, n-hexyl, or e.g. —CH₂—, —CH₂—CH₂—, —CH(CH₃)—, —C(CH₂)—, —CH₂—CH₂—CH₂—, —CH(C₂H₅)—, —C(CH₃)₂—, when two moieties of a molecule are linked by the alkyl group. Each hydrogen of a C₁₋₆ alkyl carbon may be replaced by a substituent.

“C₂₋₆ alkenyl” means an alkenyl chain having 2 to 6 carbon atoms, e.g. if present at the end of a molecule: —CH═CH₂, —CH═CH—CH₃, —CH₂—CH═CH₂, —CH═CH—CH₂—CH₃, —CH═CH—CH═CH₂, or e.g. —CH═CH—, when two moieties of a molecule are linked by the alkenyl group. Each hydrogen of a C₂₋₆ alkenyl carbon may be replaced by a substituent.

“C₂₋₆ alkynyl” means an alkynyl chain having 2 to 6 carbon atoms, e.g. if present at the end of a molecule: —C≡CH, —CH₂—C≡CH, CH₂—CH₂—C≡CH, CH₂—C≡C—CH₃, or e.g. —C≡C— when two moieties of a molecule are linked by the alkynyl group. Each hydrogen of a C₂₋₆ alkynyl carbon may be replaced by a substituent.

“C₃₋₇ cycloalkyl” or “C₃₋₇ cycloalkyl ring” means a cyclic alkyl chain having 3-7 carbon atoms, e.g. cyclopropyl, cyclobutyl, cyclopentyl, cyclohexyl, cyclohexenyl, cycloheptyl. Each hydrogen of a cycloalkyl carbon may be replaced by a substituent. Accordingly, “C₃₋₅ cycloalkyl” means a cycloalkyl having 3 to 5 carbon atoms.

“Halogen” means fluoro, chloro, bromo or iodo. It is generally preferred that halogen is fluoro or chloro.

“4 to 7 membered heterocyclyl” or “4 to 7 membered heterocycle” means a ring with 4, 5, 6 or 7 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 4 to 7 membered heterocycles are azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine or homopiperazine.

“9 to 11 membered heterobicyclyl” or “9 to 11 membered heterobicycle” means a heterocyclic system of two rings with 9 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for a 9 to 11 membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquino line, tetrahydroisoquinoline, dihydroisoquinoline, benzazepine, purine or pteridine. The term 9 to 11 membered heterobicycle also includes spiro structures of two rings like 1,4-dioxa-8-azaspiro[4.5]decane or bridged heterocycles like 8-aza-bicyclo[3.2.1]octane.

“benzofused” heterobicyclyl or “benzofused” heterobicycle means that one of the two rings of the bicycle is a benzene ring.

“5 to 6 membered aromatic heterocyclyl” or “5 to 6 membered aromatic heterocycle” means a heterocycle derived from cyclopentadienyl or benzene, where at least one carbon atom is replaced by a heteoatom selected from the group consisting of sulfur (including —S(O)—, —S(O)₂—), oxygen and nitrogen (including ═N(O)—). Examples for such heterocycles are furan, thiophene, pyrrole, imidazole, pyrazole, oxazole, isoxazole, thiazole, isothiazole, thiadiazole, pyranium, pyridine, pyridazine, pyrimidine, triazole, tetrazole.

Preferred compounds of formula (I) are those compounds in which one or more of the residues contained therein have the meanings given below, with all combinations of preferred substituent definitions being a subject of the present invention. With respect to all preferred compounds of the formula (I) the present invention also includes all tautomeric and stereoisomeric forms and mixtures thereof in all ratios, and their pharmaceutically acceptable salts.

In preferred embodiments of the present invention, the substituents mentioned below independently have the following meaning. Hence, one or more of these substituents can have the preferred or more preferred meanings given below.

Preferably, R^{4a} is X¹.

Preferably, none of the pairs R¹/R² and R²/R³ is joined together with the phenyl ring to which it is attached to form a bicyclic ring T¹ (T¹ is not present).

Preferably, R¹, R², R³ are independently selected from the group consisting of H; halogen; CN; OR¹⁰; NO₂; C(O)R¹⁰; Se; N(R¹⁰R^{10a}); T; and C₁₋₄ alkyl, wherein C₁₋₄ alkyl is optionally substituted with one or more halogen, which are the same or different. More preferably, R¹, R², R³ are independently selected from the group consisting of H; F; CN; NHR¹⁰; OR¹⁰; and C₁₋₄ alkyl.

Preferably, at least one of R¹, R², R³ is other than H.

Preferably one of R¹, R², R³ is selected from a group consisting of OR¹⁰; and NHR¹⁰, wherein R¹⁰ is methyl; ethyl; n-propyl; or iso-propyl and wherein methyl; ethyl; n-propyl; and iso-propyl are substituted with one substituent selected from group consisting of T; C(O)N(R¹³R^{13a}); N(R¹³R^{13a}); N(R¹³)C(O)R^{13a}; OH; and OCH₃.

Preferably R¹ and R² are OCH₃ and R³ is either H or OCH₃.

Preferably, R¹⁰, R^{10a} are independently selected from the group consisting of H; and C₁₋₄ alkyl, wherein C₁₋₄ alkyl is optionally substituted with one or more halogen, which are the same or different.

Preferably, R¹, R², R³ are independently selected from the group consisting of H; F; Cl; CN; OH; OCH₃; OCH₂CH₃; OCH₂F; OCHF₂; OCF₃; OCH₂CH₂F; OCH₂CHF₂; OCH₂CF₃; OCHFCH₂F; OCHFCHF₂; OCHFCF₃; OCF₂CH₂F; OCF₂CHF₂; OCF₂CF₃; NO₂; C(O)CH₃; SH; SCH₃; SCH₂F; SCHF₂; SCF₃; NH₂; NHCH₃; N(CH₃)₂; CH₃; CH₂CH₃; CH₂F; CHF₂; CF₃; CH₂CH₂F; CH₂CHF₂; CH₂CF₃; CHFCH₂F; CHFCHF₂; CHFCF₃; CF₂CH₂F; CF₂CHF₂; and CF₂CF₃. More preferably R¹, R², R³ are OCH₃.

Preferably, T is 4 to 7 membered heterocyclyl. More preferably, T is a 5 or 6 membered heterocycle, even more preferably a 5 membered heterocycle; even more preferably, imidazolyl; oxazolyl; thiazolyl; pyrazolyl; tetrazolyl; triazolyl; oxadiazolyl; morpholinyl; piperazinyl; pyrrolyl; pyrrolidinyl; or piperidinyl.

Preferably, T is unsubstituted or substituted with one or more R¹⁴, which are the same or different. Preferably, T is unsubstituted or substituted with one or two R¹⁴. Preferably, R¹⁴ is methyl; or oxo (═O), where the ring is at least partially saturated.

Preferably, R¹, R² are joined together with the phenyl ring to which they are attached to form 9 to 11 membered benzo-fused heterobicyclyl. More preferably, the bicyclic ring is selected from benzodioxane; benzothiazole; benzomorpholine; indole; indoline; indazole; benzoxazole; benzothiazole; or benzotriazole.

Preferably, each R¹⁵ is independently selected from the group consisting of F; Cl; oxo (═O), where the ring is at least partially saturated; OH; OCH₃; OCH₂CH₃; OCH₂F; OCHF₂; OCF₃; OCH₂CH₂F; OCH₂CHF₂; OCH₂CF₃; OCHFCH₂F; OCHFCHF₂; OCHFCF₃; OCF₂CH₂F; OCF₂CHF₂; OCF₂CF₃; NO₂; C(O)CH₃; SH; SCH₃; SCH₂F; SCHF₂; SCF₃; NH₂; NHCH₃; N(CH₃)₂; CH₃; CH₂CH₃; CH₂F; CHF₂; CF₃; CH₂CH₂F; CH₂CHF₂; CH₂CF₃; CHFCH₂F; CHFCHF₂; CHFCF₃; CF₂CH₂F; CF₂CHF₂; and CF₂CF₃.

Preferably, one of R⁴, R⁵, R⁶, R⁷, R^{4a} is X¹ and the others are selected from the group consisting of H; F; OH; OCH₃; OCH₂CH₃; OCH(CH₃)₂; CH₃; CH₂CH₃; and CH(CH₃)₂. Preferably, one of R⁴, R⁵, R⁶, R⁷, R^{4a} is X¹ and the others are selected from the group consisting of H; OH; OCH₃; OCH₂CH₃; and CH₃.

Preferably R⁶ is selected from the group consisting of H; OCH₃; OCH₂CH₃; and OCH(CH₃)₂. More preferably, R⁶ is OCH₃.

Preferably R⁷ is selected from the group consisting of H; CH₃; CH₂CH₃; and CH(CH₃)₂. More preferably, R⁷ is CH₃.

Preferably, R⁹; and R^{24a} are independently selected from the group consisting of H; CH₃; and CH₂CH₃. Preferably, R⁹; and R^{24a} are independently selected from the group consisting of H; and CH₃. More preferably, R⁹, R^{24a} are H.

Preferably, R²⁴ is C₁₋₄ alkyl. More preferably, R²⁴ is CH₃.

Preferably, R²⁴ is T⁴; or C₁₋₄ alkyl, wherein C₁₋₄ alkyl is substituted with one or more R²⁵, which are the same or different.

Preferably, T⁴ is phenyl; thiazolyl; imidazolyl; pyridyl; morpholinyl; piperazinyl, pyrrolidinyl; piperidinyl; or cyclopropyl.

Preferably, R²⁵ is F; Cl; OH; OCH₃; OCH₂CH₃; OCH₂F; OCHF₂; OCF₃; OCH₂CH₂F; OCH₂CHF₂; OCH₂CF₃; OCHFCH₂F; OCHFCHF₂; OCHFCF₃; OCF₂CH₂F; OCF₂CHF₂; OCF₂CF₃; NO₂; C(O)CH₃; SH; SCH₃; SCH₂F; SCHF₂; SCF₃; NH₂; NHCH₃; and N(CH₃)₂.

Preferably, R²⁴ is CH₂CF₃; T⁴; CH₂-T⁴; CH₂CH₂-T⁴; CH₂CH₂NHCH₃; or CH₂CH₂N(CH₃)₂.

Preferably, R²⁷ is CH₃.

Preferably, X¹ is NHS(O)₂CH₃; N(CH₃)S(O)₂CH₃; or N(CH₂CH₃)S(O)₂CH₃.

Preferably, R⁸ is H; F; Cl; Br; CN; CH₃; CH(CH₃)₂; CH₂F; CHF₂; CF₃; OH; OCH₃; NO₂; NH₂; NHCH₃; N(CH₃)₂; or NO₂. More preferably, R⁸H; CH₃; Br; Cl; or F. Even more preferably, R⁸ is Cl.

Compounds of formula (I) in which some or all of the above-mentioned groups have the preferred meanings are also an object of the present invention.

Further preferred compounds of the present invention are selected from the group consisting of
N-(2-(5-fluoro-2-(3,4,5-trimethoxyphenylamino)pyrimidin-4-ylamino)phenyl)methanesulfonamide;
N-(2-(5-fluoro-2-(4-fluorophenylamino)pyrimidin-4-ylamino)phenyl)methanesulfonamide;
N-(2-(2-(3,5-dimethylphenylamino)-5-fluoropyrimidin-4-ylamino)phenyl)methanesulfonamide;
N-(2-(2-(2,3-dihydrobenzo[b][1,4]dioxin-6-ylamino)-5-fluoropyrimidin-4-ylamino)phenyl)methanesulfonamide;
N-(2-(2-(3,5-difluorophenylamino)-5-fluoropyrimidin-4-ylamino)phenyl)methanesulfonamide;
N-(2-(5-fluoro-2-(3-fluorophenylamino)pyrimidin-4-ylamino)phenyl)methanesulfonamide;
N-(2-(2-(4-(dimethylamino)phenylamino)-5-fluoropyrimidin-4-ylamino)phenyl)methanesulfonamide;
N-(2-(2-(3,5-bis(trifluoromethyl)phenylamino)-5-fluoropyrimidin-4-ylamino)phenyl)methanesulfonamide;
N-(2-(5-fluoro-2-(4-(trifluoromethyl)phenylamino)pyrimidin-4-ylamino)phenyl)methanesulfonamide;
N-(2-(2-(4-chloro-3-methoxyphenylamino)-5-fluoropyrimidin-4-ylamino)phenyl)methanesulfonamide;
N-(2-(5-fluoro-2-(4-methyl-3-nitrophenylamino)pyrimidin-4-ylamino)phenyl)methanesulfonamide;
N-(2-(2-(3-chlorophenylamino)-5-fluoropyrimidin-4-ylamino)phenyl)methanesulfonamide;
N-(2-(2-(3-ethoxyphenylamino)-5-fluoropyrimidin-4-ylamino)phenyl)methanesulfonamide;
N-(2-(2-(3-acetylphenylamino)-5-fluoropyrimidin-4-ylamino)phenyl)methanesulfonamide;
N-(2-(5-fluoro-2-(3-(methylthio)phenylamino)pyrimidin-4-ylamino)phenyl)methanesulfonamide;
N-(2-(2-(benzo[d]thiazol-5-ylamino)-5-fluoropyrimidin-4-ylamino)phenyl)methanesulfonamide;
N-(2-(2-(3-(1H-pyrazol-1-yl)phenylamino)-5-fluoropyrimidin-4-ylamino)phenyl)methanesulfonamide;
N-(2-(5-fluoro-2-(2-methylbenzo[d]thiazol-5-ylamino)pyrimidin-4-ylamino)phenyl)methanesulfonamide;
N-(2-(2-(3-chloro-4-methoxyphenylamino)-5-fluoropyrimidin-4-ylamino)phenyl)methanesulfonamide;
N-(2-(5-fluoro-2-(4-methyl-3-oxo-3,4-dihydro-2H-benzo[b][1,4]oxazin-6-ylamino)pyrimidin-4-ylamino)phenyl)methanesulfonamide;
N-(2-(2-(3-(1H-1,2,4-triazol-1-yl)phenylamino)-5-fluoropyrimidin-4-ylamino)phenyl)methanesulfonamide;
N-(2-(2-(3-(3,5-dimethyl-1H-pyrazol-1-yl)phenylamino)-5-fluoropyrimidin-4-ylamino)phenyl)methanesulfonamide;
N-(2-(5-fluoro-2-(3-oxo-3,4-dihydro-2H-benzo[b][1,4]oxazin-7-ylamino)pyrimidin-4-ylamino)phenyl)methanesulfonamide;
N-(2-(5-fluoro-2-(3-(5-methyl-4H-1,2,4-triazol-3-yl)phenylamino)pyrimidin-4-ylamino)phenyl)methanesulfonamide;
N-(2-(5-fluoro-2-(4-methyl-3-oxo-3,4-dihydro-2H-benzo[b][1,4]oxazin-7-ylamino)pyrimidin-4-ylamino)phenyl)methanesulfonamide;
N-(2-(2-(3-(difluoromethoxy)phenylamino)-5-fluoropyrimidin-4-ylamino)phenyl)methanesulfonamide;
N-(2-(2-(4-(difluoromethoxy)phenylamino)-5-fluoropyrimidin-4-ylamino)phenyl)methanesulfonamide;
N-(2-(5-fluoro-2-(4-(trifluoromethoxy)phenylamino)pyrimidin-4-ylamino)phenyl)methanesulfonamide;
N-(2-(5-fluoro-2-(3-(trifluoromethoxy)phenylamino)pyrimidin-4-ylamino)phenyl)methanesulfonamide;
N-(2-(2-(4-chlorophenylamino)-5-fluoropyrimidin-4-ylamino)phenyl)methanesulfonamide;
N-(2-(5-fluoro-2-(3-(1,1,2,2-tetrafluoroethoxy)phenylamino)pyrimidin-4-ylamino)phenyl)methanesulfonamide;
N-(2-(2-(1H-indazol-6-ylamino)-5-fluoropyrimidin-4-ylamino)phenyl)methanesulfonamide;
N-(2-(2-(1H-benzo[d][1,2,3]triazol-6-ylamino)-5-fluoropyrimidin-4-ylamino)phenyl)methanesulfonamide;
N-(2-(5-fluoro-2-(phenylamino)pyrimidin-4-ylamino)phenyl)methanesulfonamide;
N-(2-(5-fluoro-2-(4-methoxyphenylamino)pyrimidin-4-ylamino)phenyl)methanesulfonamide;
N-(2-(2-(3,4-dimethoxyphenylamino)-5-fluoropyrimidin-4-ylamino)phenyl)methanesulfonamide;
N-(2-(2-(3,5-dimethoxyphenylamino)-5-fluoropyrimidin-4-ylamino)phenyl)methanesulfonamide;
N-(2-(5-fluoro-2-(3-methoxyphenylamino)pyrimidin-4-ylamino)phenyl)methanesulfonamide;
N-(4-(5-fluoro-2-(3,4,5-trimethoxyphenylamino)pyrimidin-4-ylamino)phenyl)thiophene-3-sulfonamide;
N-(4-(5-fluoro-2-(3,4,5-trimethoxyphenylamino)pyrimidin-4-ylamino)phenyl)thiophene-2-sulfonamide;
N-(4-(5-fluoro-2-(3,4,5-trimethoxyphenylamino)pyrimidin-4-ylamino)phenyl)pyridine-3-sulfonamide;
N-(4-(5-fluoro-2-(3,4,5-trimethoxyphenylamino)pyrimidin-4-ylamino)phenyl)-1-methyl-1H-imidazole-4-sulfonamide;
N-(4-(5-fluoro-2-(3,4,5-trimethoxyphenylamino)pyrimidin-4-ylamino)phenyl)-1-phenylmethanesulfonamide;
N-(4-(5-fluoro-2-(3,4,5-trimethoxyphenylamino)pyrimidin-4-ylamino)phenyl)benzenesulfonamide;
2,2,2-trifluoro-N-(4-(5-fluoro-2-(3,4,5-trimethoxyphenylamino)pyrimidin-4-ylamino)phenyl)ethanesulfonamide;
2,2,2-trifluoro-N-(2-(5-fluoro-2-(3,4,5-trimethoxyphenylamino)pyrimidin-4-ylamino)phenyl)ethanesulfonamide.
N-(2-(5-fluoro-2-(3,4,5-trimethoxyphenylamino)pyrimidin-4-ylamino)phenyl)benzenesulfonamide;
N-(2-(5-fluoro-2-(3,4,5-trimethoxyphenylamino)pyrimidin-4-ylamino)phenyl)-1-phenylmethanesulfonamide;
N-(2-(5-fluoro-2-(3,4,5-trimethoxyphenylamino)pyrimidin-4-ylamino)phenyl)thiopehene-3-sulfonamide;
N-(2-(5-fluoro-2-(3,4,5-trimethoxyphenylamino)pyrimidin-4-ylamino)phenyl)thiopehene-2-sulfonamide;
N-(2-(5-fluoro-2-(3,4,5-trimethoxyphenylamino)pyrimidin-4-ylamino)phenyl)-1-methyl-1H-imidazole-4-sulfonamide;
N-(2-(5-fluoro-2-(3,4,5-trimethoxyphenylamino)pyrimidin-4-ylamino)phenyl)pyridine-3-sulfonamide;
N-(2-(5-fluoro-2-(3,4,5-trimethoxyphenylamino)pyrimidin-4-ylamino)phenyl)pyridine-2-sulfonamide;
N-(3-(5-fluoro-2-(3,4,5-trimethoxyphenylamino)pyrimidin-4-ylamino)phenyl)thiophene-2-sulfonamide;
N-(3-(5-fluoro-2-(3,4,5-trimethoxyphenylamino)pyrimidin-4-ylamino)phenyl)thiophene-3-sulfonamide;
N-(3-(5-fluoro-2-(3,4,5-trimethoxyphenylamino)pyrimidin-4-ylamino)phenyl)-1-methyl-1H-imidazole-4-sulfonamide;
N-(3-(5-fluoro-2-(3,4,5-trimethoxyphenylamino)pyrimidin-4-ylamino)phenyl)pyridine-2-sulfonamide;
N-(3-(5-fluoro-2-(3,4,5-trimethoxyphenylamino)pyrimidin-4-ylamino)phenyl)pyridine-3-sulfonamide;
N-(3-(5-fluoro-2-(3,4,5-trimethoxyphenylamino)pyrimidin-4-ylamino)phenyl)benzenesulfonamide hydrochloride;
2,2,2-trifluoro-N-(3-(5-fluoro-2-(3,4,5-trimethoxyphenylamino)pyrimidin-4-ylamino)phenyl)ethanesulfonamide;
2-(Dimethylamino)-N-(2-(5-fluoro-2-(3,4,5-trimethoxyphenylamino)pyrimidin-4-ylamino)phenyl)ethanesulfonamide;
N-(2-(5-fluoro-2-(3,4,5-trimethoxyphenylamino)pyrimidin-4-ylamino)phenyl)-2-(methylamino)ethanesulfonamide;
N-(2-(5-fluoro-2-(3,4,5-trimethoxyphenylamino)pyrimidin-4-ylamino)-2-morpholinoethanesulfonamide;
N-(3-(5-fluoro-2-(3,4,5-trimethoxyphenylamino)pyrimidin-4-ylamino)phenyl)-2-(methylamino)ethanesulfonamide;
2-(Dimethylamino)-N-(3-(5-fluoro-2-(3,4,5-trimethoxyphenylamino)pyrimidin-4-ylamino)phenyl)ethanesulfonamide;
N-(2-(5-methyl-2-(3,4,5-trimethoxyphenylamino)pyrimidin-4-ylamino)phenyl)methanesulfonamide;
N-(2-(2-(3,5-dimethylphenylamino)-5-methylpyrimidin-4-ylamino)phenyl)methanesulfonamide;
N-(2-(5-nitro-2-(3,4,5-trimethoxyphenylamino)pyrimidin-4-ylamino)phenyl)methanesulfonamide;
N-(2-(2-(3,4,5-trimethoxyphenylamino)pyrimidin-4-ylamino)phenyl)methanesulfonamide;
N-(2-(5-bromo-2-(3,4,5-trimethoxyphenylamino)pyrimidin-4-ylamino)phenyl)methanesulfonamide;
N-(2-(5-fluoro-2-(3,4,5-trimethoxyphenylamino)pyrimidin-4-ylamino)phenyl)-N-methylmethanesulfonamide;
N-(2-(2-(3,5-dimethylphenylamino)-5-fluoropyrimidin-4-ylamino)phenyl)-N-methylmethanesulfonamide;
N-(2-(5-fluoro-2-(3-methoxy-4-methylphenylamino)pyrimidin-4-ylamino)phenyl)methanesulfonamide;
N-(2-(2-(3,4-dimethoxy-5-(2-(pyrrolidin-1-yl)ethoxy)phenylamino)-5-fluoropyrimidin-4-ylamino)phenyl)methanesulfonamide;
N-(2-(2-(3,5-dimethoxy-4-(2-(pyrrolidin-1-yl)ethoxy)phenylamino)-5-fluoropyrimidin-4-ylamino)phenyl)methanesulfonamide;
N-(2-(5-fluoro-2-(3-hydroxyphenylamino)pyrimidin-4-ylamino)phenyl)methanesulfonamide;
N-(2-(5-fluoro-2-(3-(2-morpholinoethoxy)phenylamino)pyrimidin-4-ylamino)phenyl)methanesulfonamide;
N-(2-(5-fluoro-2-(3-(2-hydroxyethoxy)phenylamino)pyrimidin-4-ylamino)phenyl)methanesulfonamide;
N-(2-(5-fluoro-2-(3-(2-(piperidin-1-yl)ethoxy)phenylamino)pyrimidin-4-ylamino)phenyl)methanesulfonamide formate;
3-(5-fluoro-4-(2-(methylsulfonamido)phenylamino)pyrimidin-2-ylamino)-N-methylbenzamide;
N,N-diethyl-3-(5-fluoro-4-(2-(methylsulfonamido)phenylamino)pyrimidin-2-ylamino)benzamide;
N-(2-(5-fluoro-2-(3-(pyrrolidine-1-carbonyl)phenylamino)pyrimidin-4-ylamino)phenyl)methanesulfonamide;
N-cyclopropyl-3-(5-fluoro-4-(2-(methylsulfonamido)phenylamino)pyrimidin-2-ylamino)benzamide;
3-(5-fluoro-4-(2-(methylsulfonamido)phenylamino)pyrimidin-2-ylamino)-N,N-dimethylbenzamide;
N-(2-(5-fluoro-2-(3-(pyrrolidin-1-yl)phenylamino)pyrimidin-4-ylamino)phenyl)methanesulfonamide;
N-(2-(5-fluoro-2-(4-(2-morholinoethoxy)phenylamino)pyrimidin-4-ylamino)phenyl)methanesulfonamide;
N-(2-(5-fluoro-2-(4-(piperidin-1-yl)ethoxy)phenylamino)pyrimidin-4-ylamino)phenyl)methanesulfonamide;
N-(2-(5-fluoro-2-(3-methoxy-4-(pyrrolidin-1-yl)phenylamino)pyrimidin-4-ylamino)phenyl)methanesulfonamide;
N-(2-(5-Fluoro-2-(4-(2-(4-methylpiperazin-1-yl)ethoxy)phenylamino)pyrimidin-4-ylamino)phenyl)methanesulfonamide;
N-(2-(5-fluoro-2-(4-(3-piperidin-1-yl)propoxy)phenylamino)pyrimidin-4-ylamino)phenyl)methanesulfonamide;
N-(2-(5-fluoro-2-(4-(3-(4-methylpiperazin-1-yl)propoxy)phenylamino)pyrimidin-4-ylamino)phenyl)methanesulfonamide;
N-(2-(5-fluoro-2-(4-(3-pyrrolidin-1-yl)propoxy)phenylamino)pyrimidin-4-ylamino)phenyl)methanesulfonamide;
N-(2-(5-fluoro-2-(4-(2-pyrrolidin-1-yl)ethylamino)phenylamino)pyrimidin-4-ylamino)phenyl)methanesulfonamide formate;
N-(2-(5-fluoro-2-(3-methoxy-5-(2-(pyrrolidin-1-yl)ethoxy)phenylamino)pyrimidin-4-ylamino)phenyl)methanesulfonamide formate salt;
N-(2-(5-fluoro-2-(3-(2-(pyrrolidin-1-yl)ethoxy)phenylamino)pyrimidin-4-ylamino)phenyl)methanesulfonamide;
N-(2-(5-fluoro-2-(4-(2-(pyrrolidin-1-yl)ethoxy)phenylamino)pyrimidin-4-ylamino)phenyl)methanesulfonamide;
N-(2-(5-fluoro-2-(3-((1-methylpiperidin-2-yl)methoxy)phenylamino)pyrimidin-4-ylamino)phenyl)methanesulfonamide;
N-(2-(5-fluoro-2-(3-((1-methylpiperidin-3-yl)methoxy)phenylamino)pyrimidin-4-ylamino)phenyl)methanesulfonamide;
2-(3-(5-fluoro-4-(2-(methylsulfonamido)phenylamino)pyrimidin-2-ylamino)phenoxy)acetic acid hydrochloride;
N,N-diethyl-2-(3-(5-fluoro-4-(2-(methylsulfonamido)phenylamino)pyrimidin-2-ylamino)phenoxy)acetamide 2,2,2-trifluoroacetate;
N-ethyl-2-(3-(5-fluoro-4-(2-(methylsulfonamido)phenylamino)pyrimidin-2-ylamino)phenoxy)acetamide;
N-(2-(5-bromo-2-(phenylamino)pyrimidin-4-ylamino)phenyl)methanesulfonamide hydrochloride;
N-(2-(5-bromo-2-(3-(difluoromethoxy)phenylamino)pyrimidin-4-ylamino)phenyl)methanesulfonamide;
N-(2-(5-bromo-2-(3-methoxy-4-methylphenylamino)pyrimidin-4-ylamino)phenyl)methanesulfonamide;
N-(2-(5-bromo-2-(3,5-dimethoxyphenylamino)pyrimidin-4-ylamino)phenyl)methanesulfonamide;
N-(2-(5-bromo-2-(4-methoxyphenylamino)pyrimidin-4-ylamino)phenyl)methanesulfonamide;
N-(2-(5-bromo-2-(3,4-dimethoxyphenylamino)pyrimidin-4-ylamino)phenyl)methanesulfonamide;
N-(2-(5-fluoro-2-(4-methoxyphenylamino)pyrimidin-4-ylamino)phenyl)-N-methylmethanesulfonamide;
N-(2-(2-(3,4-dimethoxyphenylamino)-5-fluoropyrimidin-4-ylamino)phenyl)-N-methylmethanesulfonamide;
N-(2-(5-fluoro-2-(4-(2-(piperidin-1-yl)ethoxy)phenylamino)pyrimidin-4-ylamino)phenyl)-N-methylmethanesulfonamide;
N-(2-(5-fluoro-2-(4-(2-morpholinoethoxy)phenylamino)pyrimidin-4-ylamino)phenyl)-N-methylmethanesulfonamide;
N-(2-(5-fluoro-2-(3-(2-(piperidin-1-yl)ethoxy)phenylamino)pyrimidin-4-ylamino)-6-methylphenyl)methanesulfonamide;
N-(2-(2-(3,5-dimethoxy-4-(2-(piperidin-1-yl)ethoxy)phenylamino)-5-fluoropyrimidin-4-ylamino)-6-methylphenyl)methanesulfonamide;
N-(2-(2-(3,4-dimethoxy-5-(2-(piperidin-1-yl)ethoxy)phenylamino)-5-fluoropyrimidin-4-ylamino)-6-methylphenyl)methanesulfonamide;
N-(2-(5-fluoro-2-(3-(2-methoxyethoxy)phenylamino)pyrimidin-4-ylamino)-6-methylphenyl)methanesulfonamide;
N-(2-(5-fluoro-2-(3,4,5-trimethoxyphenylamino)pyrimidin-4-ylamino)-6-methylphenyl)methanesulfonamide;
N-(2-(2-(3-ethoxy-4,5-dimethoxyphenylamino)-5-fluoropyrimidin-4-ylamino)-6-methylphenyl)methanesulfonamide;
N-(2-(5-fluoro-2-(3-isopropoxy-4,5-dimethoxyphenylamino)pyrimidin-4-ylamino)-6-methylphenyl)methanesulfonamide;
N-(2-(5-fluoro-2-(3-isobutoxy-4,5-dimethoxyphenylamino)pyrimidin-4-ylamino)-6-methylphenyl)methanesulfonamide;
N-(2-(2-(3-(cyclopropylmethoxy)-4,5-dimethoxyphenylamino)-5-fluoropyrimidin-4-ylamino)-6-methylphenyl)methanesulfonamide;
N-(2-(5-fluoro-2-(3-isopropoxyphenylamino)pyrimidin-4-ylamino)-6-methylphenyl)methanesulfonamide;
N-(2-(5-fluoro-2-(3-propoxyphenylamino)pyrimidin-4-ylamino)-6-methylphenyl)methanesulfonamide;
N-(2-(5-chloro-2-(3-(2-hydroxyethoxy)phenylamino)pyrimidin-4-ylamino)phenyl)methanesulfonamide;
N-(2-(5-chloro-2-(3-(2-(piperidin-1-yl)ethoxy)phenylamino)pyrimidin-4-ylamino)phenyl)methanesulfonamide;
N-(2-(5-chloro-2-(4-methoxy-3-(2-(piperidin-1-yl)ethoxy)phenylamino)pyrimidin-4-ylamino)phenyl)methanesulfonamide;
N-(2-(5-chloro-2-(3-(2-(diethylamino)ethoxy)phenylamino)pyrimidin-4-ylamino)phenyl)methanesulfonamide;
N-(2-(5-chloro-2-(4-(2-(diethylamino)ethoxy)phenylamino)pyrimidin-4-ylamino)phenyl)methanesulfonamide;
N-(2-(5-chloro-2-(3-(2-(piperidin-1-yl)ethoxy)phenylamino)pyrimidin-4-ylamino)-6-methylphenyl)methanesulfonamide;
N-(2-(5-chloro-2-(3-(piperidin-4-ylmethoxy)phenylamino)pyrimidin-4-ylamino)phenyl)methanesulfonamide;
N-(2-(5-chloro-2-(3-(2-(2-oxopyrrolidin-1-yl)ethoxy)phenylamino)pyrimidin-4-ylamino)phenyl)methanesulfonamide;
N-(2-(5-chloro-2-(3-(2-(pyrrolidin-2-yl)ethoxy)phenylamino)pyrimidin-4-ylamino)phenyl)methanesulfonamide;
N-(4-(5-chloro-4-(2-(methylsulfonamido)phenylamino)pyrimidin-2-ylamino)phenyl)-2-(pyrrolidin-1-yl)acetamide;
N-(2-(5-chloro-2-(3-(2-(piperazin-1-yl)ethoxy)phenylamino)pyrimidin-4-ylamino)phenyl)methanesulfonamide trifluoroacetate;
N-(2-(5-Chloro-2-(3-(3-piperidin-1-yl)propoxy)phenylamino)pyrimidin-4-ylamino)phenyl)methanesulfonamide;
N-(2-(5-chloro-2-(3-(3-methylpiperazin-1-yl)propoxy)phenylamino)pyrimidin-4-ylamino)phenyl)methanesulfonamide;
N-(2-(5-chloro-2-(4-(3-(4-methylpiperazin-1-yl)propoxy)phenylamino)pyrimidin-4-ylamino)phenyl)methanesulfonamide;
N-(2-(5-chloro-2-(3-isobutoxy-4,5-dimethoxyphenylamino)pyrimidin-4-ylamino)phenyl)methanesulfonamide;
N-(2-(5-chloro-2-(3,4,5-trimethoxyphenylamino)pyrimidin-4-ylamino)phenyl)methanesulfonamide hydrochloride;
N-(2-(5-chloro-2-(4-methoxyphenylamino)pyrimidin-4-ylamino)phenyl)methanesulfonamide hydrochloride;
N-(2-(5-chloro-2-(3-(2-(pyrrolidin-1-yl)ethoxy)phenylamino)pyrimidin-4-ylamino)phenyl)methanesulfonamide;
N-(2-(5-chloro-2-(3-methoxy-4-(2-(pyrrolidin-1-yl)ethoxy)phenylamino)pyrimidin-4-ylamino)phenyl)methanesulfonamide;
N-(2-(5-chloro-2-(3-methoxy-4-(2-(piperidin-1-yl)ethoxy)phenylamino)pyrimidin-4-ylamino)phenyl)methanesulfonamide hydrochloride;
Isopropyl 2-(4-(5-chloro-4-(2-(methylsulfonamido)phenylamino)pyrimidin-2-ylamino)-phenoxy)acetate hydrochloride;
Ethyl 2-(4-(5-fluoro-4-(2-(methylsulfonamido)phenylamino)pyrimidin-2-ylamino)phenoxy)acetate hydrochloride;
N-(2-(5-chloro-2-(3-((1-methylpiperidin-2-yl)methoxy)phenylamino)pyrimidin-4-ylamino)phenyl)methanesulfonamide;
N-(2-(5-chloro-2-(3-((1-methylpiperidin-3-yl)methoxy)phenylamino)pyrimidin-4-ylamino)phenyl)methanesulfonamide;
N-(2-(5-chloro-2-(3-((1,4-dimethylpiperazin-2-yl)methoxy)phenylamino)pyrimidin-4-ylamino)phenyl)methanesulfonamide;
2-(4-(5-chloro-4-(2-(methylsulfonamido)phenylamino)pyrimidin-2-ylamino)phenyl)acetic acid;
2-(3-(5-chloro-4-(2-(methylsulfonamido)phenylamino)pyrimidin-2-ylamino)phenoxy)acetic acid hydrochloride;
2-(3-(5-chloro-4-(2-(methylsulfonamido)phenylamino)pyrimidin-2-ylamino)phenoxy)-N,N-diethylacetamide;
2-(3-(5-chloro-4-(2-(methylsulfonamido)phenylamino)pyrimidin-2-ylamino)phenoxy)-N-ethylacetamide 2,2,2-trifluoroacetate;
N-(2-(5-chloro-2-(3,4,5-trimethoxyphenylamino)pyrimidin-4-ylamino)-5-methoxyphenyl)methanesulfonamide;
N-(2-(5-chloro-2-(3,4-dimethoxyphenylamino)pyrimidin-4-ylamino)-5-methoxyphenyl)methanesulfonamide;
N-ethyl-N-(2-(5-fluoro-2-(3,4,5-trimethoxyphenylamino)pyrimidin-4-ylamino)phenyl)methanesulfonamide;
N-(2-(2-(3,5-dimethylphenylamino)-5-fluoropyrimidin-4-ylamino)phenyl)-N-ethylmethanesulfonamide;
N-ethyl-N-(2-(5-fluoro-2-(4-methoxyphenylamino)pyrimidin-4-ylamino)phenyl)methanesulfonamide;
N-(2-(2-(3,4-dimethoxyphenylamino)-5-fluoropyrimidin-4-ylamino)phenyl)-N-ethylmethanesulfonamide;
N-ethyl-N-(2-(5-fluoro-2-(4-(2-(piperidin-1-yl)ethoxy)phenylamino)pyrimidin-4-ylamino)phenyl)methanesulfonamide;
N-ethyl-N-(2-(5-fluoro-2-(4-(2-morpholinoethoxy)phenylamino)pyrimidin-4-ylamino)phenyl)methanesulfonamide;
N-(2-(5-chloro-2-(3,4,5-trimethoxyphenylamino)pyrimidin-4-ylamino)-6-methylphenyl)methanesulfonamide;
N-(2-(5-fluoro-2-(3,4,5-trimethoxyphenylamino)pyrimidin-4-ylamino)-6-isopropylphenyl)methanesulfonamide;
N-(3-fluoro-6-(5-fluoro-2-(3,4,5-trimethoxyphenylamino)pyrimidin-4-ylamino)-2-methylphenyl)methanesulfonamide;
N-(6-(5-fluoro-2-(3,4,5-trimethoxyphenylamino)pyrimidin-4-ylamino)-3-methoxy-2-methylphenyl)methanesulfonamide;
N-(6-(5-chloro-2-(3,4,5-trimethoxyphenylamino)pyrimidin-4-ylamino)-2,3-dihydrobenzo[b][1,4]dioxin-5-yl)methanesulfonamide;
N-(6-(5-fluoro-2-(3,4,5-trimethoxyphenylamino)pyrimidin-4-ylamino)-2,3-dimethylphenyl)methanesulfonamide;
N-(2-ethoxy-6-(5-fluoro-2-(3,4,5-trimethoxyphenylamino)pyrimidin-4-ylamino)phenyl)methanesulfonamide;
N-(6-(5-fluoro-2-(3,4,5-trimethoxyphenylamino)pyrimidin-4-ylamino)-2,3-dihydrobenzo[b][1,4]dioxin-5-yl)methanesulfonamide;
N-(3-ethoxy-6-(5-fluoro-2-(3,4,5-trimethoxyphenylamino)pyrimidin-4-ylamino)-2-methylphenyl)methanesulfonamide;
N-(2-ethyl-6-(5-fluoro-2-(3,4,5-trimethoxyphenylamino)pyrimidin-4-ylamino)phenyl)methanesulfonamide;
N-(2-(5-Fluoro-2-(3,4,5-trimethoxyphenylamino)pyrimidin-4-ylamino)-5-methylphenyl)methanesulfonamide;
N-(2-(5-fluoro-2-(3,4,5-trimethoxyphenylamino)pyrimidin-4-ylamino)-5-morpholinophenyl)methanesulfonamide;
N-(2-(5-fluoro-2-(3,4,5-trimethoxyphenylamino)pyrimidin-4-ylamino)-6-methoxyphenyl)methanesulfonamide;
N-(2-(5-fluoro-2-(3,4,5-trimethoxyphenylamino)pyrimidin-4-ylamino)-5-methoxyphenyl)methanesulfonamide;
N-(4,5-difluoro-2-(5-fluoro-2-(3,4,5-trimethoxyphenylamino)pyrimidin-4-ylamino)phenyl)methanesulfonamide;
N-(5-ethoxy-2-(5-fluoro-2-(3,4,5-trimethoxyphenylamino)pyrimidin-4-ylamino)phenyl)methanesulfonamide;
N-(2-methyl-6-(2-(3,4,5-trimethoxyphenylamino)pyrimidin-4-ylamino)phenyl)methanesulfonamide;
N-(2-(2-(3-methoxy-5-(2-(piperidin-1-yl)ethoxy)phenylamino)pyrimidin-4-ylamino)-6-methylphenyl)methanesulfonamide formate salt;
N-(2-(5-bromo-2-(3,4,5-trimethoxyphenylamino)pyrimidin-4-ylamino)-5-methoxyphenyl)methanesulfonamide;
N-(2-(5-bromo-2-(3,4-dimethoxyphenylamino)pyrimidin-4-ylamino)-5-methoxyphenyl)methanesulfonamide;
N-(2-(2-(3,4-dimethoxyphenylamino)-5-fluoropyrimidin-4-ylamino)-5-methoxyphenyl)methanesulfonamide;
N-(2-(5-fluoro-2-(3,4,5-trimethoxyphenylamino)pyrimidin-4-ylamino)-6-methylphenyl)-N-methylmethanesulfonamide;
N-(2-(5-fluoro-2-(3,4,5-trimethoxyphenylamino)pyrimidin-4-ylamino)-5-isopropoxyphenyl)methanesulfonamide;
N-(2-fluoro-6-(5-fluoro-2-(3,4,5-trimethoxyphenylamino)pyrimidin-4-ylamino)phenyl)methanesulfonamide;
N-(2-chloro-6-(5-fluoro-2-(3,4,5-trimethoxyphenylamino)pyrimidin-4-ylamino)phenyl)methanesulfonamide;
N-(2-(5-fluoro-2-(3,4,5-trimethoxyphenylamino)pyrimidin-4-ylamino)-5-(3-(piperidin-1-yl)propoxy)phenyl)methanesulfonamide;
N-(2-(5-fluoro-2-(3,4,5-trimethoxyphenylamino)pyrimidin-4-ylamino)-4,6-dimethylphenyl)methanesulfonamide;
N-(6-(5-chloro-2-(3,4,5-trimethoxyphenylamino)pyrimidin-4-ylamino)-2-fluoro-3-methoxyphenyl)methanesulfonamide;
N-(2-(2-(3-(1H-tetrazol-1-yl)phenylamino)-5-fluoropyrimidin-4-ylamino)phenyl)methanesulfonamide;
N-(2-(5-fluoro-2-(3-(5-methyl-1,2,4-oxadiazol-3-yl)phenylamino)pyrimidin-4-ylamino)phenyl)methanesulfonamide;
N-(2-(2-(3-(1H-tetrazol-5-yl)phenylamino)-5-fluoropyrimidin-4-ylamino)phenyl)methanesulfonamide;
N-(2-(5-fluoro-2-(4-(3-methyl-1H-1,2,4-triazol-5-yl)phenylamino)pyrimidin-4-ylamino)phenyl)methanesulfonamide;
N-(2-(5-fluoro-2-(4-(2-methylthiazol-4-yl)phenylamino)pyrimidin-4-ylamino)phenyl)methanesulfonamide;
N-(2-(5-fluoro-2-(3-(oxazol-5-yl)phenylamino)pyrimidin-4-ylamino)phenyl)methanesulfonamide;
N-(2-(5-fluoro-2-(3-(5-methyl-1H-tetrazol-1-yl)phenylamino)pyrimidin-4-ylamino)phenyl)methanesulfonamide;
N-(2-(2-(4-(1H-tetrazol-1-yl)phenylamino)-5-fluoropyrimidin-4-ylamino)phenyl)methanesulfonamide;
N-(2-(2-(4-cyanophenylamino)-5-fluoropyrimidin-4-ylamino)phenyl)methanesulfonamide;
N-(2-(5-fluoro-2-(4-(5-methyl-1,2,4-oxadiazol-3-yl)phenylamino)pyrimidin-4-ylamino)phenyl)methanesulfonamide;
N-(2-(2-(4-(1H-1,2,4-triazol-1-yl)phenylamino)-5-fluoropyrimidin-4-ylamino)phenyl)methanesulfonamide;
N-(2-(5-fluoro-2-(4-(5-methyl-1H-tetrazol-1-yl)phenylamino)pyrimidin-4-ylamino)phenyl)methanesulfonamide;
N-(2-(2-(3-cyanophenylamino)-5-fluoropyrimidin-4-ylamino)phenyl)methanesulfonamide;
N-(2-(5-fluoro-2-(3-(1-methyl-1H-pyrazol-3-yl)phenylamino)pyrimidin-4-ylamino)phenyl)methanesulfonamide;
N-(2-(2-(3-(2,5-dimethyl-1H-pyrrol-1-yl)phenylamino)-5-fluoropyrimidin-4-ylamino)phenyl)methanesulfonamide;
N-(2-(2-(3-(1H-tetrazol-1-yl)phenylamino)-5-chloropyrimidin-4-ylamino)phenyl)methanesulfonamide;
N-(2-(5-chloro-2-(4-cyanophenylamino)pyrimidin-4-ylamino)phenyl)methanesulfonamide;
N-(2-(5-chloro-2-(3-cyanophenylamino)pyrimidin-4-ylamino)phenyl)methanesulfonamide;
N-(2-(2-(3-(1H-pyrazol-1-yl)phenylamino)-5-chloropyrimidin-4-ylamino)phenyl)methanesulfonamide;
N-(2-(2-(4-(1H-pyrazol-1-yl)phenylamino)-5-chloropyrimidin-4-ylamino)phenyl)methanesulfonamide;
N-(2-(5-chloro-2-(3-(5-methyl-1,2,4-oxadiazol-3-yl)phenylamino)pyrimidin-4-ylamino)phenyl)methanesulfonamide;
N-(2-(5-chloro-2-(3-(3,5-dimethyl-1H-pyrazol-1-yl)phenylamino)pyrimidin-4-ylamino)phenyl)methanesulfonamide;
N-(2-(5-chloro-2-(4-(3,5-dimethyl-1H-pyrazol-1-yl)phenylamino)pyrimidin-4-ylamino)phenyl)methanesulfonamide;
N-(2-(2-(3-(1H-1,2,4-triazol-1-yl)phenylamino)-5-chloropyrimidin-4-ylamino)phenyl)methanesulfonamide;
N-(2-(2-(4-(1H-1,2,4-triazol-1-yl)phenylamino)-5-chloropyrimidin-4-ylamino)phenyl)methanesulfonamide;
N-(2-(5-chloro-2-(3-(3-methyl-1H-1,2,4-triazol-5-yl)phenylamino)pyrimidin-4-ylamino)phenyl)methanesulfonamide;
N-(2-(2-(4-(1H-tetrazol-1-yl)phenylamino)-5-chloropyrimidin-4-ylamino)phenyl)methanesulfonamide;
N-(2-(5-bromo-2-(4-(piperidin-1-yl)phenylamino)pyrimidin-4-ylamino)phenyl)methanesulfonamide; and
N-(2-(2-(3-(1H-tetrazol-1-yl)phenylamino)-5-bromopyrimidin-4-ylamino)phenyl)methanesulfonamide.

Prodrugs of the compounds of the present invention are also within the scope of the present invention.

“Prodrug” means a derivative that is converted into a compound according to the present invention by a reaction with an enzyme, gastric acid or the like under a physiological condition in the living body, e.g. by oxidation, reduction, hydrolysis or the like, each of which is carried out enzymatically. Examples of a prodrug are compounds, wherein the amino group in a compound of the present invention is acylated, alkylated or phosphorylated to form, e.g., eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated. These compounds can be produced from compounds of the present invention according to well-known methods.

Metabolites of compounds of formula (I) are also within the scope of the present invention.

The term “metabolites” refers to all molecules derived from any of the compounds according to the present invention in a cell or organism, preferably mammal.

Preferably the term relates to molecules which differ from any molecule which is present in any such cell or organism under physiological conditions

The structure of the metabolites of the compounds according to the present invention will be obvious to any person skilled in the art, using the various appropriate methods.

Where tautomerism, like e.g. keto-enol tautomerism, of compounds of general formula (I) may occur, the individual forms, like e.g. the keto and enol form, are comprised separately and together as mixtures in any ratio. The same applies for stereoisomers, like e.g. enantiomers, cis/trans isomers, conformers and the like.

If desired, isomers can be separated by methods well known in the art, e.g. by liquid chromatography. The same applies for enantiomers by using e.g. chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of formula (I) may be obtained from stereoselective synthesis using optically pure starting materials.

The compounds of formula (I) may exist in crystalline or amorphous form. Furthermore, some of the crystalline forms of the compounds of formula (I) may exist as polymorphs, which are included within the scope of the present invention. Polymorphic forms of compounds of formula (I) may be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (ssNMR).

In case the compounds according to formula (I) contain one or more acidic or basic groups, the invention also comprises their corresponding pharmaceutically or toxicologically acceptable salts, in particular their pharmaceutically utilizable salts. Thus, the compounds of the formula (I) which contain acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the formula (I) which contain one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the formula (I) simultaneously contain acidic and basic groups in the molecule, the invention also includes, in addition to the salt forms mentioned, inner salts or betaines (zwitterions). The respective salts according to the formula (I) can be obtained by customary methods which are known to the person skilled in the art like, for example by contacting these with an organic or inorganic acid or base in a solvent or dispersant, or by anion exchange or cation exchange with other salts. The present invention also includes all salts of the compounds of the formula (I) which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means approved by a regulatory agency such as the EMEA (Europe) and/or the FDA (US) and/or any other national regulatory agency for use in animals, preferably in humans.

The present invention furthermore includes all solvates of the compounds according to the invention.

The present invention provides compounds of formula (I) as kinase inhibitors, especially as ZAP-70 inhibitors. The compounds of formula (I) may inhibit the kinase, optionally in addition to other kinases mentioned above without being limited by theory.

Accordingly, the compounds of the present invention are useful for the prevention or treatment of immunological, inflammatory, autoimmune, allergic disorders, or immunologically-mediated diseases, especially acute or chronic inflammation; rheumatoid arthritis; multiple sclerosis; psoriasis; Crohn's disease; ulcerative colitis; systemic lupus erythematosus; asthma; chronic obstructive pulmonary disease (COPD); allergic rhinitis; allograft transplant rejection; or graft-versus-host disease.

Without intending to be limited by theory, the compounds of the invention are useful for treating or preventing diseases that are mediated directly or indirectly by T cells. Indirect effects can be caused by influencing other types of immune cells, for example B cells.

Thus, another object of the present invention is a compound of the present invention or a pharmaceutically acceptable salt thereof for use as a medicament.

Another object of the present invention is a compound or a pharmaceutically acceptable salt thereof according to the present invention for use in a method of treating or preventing diseases and disorders associated with ZAP-70.

Yet another object of the present invention is the use of a compound of the present invention or a pharmaceutically acceptable salt thereof for the manufacture of a medicament for the treatment or prophylaxis of diseases and disorders associated with ZAP-70.

According to the present invention, the expression “ZAP-70” or “ZAP-70 kinase” means “zeta chain-associated protein of 70 kDa” (Chan et al, 1992, Cell 71(4):649-662). ZAP-70 associates with the zeta chain of the T cell receptor (TCR) and undergoes tyrosine phosphorylation following TCR stimulation. The ZAP-70 gene is located on human chromosome 2q12 and it is expressed in T cells and natural killer (NK) cells.

Yet another object of the present invention is a compound or a pharmaceutically acceptable salt thereof according to the present invention for use in a method of treating or preventing immunological, inflammatory, autoimmune, allergic disorders, or immunologically-mediated diseases.

Yet another object of the present invention is the use of a compound of the present invention or a pharmaceutically acceptable salt thereof for the manufacture of a medicament for the treatment or prophylaxis of immunological, inflammatory, autoimmune, allergic disorders, or immunologically-mediated diseases.

More specifically, preferred disorders are acute or chronic inflammation; rheumatoid arthritis; multiple sclerosis; psoriasis; Crohn's disease; ulcerative colitis; systemic lupus erythematosus; asthma; chronic obstructive pulmonary disease (COPD); allergic rhinitis; allograft transplant rejection; or graft-versus-host disease.

Quite more preferred are rheumatoid arthritis; multiple sclerosis; psoriasis; Crohn's disease; ulcerative colitis; systemic lupus erythematosus; allograft transplant rejection; or graft-versus-host disease.

Rheumatoid arthritis (RA) is a chronic progressive, debilitating inflammatory disease that affects approximately 1% of the world's population. RA is a symmetric polyarticular arthritis that primarily affects the small joints of the hands and feet. In addition to inflammation in the synovium, the joint lining, the aggressive front of tissue called pannus invades and destroys local articular structures (Firestein 2003, Nature 423:356-361).

Multiple sclerosis (MS) is an inflammatory and demyelating neurological disease. It has bee considered as an autoimmune disorder mediated by CD4+ type 1 T helper cells, but recent studies indicated a role of other immune cells (Hemmer et al., 2002, Nat. Rev. Neuroscience 3, 291-301).

Psoriasis is a chronic inflammatory dermatosis that affects approximately 2% of the population. It is characterized by red, scaly skin patches that are usually found on the scalp, elbows, and knees, and may be associated with severe arthritis. The lesions are caused by abnormal keratinocyte proliferation and infiltration of inflammatory cells into the dermis and epidermis (Schön et al., 2005, New Engl. J. Med. 352:1899-1912).

Inflammatory bowel disease (IBD) is characterized by a chronic relapsing intestinal inflammation. IBD is subdivided into Crohn's disease and ulcerative colitis phenotypes. Crohn disease involves most frequently the terminal ileum and colon, is transmural and discontinuous. In contrast, in ulcerative colitis, the inflammation is continuous and limited to rectal and colonic mucosal layers. In approximately 10% of cases confined to the rectum and colon, definitive classification of Crohn disease or ulcerative colitis cannot be made and are designated ‘indeterminate colitis.’ Both diseases include extraintestinal inflammation of the skin, eyes, or joints (Asakura et al., 2007, World J. Gastroenterol. 13(15):2145-2149).

Systemic lupus erythematosus (SLE) is a chronic inflammatory disease generated by T cell-mediated B-cell activation, which results in glomerulonephritis and renal failure. Human SLE is characterized at early stages by the expansion of long-lasting autoreactive CD4⁺ memory cells (D'Cruz et al., 2007, Lancet 369(9561):587-596).

Asthma is a complex syndrome with many clinical phenotypes in both adults and children. Its major characteristics include a variable degree of air flow obstruction, bronchial hyperresponsiveness, and airway inflammation (Busse and Lemanske, 2001, N. Engl. J. Med. 344:350-362).

Chronic obstructive pulmonary disease (COPD) is characterized by inflammation, airflow limitation that is not fully reversible, and a gradual loss of lung function. In COPD, chronic inhalation of irritants causes an abnormal inflammatory response, remodeling of the airways, and restriction of airflow in the lungs. The inhaled irritant is usually tobacco smoke, but occupational dust and environmental pollution are variably implicated (Shapiro 2005, N. Engl. J. Med. 352, 2016-2019).

Allergic rhinitis (also known as hay fever) is caused by pollens of specific seasonal plants and airborne chemicals or dust particles in patients who are allergic to these substances. It is characterized by sneezing, runny nose and itching eyes. The immune response to an allergen depends on an initial sensitization process and future exposure triggering the allergic response. This process involves several cell types and mediators of the immune system (Rosenwasser 2007, Allergy Asthma Proc. 28(1):10-15).

Immunologically-mediated diseases include rejection of transplanted organs or tissues (allografts) and graft-versus-host disease.

Allogaft transplant rejection includes, without limitation, acute and chronic allograft rejection following for example transplantation of kidney, heart, liver, lung, bone marrow, skin and cornea. It is known that T cells play a central role in the specific immune response of allograft rejection. Strategies to prevent T cell activation are expected to be useful for immunosuppression (Perico and Remuzzi, 1997. Drugs 54(4):533-570).

Graft-versus-host disease (GVDH) is a major complication in allogeneic bone marrow transplantation. GVDH is caused by donor T cells that recognize and react to recipient differences in the histocompatibility complex system, resulting in significant morbidity and mortality (Riddell and Appelbaum, 2007, PLoS Medicine 4 (7):1174-1177).

Another object of the present invention is a method for treating, controlling, delaying or preventing in a mammalian patient in need of the treatment of one or more conditions selected from the group consisting of diseases and disorders associated with ZAP-70, wherein the method comprises the administration to said patient a therapeutically effective amount of a compound according to present invention or a pharmaceutically acceptable salt thereof.

Yet another object is a method for treating, controlling, delaying or preventing in a mammalian patient in need of the treatment of one or more conditions selected from the group consisting of immunological, inflammatory, autoimmune, allergic disorders, and immunologically-mediated diseases, wherein the method comprises the administration to said patient a therapeutically effective amount of a compound according to the present invention or a pharmaceutically acceptable salt thereof.

More specifically the one or more conditions are selected from the group consisting of immunological, inflammatory, autoimmune, allergic disorders, or immunologically-mediated diseases, especially acute or chronic inflammation; rheumatoid arthritis; multiple sclerosis; psoriasis; Crohn's disease; ulcerative colitis; systemic lupus erythematosus; asthma; chronic obstructive pulmonary disease (COPD); allergic rhinitis; allograft transplant rejection; or graft-versus-host disease.

As used herein, the term “treating” or “treatment” is intended to refer to all processes, wherein there may be a slowing, interrupting, arresting, or stopping of the progression of a disease, but does not necessarily indicate a total elimination of all symptoms.

The compounds of the present invention may be further characterized by determining whether they have an effect on ZAP-70 activity, for example on its kinase activity (Isakov et al., 1996, J. Biol. Chem. 271(26), 15753-15761; Moffat et al., 1999, Bioorg. Med. Chem. Letters 9, 3351-3356).

The compounds of the present invention may also be characterized by measuring whether they have an effect on T cell receptor (TCR) signaling in a cell based assay using a T cell line or primary T cells. Cellular activation that is initiated by TCR signaling occurs as a result of a series of molecular events that include tyrosine phosphorylation of the CD3 zeta (CD3ζ) chain, recruitment of ZAP-70, phosphorylation of phospholipase gamma 1 (PLCγ1), inositol 1,4,5-triphosphate production, release of calcium stores from the endoplasmic reticulum to the cytoplasm, secretion of cytokines (for example Interleukin 2, IL-2), and cell proliferation.

The effect of compounds on tyrosine phosphorylation of PLCγ1 in Jurkat T cells following stimulation with anti-CD3 antibody can be examined by immunoprecipitation of PLCγ1 with an anti-PLCγ1 antibody and probing with an anti-phosphotyrosine specific antibody (e.g. antibody 4G10; Lin et al., 2004, Biochemistry 43, 11056-11062). Methods for measuring intracellular calcium release using fluorescent indicators for cytosolic calcium after TCR stimulation have been described (Meinl et al., 2000, J. Immunol. 165(7):3578-3583).

To evaluate the effect of compounds on the secretion of IL-2 T cells are stimulated with an anti-CD-3 antibody and incubated with various compound concentrations, then the concentration of IL-2 is measured in the cell-free media by an enzyme-linked immunosorbent assay (ELISA). A similar approach can be used to determine whether the compounds show activity in vivo. Mice are dosed with the compound of interest (e.g. by orally administration) followed by stimulation by intravenous injection of an anti-CD3 antibody. Serum is collected and the level of cytokines (e.g. IL-2) is measured in an ELISA (Lin et al., 2004, Biochemistry 43, 11056-11062).

The present invention provides pharmaceutical compositions comprising a compound of formula (I) or a pharmaceutically acceptable salt thereof as active ingredient together with a pharmaceutically acceptable carrier, optionally in combination with one or more other pharmaceutical compositions.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

A pharmaceutical composition of the present invention may comprise one or more additional compounds as active ingredients like one or more compounds of formula (I) not being the first compound in the composition or ZAP-70 inhibitors.

Other active ingredients for use in combination with other therapies for the treatment of immune, inflammatory, allergic disorders may include steroids, leukotriene antagonists, cyclosporine or rapamycin.

Other active ingredients include: immunosuppresants such as amtolmetin guacil, mizoribine and rimexolone; anti-TNFα agents such as etanercept, infliximab, Adalimumab, Anakinra, Abatacept, Rituximab; tyrosine kinase inhibitors such as leflunomide; kallikrein antagonists such as subreum; interleukin 11 agonists such as oprelvekin; interferon beta 1 agonists; hyaluronic acid agonists such as NRD-101 (Aventis); interleukin 1 receptor antagonists such as anakinra; CD8 antagonists such as amiprilose hydrochloride; beta amyloid precursor protein antagonists such as reumacon; matrix metalloprotease inhibitors such as cipemastat and other disease modifying anti-rheumatic drugs (DMARDs) such as methotrexate, sulphasalazine, cyclosporin A, hydroxychoroquine, auranofin, aurothioglucose, gold sodium thiomalate and penicillamine.

The individual compounds of such combinations may be administered either sequentially in separate pharmaceutical compositions as well as simultaneously in combined pharmaceutical compositions.

The pharmaceutical compositions of the present invention include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of formula (I) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally, for example, as liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula (I) may also be administered parenterally. Solutions or suspensions of these active compounds can be prepared in water suitably mixed with a surfactant such as hydroxypropyl-cellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of formula (I) are administered orally.

The effective dosage of active ingredient employed may vary depending on the particular compound employed, the mode of administration, the condition being treated and the severity of the condition being treated. Such dosage may be ascertained readily by a person skilled in the art.

A general route for the preparation of compounds according to present invention is outlined in Schemes 1 and 2.

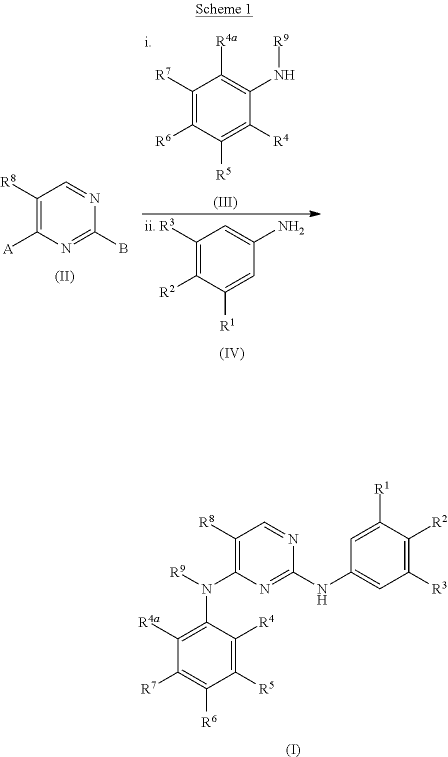

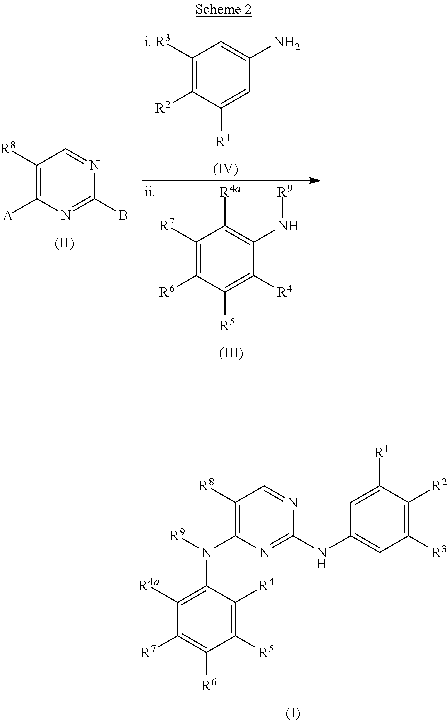

Compounds of formula (I) can be formed from compounds (II), (III) and (IV) by reacting (II) with (III) then reacting the resultant adduct with (IV) according to Scheme 1. Alternatively (I) may be formed by the reaction of (II) with (IV) then reacting the resultant adduct with (III) according to Scheme 2. The person skilled in the art would understand that the order of events would depend on the conditions of the reaction and the nature of (I), (II) and (III). Compounds (II), (III) and (IV) are either commercially available or can be made by those skilled in the art. A wide range of solvents are optionally employed for these reactions, including protic solvents such as alcohols, or polar aprotic solvents such as dimethylsulfoxide, DMF, acetonitrile, dioxane, THF. The reactions can optionally be promoted by the addition of a base which include but are not limited to amine bases such as triethylamine and DIPEA; or metal carbonates. The reactions can be optionally promoted by acids including mineral acids such as hydrogen chloride; organic acids and Lewis acids such as zinc (II) chloride. These reactions are typically performed between −78° C. and 160° C. depending on the nature of (I), (II) and (III). A and B are suitable leaving groups such as halogens, O—C₁₋₆ alkyl, N—C₁₋₆ alkyl, N(C₁₋₆ alkyl)₂, S—C₁₋₆ alkyl and SO₂—C₁₋₆ alkyl.

In one embodiment, a compound of formula (II) is reacted with a compound of formula (III) in the presence of an amine base, such as DIPEA; in a protic solvent, such as IPA; at a temperature above 20° C., such as 80° C. The adduct is isolated by means known to those skilled in the art, then reacted with a compound of formula (IV) in the presence of a mineral acid, such as hydrogen chloride; in a protic solvent such as IPA; at a temperature above 20° C., such as 80° C. to yield a compound of formula (I). In this embodiment it is conceivable that (I) is isolated in a salt form, such as a hydrochloride salt.

The sulfonamide functionality, X¹, can be introduced by reacting a compound of formula (I) wherein either R^{4a}, R⁴, R⁵, R⁶ or R⁷ is NHR^{24a} with a compound GS(O)₂R²⁴ wherein G is a suitable leaving group. Commonly G is chlorine. Alternatively this transformation may be effected on compound (III) or at an intermediate step in the synthesis of (I). The skilled person would recognise that a wide range of solvents may be employed to effect this process and that the addition of a base may be beneficial. In one embodiment, DCM is used as a solvent and triethylamine is used as a base. In another embodiment, pyridine is used as base and solvent. Compounds of formula GS(O)₂R²⁴ are either commercially available or can be prepared by those skilled in the art.

Accordingly, another aspect of the present invention is a method for the preparation of a compound of the present invention, comprising the steps of
(a) reacting a compound of formula (II)

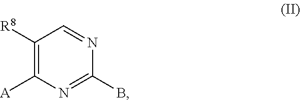

wherein R⁸ has the meaning as indicated above and A, B are suitable leaving groups with one of the compounds of formula (III) or (IV)

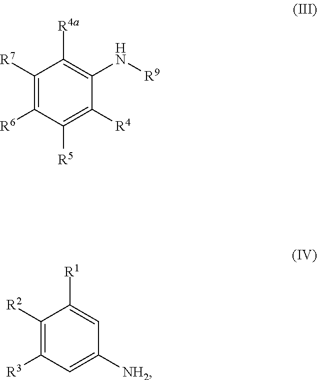

wherein R¹, R², R³, R⁴, R⁵, R⁶, R⁷, R⁹, R^{4a} have the meaning as indicated above provided that one of R⁴, R⁵, R⁶, R⁷, R^{4a} is NHR^{24a}; or N(R^{24a})S(O)₂R²⁴, wherein R²⁴, R^{24a} have the meaning as indicated above;
(b) further reacting the resulting product (IIa) from step (a) with the other compound of formula (III) or (IV); and
when one of R⁴, R⁵, R⁶, R⁷, R^{4a} is NHR^{24a},
reacting the compound of formula (III) before step (a), product (IIa) after step (a) or the resulting product from step (b) with a compound of formula GS(O)₂NR²⁴, wherein G is a suitable leaving group to yield compounds of formula (I).

It will be appreciated that novel intermediates described herein form another embodiment of the present invention.

### EXAMPLES

### Analytical Methods

NMR spectra were obtained on a Bruker dpx400. LCMS was carried out on an Agilent 1100 using a ZORBAX® SB-C18, 4.6×150 mm, 5 microns or ZORBAX® SB-C18, 4.6×75 mm, 3.5 micron column. Column flow was 1 mL/min and solvents used were water and acetonitrile (0.1% formic acid) with an injection volume of 10 uL. Wavelengths were 254 and 210 nm. Methods are described below.

### Method A

Column: Gemini C18, 3×30 mm, 3 microns Flow: 1.2 mL/min. Gradient: Table 1

TABLE 1

| Time (min) | Water | Acetonitrile |
|---|---|---|
| 0 | 95 | 5 |
| 3 | 5 | 95 |
| 4.5 | 5 | 95 |
| 4.6 | 95 | 5 |

| | |
|---|---|
| 5.00 | STOP |

### Method B

Column: ZORBAX® SB-C18, 4.6×150 mm, 5 microns. Flow: 1 mL/min. Gradient: Table 2

TABLE 2

| Time (min) | Water | Acetonitrile |
|---|---|---|
| 0 | 95 | 5 |
| 11 | 5 | 95 |
| 13 | 5 | 95 |
| 13.01 | 95 | 5 |

| | |
|---|---|
| 14.00 | STOP |

### Method C

As Method A but with 0.1% ammonium hydroxide instead of 0.1% formic acid.

### Abbreviations

TABLE 3

| | |
|---|---|
| DCM | dichloromethane |
| THF | tetrahydrofuran |
| IPA | iso-propyl alcohol |
| petrol | petroleum ether, boiling point 40-60° C. |
| DMF | N, N-dimethylformamide |
| TFA | trifluoroacetic acid |
| DIPEA | di-iso-propylethylamine |
| Me | methyl |
| Et | ethyl |
| ⁱPr | iso-propyl |
| Ph | phenyl |
| Bn | benzyl |
| Boc | tert-butyloxycarbonyl |
| h | hour |
| min | minute |
| M | molar |
| sat. | saturated |
| (aq) | aqueous |
| NMR | nuclear magnetic resonance |
| MeOD | deuterated methanol (d₄-methanol) |
| s | singlet |
| d | doublet |
| dd | doublet doublet |
| td | triplet doublet |
| br | broad |
| t | triplet |
| m | multiplet |
| ES+ | electrospray positive ionisation |
| RT | retention time |

### Intermediate 1a

### N-(2-(2-chloro-5-fluoropyrimidin-4-ylamino)phenyl)methanesulfonamide

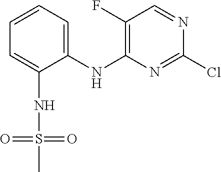

### Step (i)

### N1-(2-chloro-5-fluoropyrimidin-4-yl)benzene-1,2-diamine

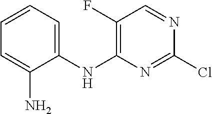

A mixture of 2,4-dichloro-5-fluoropyrimidine (10.0 g, 0.06 mol), o-phenylenediamine (7.1 g, 0.066 mol) and DIPEA (20.8 mL, 0.12 mol) in n-butanol (80 mL) was stirred at 110° C. for 16 h then concentrated in vacuo and slurried with 0.1 M hydrochloric acid (20 mL). The solid was collected at the pump, washed with water (2×20 mL), n-butanol (30 mL and diethyl ether (2×30 mL), then dried under vacuum to afford N1-(2-chloro-5-fluoropyrimidin-4-yl)benzene-1,2-diamine as a colourless powder (10.8 g, 71%). ¹H NMR (d₆-DMSO) δ 9.31 (br s, 1H), 8.18 (d, 1H), 6.99-7.03 (m, 2H), 6.74-6.76 (m, 1H), 6.54-6.58 (m, 1H), 5.04 (br s, 2H); LCMS method A, (ES+) 239, 241, RT=1.90 min.

### Step (ii)

### N-(2-(2-chloro-5-fluoropyrimidin-4-ylamino)phenyl)methanesulfonamide

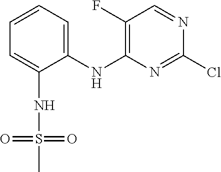

A solution of N1-(2-chloro-5-fluoropyrimidin-4-yl)benzene-1,2-diamine (1.5 g, 6.30 mmol) in pyridine (15 mL) was cooled to 0° C. before dropwise addition of methanesulfonyl chloride (0.54 mL, 6.93 mmol). The resultant solution was allowed to warm to room temperature and stirred for 18 h then diluted with water (25 mL) and ethyl acetate (25 mL). The separated organic layer was washed with 2M hydrochloric acid (2×25 mL) and brine (25 mL), dried (MgSO₄) and concentrated in vacuo to provide N-(2-(2-chloro-5-fluoropyrimidin-4-ylamino)phenyl)methanesulfonamide as a beige solid (1.45 g, 72%). ¹H NMR (d₆-DMSO) δ 9.41 (br s, 1H), 9.25 (s, 1H), 8.30 (d, 1H), 7.47-7.52 (m, 2H), 7.32 (t, 1H), 7.25 (t, 1H), 2.99 (s, 3H); LCMS method A, (ES+) 316, RT=2.26 min.

### Intermediate 1b

### N-(2-(2-chloro-5-methylpyrimidin-4-ylamino)phenyl)methanesulfonamide

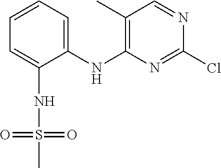

1b was made according to the procedure of 1a using 2,4-dichloro-5-methylpyrimidine instead of 2,4-dichloro-5-fluoropyrimidine in step (i). ¹H NMR (d₆-DMSO) δ 9.24 (s, 1H), 8.49 (s, 1H), 8.06 (s, 1H), 7.60, (m, 1H), 7.48 (m, 1H), 7.29 (m, 2H), 3.07 (s, 3H), 2.17 (s, 3H); LCMS method A, (ES+) 314, RT=1.73 min.

### Intermediate 1c

### N-(2-(5-bromo-2-chloropyrimidin-4-ylamino)phenyl)methanesulfonamide

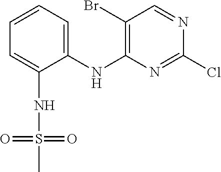

1c was made according to the procedure of 1a using 2,4-dichloro-5-bromopyrimidine instead of 2,4-dichloro-5-fluoropyrimidine in step (i). LCMS method A, (ES+) 378, RT=2.47 min.

### Intermediate 1d

### N-(2-(2-chloropyrimidin-4-ylamino)phenyl)methanesulfonamide

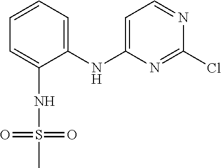

1d was made according to the procedure of 1a using 2,4-dichloropyrimidine instead of 2,4-dichloro-5-fluoropyrimidine in step (i). LCMS method A, (ES+) 297, 291, RT=1.82 min.

### Intermediate 1e

### N-(2-(2-chloro-5-fluoropyrimidin-4-ylamino)phenyl)-N-methylmethanesulfonamide

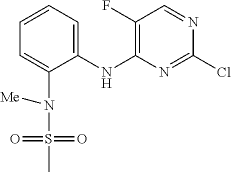

A mixture of Intermediate 1a (200 mg, 0.63 mmol), K₂CO₃ (174 mg, 1.26 mmol) and MeI (100 mg, 0.70 mmol) in DMF (5 mL) was stirred at room temperature for 18 h then diluted with water (20 mL). The mixture was extracted with ethyl acetate (25 mL), washed with water (20 mL) and brine (20 mL), dried (MgSO₄) and concentrated in vacuo. Trituration with diethyl ether afforded N-(2-(2-chloro-5-fluoropyrimidin-4-ylamino)phenyl)methane sulfonamide as a pale yellow solid (200 mg, 96%). ¹H NMR (d₆-DMSO) δ 9.43 (br s, 1H), 8.34 (d, 1H), 7.63-7.66 (m, 2H), 7.44 (t, 1H), 7.36 (t, 1H), 3.18 (s, 3H), 3.00 (t, 3H); LCMS method A, (ES+) 331, RT=2.34 min.

### Intermediate 1f

### N-(2-(2-chloro-5-nitropyrimidin-4-ylamino)phenyl)methanesulfonamide

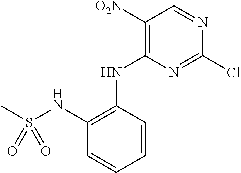

### Step (i)

### N-(2-nitrophenyl)methanesulfonamide

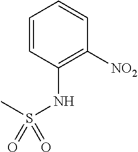

Methanesulfonyl chloride (2.7 mL, 48 mmol) was added to a solution of 2-nitroaniline (4.0 g, 29 mmol) in pyridine (10 ml), the mixture was stirred at room temperature for 18 h then poured over stirred ice. The precipitate was collected by filtration then dissolved in 2:3 THF/1M NaOH(aq) (100 mL) and stirred at room temperature for 2 h. The reaction mixture was acidified to pH 7 with 2M hydrochloric acid and extracted with ethyl acetate (3×30 mL1). The organics were washed with brine (30 mL) and water (30 mL), dried (MgSO₄) and concentrated in vacuo to afford N-(2-nitrophenyl)methanesulfonamide as an orange solid (5.0 g, 80%). ¹H NMR (d₆-DMSO) δ 9.82 (s, 1H), 8.03 (d, 1H), 7.75 (t, 1H), 7.64 (d, 1H), 7.41 (t, 1H), 3.15 (t, 3H); LCMS method A, (ES+) 217, RT=2.0 min.

### Step (ii)

### N-(2-aminophenyl)methanesulfonamide

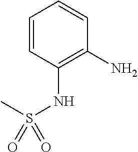

A suspension of N-(2-nitrophenyl)methanesulfonamide (5.0 g, 23 mmol) and 10% Pd/C in methanol (200 mL) was stirred under an atmosphere of hydrogen for 3 h. The mixture was filtered through Celite and concentrated in vacuo to afford N-(2-aminophenyl)methanesulfonamide as an orange solid (3.5 g, 83%). ¹H NMR (d₆-DMSO) δ 8.70 (br s, 1H), 7.05 (d, 1H), 6.98 (t, 1H), 6.73 (d, 1H), 6.55 (t, 1H), 5.14 (br s, 2H), 2.90 (s, 3H); LCMS method A, (ES+) 187, RT=0.63 min.

Step (iii)

### N-(2-(2-chloro-5-nitropyrimidin-4-ylamino)phenyl)methanesulfonamide

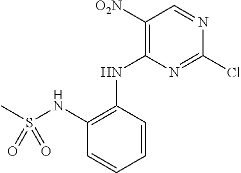

A solution of 2,4-dichloro-5-nitropyrimidine (3.65 g, 19 mmol), DIPEA (3.3 mL, 24 mmol) in THF (30 mL) was cooled to −78° C. before dropwise addition of N-(2-aminophenyl)methanesulfonamide (3.5 g, 19 mmol) in THF (70 mL). The mixture was stirred for a further 1 h then poured into water (300 mL) yielding an orange precipitate which was filtered; washed with water and cold methanol, then dried under vacuum to afford N-(2-(2-chloro-5-nitropyrimidin-4-ylamino)phenyl)methanesulfonamide (2.72 g, 40%). ¹H NMR (d₆-DMSO) δ 10.48 (s, 1H), 9.32 (s, 1H), 9.20 (s, 1H), 7.61 (d, 1H), 7.52 (d, 1H), 7.40-7.31 (m, 2H), 3.03 (s, 3H); LCMS method A, (ES+) 345, RT=2.34 min.

### Intermediate 1g

### N-(2-(2-chloro-5-fluoropyrimidin-4-ylamino)-6-methylphenyl)methanesulfonamide

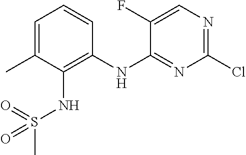

1g was made according to the procedure of 1a using 2,3-diaminotoluene instead of o-phenylenediamine in step (i). LCMS method C, (ES+) 331, 333, RT=1.72 min.

### Intermediate 1h

### N-(2-(2,5-dichloropyrimidin-4-ylamino)phenyl)methanesulfonamide

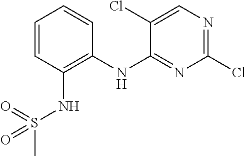

1h was made according to the procedure of 1a using 2,4,5-trichloropyrimidine instead of 2,4-dichloro-5-fluoropyrimidine in step (i). LCMS method A, (ES+) 333, 335, 337, RT=2.39 min.

### Intermediate 1i

### N-(2-(2,5-dichloropyrimidin-4-ylamino)-5-methoxyphenyl)methanesulfonamide

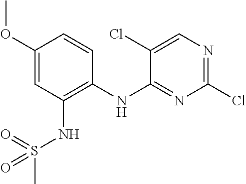

1i was made according to the procedure of 1a using 2,4,5-trichloropyrimidine and 3,4-diaminoanisole in step (i). LCMS method C, (ES+) 363, 365, RT=1.84 min.

### Intermediate 1j

### N-(2-(2-chloro-5-fluoropyrimidin-4-ylamino)phenyl)-N-ethylmethanesulfonamide

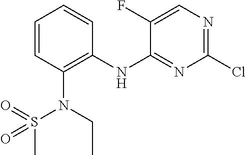

1j was made according to the procedure of 1e using ethyl iodide instead of methyl iodide. LCMS method A, (ES+) 345, 347, RT=2.46 min.

### Intermediate 2a

### N⁴-(3-aminophenyl)-5-fluoro-N²-(3,4,5-trimethoxyphenyl)pyrimidine-2,4-diamine

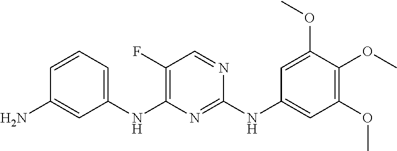

### Step (i)

### N-(3-(2-chloro-5-fluoropyrimidin-4-ylamino)phenyl)acetamide

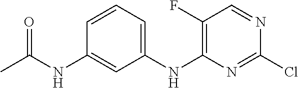

A mixture of 2,4-dichloro-5-fluoropyrimidine (3.1 g, 18.6 mmol), N-(3-aminophenyl)acetamide (3.1 g, 20.7 mmol) and DIPEA (5.6 mL, 32.2 mmol) in IPA (12 mL) were stirred at 80° C. for 16 h then concentrated in vacuo then slurried with 0.1 M hydrochloric acid (20 mL). The solid was collected at the pump, washed with water (2×20 mL), methanol (10 mL and diethyl ether (10 mL), then dried under vacuum to afford N-(3-(2-chloro-5-fluoropyrimidin-4-ylamino)phenyl)acetamide as a colourless powder (2.35 g, 94%). ¹H NMR (d₆-DMSO) δ 10.02 (d, 2H), 8.32 (s, 1H), 7.93 (s, 1H), 7.39 (d, 1H), 7.31 (m, 2H), 2.06 (s, 3H); LCMS method A, (ES+) 281, 283, RT=2.11 min.

### Step (ii)

### N-(3-(5-fluoro-2-(3,4,5-trimethoxyphenylamino)pyrimidin-4-ylamino)phenyl)acetamide

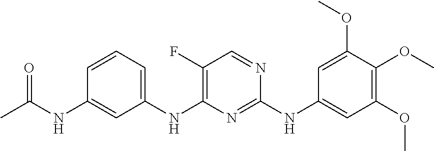

N-(3-(2-chloro-5-fluoropyrimidin-4-ylamino)phenyl)acetamide (4.70 g, 16.7 mmol), 3,4,5-trimethoxyaniline (4.63 g, 25.3 mmol) and 4M HCl in dioxane (6.5 mL, 26.0 mmol) were stirred in IPA (80 mL) at 80° C. for 20 h. The resultant precipitate was collected at the pump, washed with diethyl ether then dissolved in water (20 mL). The aqueous solution was washed with diethyl ether (10 mL), adjusted to pH 9 with sat. NaHCO₃(aq) and extracted with ethyl acetate (2×20 mL). The combined organics were washed with brine, dried (MgSO₄) and concentrated in vacuo. The crude product was purified by flash chromatography (silica gel, ethyl acetate-petrol) and recrystallisation (1:1:1 DCM/ethyl acetate/petrol) to afford N-(3-(5-fluoro-2-(3,4,5-trimethoxyphenylamino)pyrimidin-4-ylamino)phenyl)acetamide as colourless crystals (3.5 g, 49%). ¹H NMR (d₆-DMSO) δ 9.89 (s, 1H), 9.39 (s, 1H), 9.01 (s, 1H), 8.10 (d, 1H), 7.80 (d, 1H), 7.57 (d, 1H), 7.29 (d, 1H), 7.21 (t, 2H), 7.05 (s, 2H), 3.61 (s, 6H), 3.59 (s, 3H), 2.03 (s, 3H); LCMS method A, (ES+) 428, RT=1.91 min.

Step (iii)

### N⁴-(3-aminophenyl)-5-fluoro-N²-(3,4,5-trimethoxyphenyl)pyrimidine-2,4-diamine

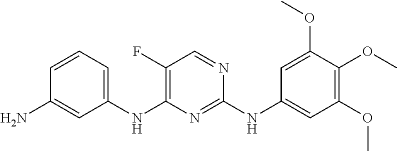

5M NaOH(aq) (8.2 mL, 41 mmol) was added to a stirred solution of N-(3-(5-fluoro-2-(3,4,5-trimethoxyphenylamino)pyrimidin-4-ylamino)phenyl)acetamide (3.5 g, 8.2 mmol) in ethanol (50 mL) at 60° C. The temperature was raised to 80° C. and stirring was continued for 2 days whereupon the mixture was concentrated by half and filtered. The residue was washed with 1:2 methanol/water, then dried in a vacuum oven overnight to afford N⁴-(3-aminophenyl)-5-fluoro-N²-(3,4,5-trimethoxyphenyl)pyrimidine-2,4-diamine as a tan solid (2.57 g, 81%). ¹H NMR (d₆-DMSO) δ 9.05 (s, 1H), 8.96 (s, 1H), 8.06 (d, 1H), 7.06 (s, 2H), 6.94 (s, 3H), 6.30 (d, 1H), 4.99 (s, 2H), 3.63 (s, 6H), 3.59 (s, 3H); LCMS method A, (ES+) 386, RT=1.75 min.

### Intermediate 2b

### N⁴-(2-aminophenyl)-5-fluoro-N²-(3,4,5-trimethoxyphenyl)pyrimidine-2,4-diamine

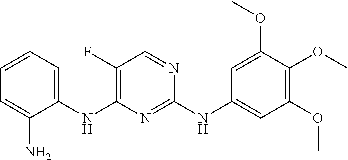

2b was made according to the procedure of 2a using (2-aminophenyl)acetamide instead of (3-aminophenyl)acetamide in step (i). ¹H NMR (d₆-DMSO) δ 8.87 (s, 1H), 8.57 (s, 1H), 8.00 (d, 1H), 7.12 (dd, 1H), 6.98-6.94 (m, 3H), 6.75 (dd, 1H), 6.56 (td, 1H), 4.92 (s, 2H), 3.54 (s, 3H), 3.47 (s, 6H); LCMS method A, (ES+) 386, RT=1.87 min.

### Intermediate 2c

### N⁴-(4-aminophenyl)-5-fluoro-N²-(3,4,5-trimethoxyphenyl)pyrimidine-2,4-diamine

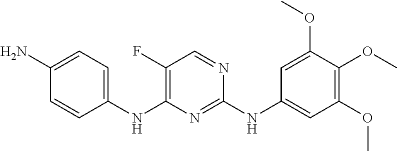

2c was made according to the procedure of 2a using (4-aminophenyl)acetamide instead of (3-aminophenyl)acetamide in step (i). ¹H NMR (d₆-DMSO) δ 9.25 (s, 1H), 9.02 (br s, 1H), 8.04 (d, 1H), 7.12 (d, 2H), 7.04 (br s, 4H), 3.62 (s, 6H), 3.59 (s, 3H); LCMS method A, (ES+) 386, RT=1.88 min.

### Intermediate 3a

### N-(2-(5-fluoro-2-(3,4,5-trimethoxyphenylamino)pyrimidin-4-ylamino)phenyl)ethenesulfonamide

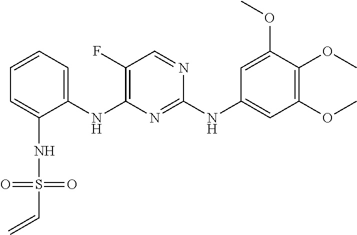

2-Chloroethanesulfonyl chloride (86 μL, 0.31 mmol) was added to a stirred solution of Intermediate 2b (100 mg, 0.26 mmol) in pyridine (2 mL) at 0° C. then warmed to room temperature. After stirring for 18 h the mixture was diluted with water (25 mL) and ethyl acetate (25 mL). The separated organic layer was washed with brine (2×25 mL), dried (MgSO₄) and concentrated in vacuo to afford N-(2-(5-fluoro-2-(3,4,5-trimethoxyphenylamino)pyrimidin-4-ylamino)phenyl)ethenesulfonamide as a brown solid.

LCMS method A, (ES+) 476, RT=2.23 min.

### Intermediate 3b

### N-(3-(5-fluoro-2-(3,4,5-trimethoxyphenylamino)pyrimidin-4-ylamino)phenyl)ethenesulfonamide

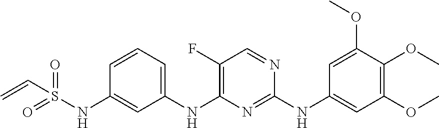

3b was made according to the procedure of Intermediate 3a using Intermediate 2a instead of Intermediate 2b. LCMS method A, (ES+) 476, RT=1.68 min.

### Example 1

### N-(2-(5-fluoro-2-(3,4,5-trimethoxyphenylamino)pyrimidin-4-ylamino)phenyl)methanesulfonamide

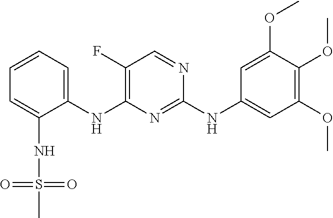

A mixture of Intermediate 1a (100 mg, 0.32 mmol), 3,4,5-trimethoxyaniline (63.6 mg, 0.35 mmol), 4M HCl in dioxane (0.1 mL) and n-butanol (2 mL) was heated in a microwave at 120° C. for 45 min. The precipitate was collected by filtration and washed with n-butanol (2×10 mL) and diethyl ether (2×10 mL) to afford N-(2-(5-fluoro-2-(3,4,5-trimethoxyphenylamino)pyrimidin-4-ylamino)phenyl)methanesulfonamide as a colourless solid (106 mg, 72%); ¹H NMR (d₆-DMSO) δ 9.21 (s, 1H), 9.05 (s, 1H), 8.67 (s, 1H), 8.13 (d, 1H), 7.79 (d, 1H), 7.41 (d, 1H), 7.26-7.22 (m, 2H), 6.96 (s, 2H), 3.56 (s, 3H), 3.52 (s, 6H), 2.92 (s, 3H); LCMS method A, (ES+) 464, RT=1.80 min.

### Example 2

### N-(2-(5-fluoro-2-(4-fluorophenylamino)pyrimidin-4-ylamino)phenyl)methanesulfonamide

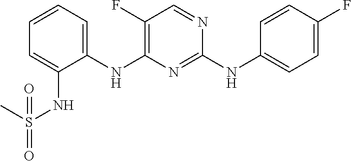

Synthesized according to the procedure in Example 1 using Intermediate 1a and 4-fluoroaniline. ¹H NMR (d₆-DMSO) δ 10.09 (br s, 1H), 9.8 (br s, 1H), 9.32 (s, 1H), 8.27 (d, 1H), 7.58 (d, 1H), 7.51 (d, 1H), 7.29-7.42 (m, 4H), 7.00 (t, 1H), 2.93 (s, 3H);

LCMS method A, (ES+) 392, RT=2.27 min.

### Example 3

### N-(2-(2-(3,5-dimethylphenylamino)-5-fluoropyrimidin-4-ylamino)phenyl)methanesulfonamide

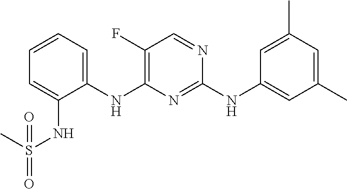

Synthesized according to the procedure in Example 1 using Intermediate 1a and 3,5-dimethylaniline. ¹H NMR (d₆-DMSO) δ 10.30 (br s, 1H), 10.20 (br s, 1H), 9.33 (s, 1H), 8.36 (d, 1H), 7.58 (d, 2H), 7.41 (t, 1H), 7.31 (t, 1H), 6.93 (s, 2H), 6.63 (s, 1H), 2.91 (s, 3H), 2.08 (s, 6H); LCMS method A, (ES+) 402, RT=2.44 min.

### Example 4

### N-(2-(2-(2,3-dihydrobenzo[b][1,4]dioxin-6-ylamino)-5-fluoropyrimidin-4-ylamino)phenyl)methanesulfonamide

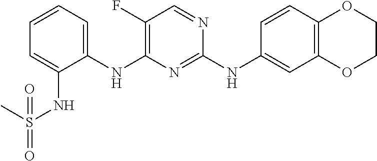

Synthesized according to the procedure in Example 1 using Intermediate 1a and 2,3-dihydrobenzo[b][1,4]dioxin-6-amine. ¹H NMR (d₆-DMSO) δ 10.05 (br s, 2H), 9.33 (s, 1H), 8.25 (d, 1H), 7.59 (d, 1H), 7.49 (d 1H), 7.36 (t, 1H), 7.26 (t, 1H), 6.91 (s, 1H), 6.81 (d, 1H), 6.67 (d, 1H), 4.19 (d, 4H), 2.94 (s, 3H); LCMS method A, (ES+) 432, RT=2.04 min.

### Example 5

### N-(2-(2-(3,5-difluorophenylamino)-5-fluoropyrimidin-4-ylamino)phenyl)methanesulfonamide

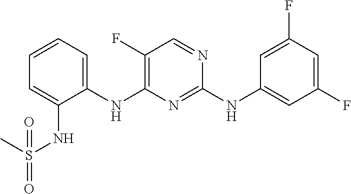

Synthesized according to the procedure in Example 1 using Intermediate 1a and 3,5-difluoroaniline. ¹H NMR (d₆-DMSO) δ 9.83 (br s, 1H), 9.28 (s, 1H), 9.15 (br s, 1H), 8.22 (d, 1H), 7.67 (d, 1H), 7.42 (d 1H), 7.25-7.35 (m, 4H), 6.64 (t, 1H), 2.93 (s, 3H); LCMS method A, (ES+) 410, RT=2.14 min.

### Example 6

### N-(2-(5-fluoro-2-(3-fluorophenylamino)pyrimidin-4-ylamino)phenyl)methanesulfonamide

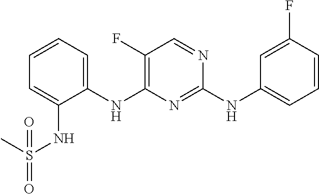

Synthesized according to the procedure in Example 1 using Intermediate 1a and 3-fluoroaniline. ¹H NMR (d₆-DMSO) δ 10.02 (br s, 1H), 9.55 (br s, 1H) 9.31 (s, 1H), 8.28 (d, 1H), 7.67 (d, 1H), 7.51 (d 1H), 7.41 (d, 1H), 7.17-7.35 (m, 2H), 7.18 (s, 2H), 6.70-6.74 (m, 1H), 2.93 (s, 3H); LCMS method A, (ES+) 392, RT=2.10 min.

### Example 7

### N-(2-(2-(4-(dimethylamino)phenylamino)-5-fluoropyrimidin-4-ylamino)phenyl)methanesulfonamide

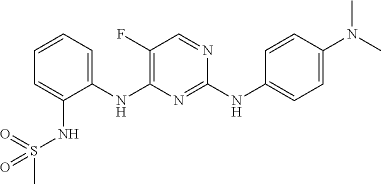

Synthesized according to the procedure in Example 1 using Intermediate 1a and N1,N1-dimethylbenzene-1,4-diamine. ¹H NMR (d₆-DMSO) δ 9.87 (br s, 2H), 9.32 (s, 1H), 8.24 (d, 1H), 7.67 (d, 1H), 7.49-7.55 (m, 4H), 7.33-7.1 (m, 3H), 3.05, (br s, 6H), 2.93 (s, 3H); LCMS method A, (ES+) 417, RT=1.97 min.

### Example 8

### N-(2-(2-(3,5-bis(trifluoromethyl)phenylamino)-5-fluoropyrimidin-4-ylamino)phenyl)methanesulfonamide

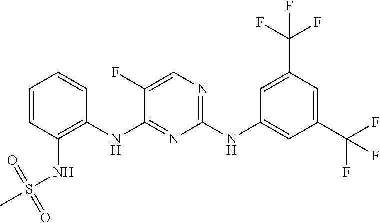

Synthesized according to the procedure in Example 1 using Intermediate 1a and 3,5-bis(trifluoromethyl)aniline. ¹H NMR (d₆-DMSO) δ 10.00 (br s, 1H), 9.26 (s, 1H), 9.5 (br s, 1H), 8.28 (d, 3H), 7.70 (d, 1H), 7.49 (d, 2H), 7.28 (d, 2H), 2.93 (s, 3H); LCMS method A, (ES+) 510, RT=3.2 min.

### Example 9

### N-(2-(5-fluoro-2-(4-(trifluoromethyl)phenylamino)pyrimidin-4-ylamino)phenyl)methanesulfonamide

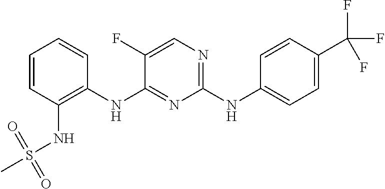

Synthesized according to the procedure in Example 1 using Intermediate 1a and 4-trifluormethylaniline. ¹H NMR (d₆-DMSO) δ 10.23 (br s, 1H), 9.62 (br s, 1H), 9.34 (br s, 1H), 8.30 (d, 1H), 7.65 (d, 3H), 7.52 (d, 1H), 7.45 (d, 2H), 7.35 (d, 2H), 2.93 (s, 3H). LCMS method A, (ES+) 442, RT=2.8 min.

### Example 10

### N-(2-(2-(4-chloro-3-methoxyphenylamino)-5-fluoropyrimidin-4-ylamino)phenyl)methanesulfonamide

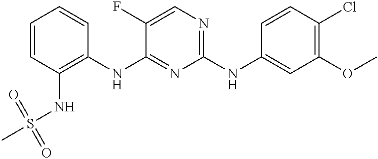

Synthesized according to the procedure in Example 1 using Intermediate 1a and 4-chloro-3-methoxyaniline. ¹H NMR (d₆-DMSO) δ 9.45 (br s, 1H), 9.24 (br s, 1H), 8.95 (br s, 1H), 8.15 (d, 1H), 7.72 (d, 1H), 7.46 (d, 1H), 7.40 (d, 2H), 7.29 (d, 2H), 7.21, (1H), 7.13 (d, 1H), 2.93 (s, 3H). LCMS method A, (ES+) 438, RT=2.6 min.

### Example 11

### N-(2-(5-fluoro-2-(4-methyl-3-nitrophenylamino)pyrimidin-4-ylamino)phenyl)methanesulfonamide

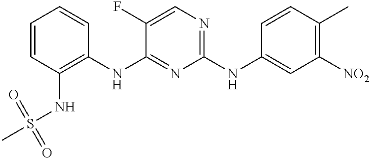

Synthesized according to the procedure in Example 1 using Intermediate 1a and 4-methyl-3-nitroaniline. ¹H NMR (d₆-DMSO) δ 10.11 (br s, 1H), 9.51 (br s, 1H), 9.30 (s, 1H), 8.27 (d, 1H), 8.13 (d, 1H), 7.63 (m, 2H), 7.47 (d, 2H), 7.24-7.30 (m, 3H), 2.86 (s, 3H), 2.39 (s, 3H); LCMS method A, (ES+) 433, RT=2.6 min.

### Example 12

### N-(2-(2-(3-chlorophenylamino)-5-fluoropyrimidin-4-ylamino)phenyl)methanesulfonamide

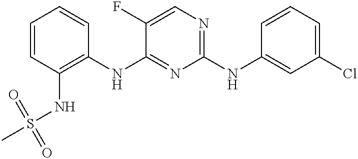

Synthesized according to the procedure in Example 1 using Intermediate 1a and 3-chloroaniline. ¹H NMR (d₆-DMSO) δ 10.08 (br s, 1H), 9.62 (br s, 1H), 9.31 (s, 1H), 8.29 (d, 1H), 7.65 (d, 2H), 7.49 (d, 1H), 7.31-7.34 (m, 3H), 7.16 (t, 1H), 6.95 (d, 1H), 2.93 (s, 3H); LCMS method A, (ES+) 408, RT=2.6 min.

### Example 13

### N-(2-(2-(3-ethoxyphenylamino)-5-fluoropyrimidin-4-ylamino)phenyl)methanesulfonamide

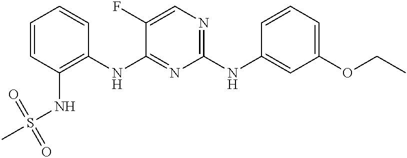

Synthesized according to the procedure in Example 1 using Intermediate 1a and 3-ethoxyaniline. ¹H NMR (d₆-DMSO) δ 9.52 (br s, 1H), 9.26 (s, 1H), 9.21 (br s, 1H), 8.29 (d, 1H), 7.73 (t, 1H), 7.47 (t, 1H), 7.30 (dd, 3H), 7.16 (br s, 1H), 7.02-7.04 (m, 2H), 6.47 (d, 1H), 3.80 (q, 2H), 2.93 (s, 3H), 1.28 (t, 3H); LCMS method A, (ES+) 418, RT=2.4 min.

### Example 14

### N-(2-(2-(3-acetylphenylamino)-5-fluoropyrimidin-4-ylamino)phenyl)methanesulfonamide

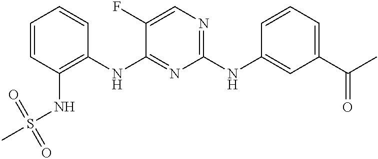

Synthesized according to the procedure in Example 1 using Intermediate 1a and 3′-aminoacetophenone. ¹H NMR (d₆-DMSO) δ 9.94 (br s, 1H), 9.29 (s, 1H), 9.24 (br s, 1H), 8.26 (d, 1H), 7.72-7.74 (m, 4H), 7.64 (d, 2H), 7.36 (t, 2H), 2.93 (s, 3H), 2.48 (s, 3H); LCMS method A, (ES+) 418, RT=2.4 min.

### Example 15

### N-(2-(5-fluoro-2-(3-(methylthio)phenylamino)pyrimidin-4-ylamino)phenyl)methanesulfonamide

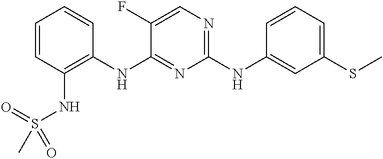

Synthesized according to the procedure in Example 1 using Intermediate 1a and 3-(methylthio)aniline. ¹H NMR (d₆-DMSO) δ 9.74 (br s, 1H), 9.42 (br s, 1H), 9.32 (s, 1H), 8.28 (d, 1H), 7.76 (d, 1H), 7.54 (t, 1H), 7.44 (s, 1H), 7.36 (t, 2H), 7.34 (d, 1H), 7.15 (t, 1H), 6.88 (d, 1H), 2.98 (s, 3H), 2.40 (s, 3H); LCMS method A, (ES+) 420, RT=2.4 min.

### Example 16

### N-(2-(2-(benzo[d]thiazol-5-ylamino)-5-fluoropyrimidin-4-ylamino)phenyl)methanesulfonamide

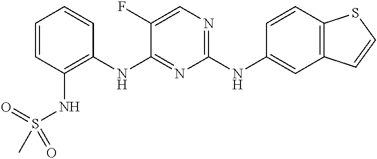

Synthesized according to the procedure in Example 1 using Intermediate 1a and 6-aminobenzothiazole. ¹H NMR (d₆-DMSO) δ 9.86 (br s, 1H), 9.34 (s, 1H), 9.30 (s, 1H), 8.35 (br s, 1H), 8.26 (d, 1H), 7.94 (d, 1H), 7.79 (d, 1H), 7.61 (s, 1H), 7.47 (d, 1H), 7.30-7.33 (m, 2H), 2.95 (s, 3H); LCMS method A, (ES+) 431, RT=2.4 min.

### Example 17

### N-(2-(2-(3-(1H-pyrazol-1-yl)phenylamino)-5-fluoropyrimidin-4-ylamino)phenyl)methanesulfonamide

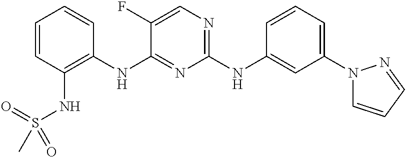

Synthesized according to the procedure in Example 1 using Intermediate 1a and 3-pyrazolylphenylamine. ¹H NMR (d₆-DMSO) δ 9.96 (br s, 1H), 9.50 (s, 1H), 9.31 (s, 1H), 8.28 (d, 1H), 8.21 (d, 1H), 7.96 (s, 1H), 7.73 (s, 1H), 7.69 (d, 1H), 7.40 (d, 2H), 7.26 (t, 2H), 7.12 (m, 2H), 6.53 (t, 1H), 2.95 (s, 3H); LCMS method A, (ES+) 440, RT=2.4 min.

### Example 18

### N-(2-(5-fluoro-2-(2-methylbenzo[d]thiazol-5-ylamino)pyrimidin-4-ylamino)phenyl)methanesulfonamide

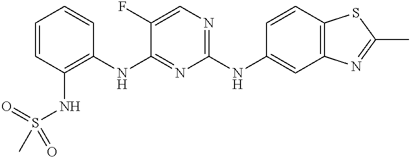

Synthesized according to the procedure in Example 1 using Intermediate 1a and 5-amino-2-methylbenzothiazole. ¹H NMR (d₆-DMSO) δ 10.23 (br s, 1H), 9.80 (br s, 1H), 9.33 (s, 1H), 8.30 (d, 1H), 8.12 (s, 1H), 7.72 (d, 1H), 7.61 (d, 1H), 7.57 (d, 1H), 7.44 (t, 2H), 7.32-7.36 (m, 2H), 2.92 (s, 3H), 2.74 (s, 3H); LCMS method A, (ES+) 445, RT=2.4 min.

### Example 19

### N-(2-(2-(3-chloro-4-methoxyphenylamino)-5-fluoropyrimidin-4-ylamino)phenyl)methanesulfonamide

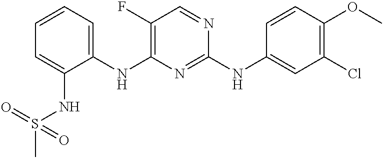

Synthesized according to the procedure in Example 1 using Intermediate 1a and 3-chloro-4-methoxyaniline. ¹H NMR (d₆-DMSO) δ 10.08 (br s, 1H), 9.62 (br s, 1H), 9.31 (s, 1H), 8.27 (d, 1H), 7.62 (d, 1H), 7.57 (d, 1H), 7.46-7.51 (m, 1H), 7.30-7.34 (m, 2H), 7.27 (d, 1H), 6.98 (d, 1H), 3.79 (s, 3H), 2.93 (s, 3H); LCMS method A, (ES+) 437, RT=2.6 min.

### Example 20

### N-(2-(5-fluoro-2-(4-methyl-3-oxo-3,4-dihydro-2H-benzo[b][1,4]oxazin-6-ylamino)pyrimidin-4-ylamino)phenyl)methanesulfonamide

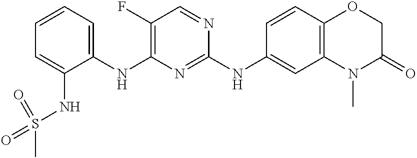

Synthesized according to the procedure in Example 1 using Intermediate 1a and 6-amino-4-methyl-2H-1,4-benzoxazin-3(4H)-one. ¹H NMR (d₆-DMSO) δ 10.25 (br s, 1H), 10.16 (br s, 1H), 9.36 (s, 1H), 8.30 (d, 1H), 7.55 (d, 1H), 7.50 (d, 1H), 7.46 (d, 1H), 7.32 (t, 1H), 7.27 (t, 1H), 7.15 (s, 1H), 7.01 (d, 3H), 6.82 (d, 1H), 4.59 (s, 2H), 2.99 (s, 3H), 2.94 (s, 3H); LCMS method A, (ES+) 459, RT=2.8 min.

### Example 21

### N-(2-(2-(3-(1H-1,2,4-triazol-1-yl)phenylamino)-5-fluoropyrimidin-4-ylamino)phenyl)methanesulfonamide

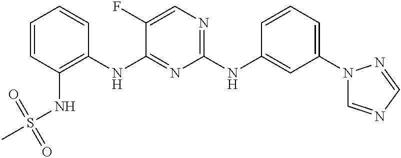

Synthesized according to the procedure in Example 1 using Intermediate 1a and 3-(1H-1,2,4-triazol-1-yl)aniline. ¹H NMR (d₆-DMSO) δ 9.47 (br s, 1H), 9.25 (br s, 1H), 9.14 (s, 1H), 8.77 (br s, 1H), 8.18 (d, 2H), 7.82 (d, 1H), 7.79 (d, 2H), 7.59 (d, 2H), 7.49 (d, 1H), 7.30-7.36 (m, 2H), 2.93 (s, 3H); LCMS method A, (ES+) 441, RT=2.1 min.

### Example 22

### N-(2-(2-(3-(3,5-dimethyl-1H-pyrazol-1-yl)phenylamino)-5-fluoropyrimidin-4-ylamino)phenyl)methanesulfonamide

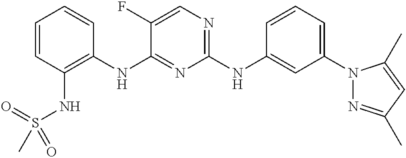

Synthesized according to the procedure in Example 1 using Intermediate 1a and 3-(3,5-dimethyl-1H-pyrazol-1-yl)aniline. ¹H NMR (d₆-DMSO) δ 10.45 (br s, 1H), 10.09 (br s, 1H), 9.37 (s, 1H), 8.35 (br s, 1H), 7.60 (d, 2H), 7.55 (d, 2H), 7.48 (d, 2H), 7.34-7.37 (m, 2H), 7.24 (d, 2H), 6.06 (s, 2H), 2.92 (s, 3H); 2.22 (s, 3H), 2.17 (s, 3H); LCMS method A, (ES+) 468, RT=2.4 min.

### Example 23

### N-(2-(5-fluoro-2-(3-oxo-3,4-dihydro-2H-benzo[b][1,4]oxazin-7-ylamino)pyrimidin-4-ylamino)phenyl)methanesulfonamide

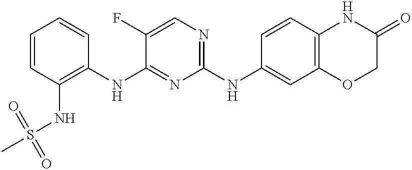

Synthesized according to the procedure in Example 1 using Intermediate 1a and 7-amino-2H-1,4-benzoxazin-3(4H)-one. ¹H NMR (d₆-DMSO) δ 10.49 (br s, 1H), 9.14 (br s, 1H), 8.71 (br s, 1H), 8.11 (d, 1H), 7.90 (d, 1H), 7.42 (d, 1H), 7.32 (s, 1H), 7.20-7.24 (m, 2H), 7.13 (d, 1H), 6.66 (d, 1H), 4.50 (s, 2H), 2.90 (s, 3H); LCMS method A, (ES+) 445, RT=2.2 min.

### Example 24

### N-(2-(5-fluoro-2-(3-(5-methyl-4H-1,2,4-triazol-3-yl)phenylamino)pyrimidin-4-ylamino)phenyl)methanesulfonamide

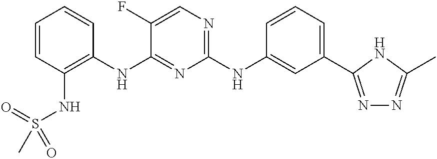

Synthesized according to the procedure in Example 1 using Intermediate 1a and 3-(5-methyl-4H-1,2,4-triazol-3-yl)aniline. ¹H NMR (d₆-DMSO) δ 13.47 (br s, 1H), 9.38 (br s, 1H), 9.25 (br s, 1H), 8.75 (s, 1H), 8.17 (d, 1H), 7.80 (d, 1H), 7.71 (d, 2H), 7.63 (s, 1H), 7.31-7.34 (m, 2H), 2.92 (s, 3H), 2.38 (s, 3H); LCMS method A, (ES+) 455, RT=1.9 min.

### Example 25

### N-(2-(5-fluoro-2-(4-methyl-3-oxo-3,4-dihydro-2H-benzo[b][1,4]oxazin-7-ylamino)pyrimidin-4-ylamino)phenyl)methanesulfonamide

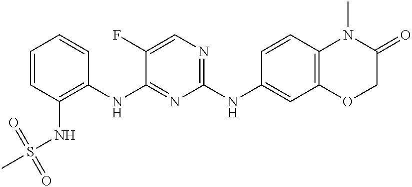

Synthesized according to the procedure in Example 1 using Intermediate 1a and 7-amino-4-methyl-2H-1,4-benzoxazin-3(4H)-one. ¹H NMR (d₆-DMSO) δ 9.25 (br s, 1H), 9.22 (s, 1H), 8.68 (s, 1H), 8.14 (d, 1H), 7.86 (d, 1H), 7.45 (d, 1H), 7.40 (d, 1H), 7.32 (t, 1H), 7.25 (t, 2H), 6.95 (d, 1H), 4.58 (s, 2H), 3.23 (s, 3H), 2.93 (s, 3H); LCMS method A, (ES+) 459, RT=2.1 min.

### Example 26

### N-(2-(2-(3-(difluoromethoxy)phenylamino)-5-fluoropyrimidin-4-ylamino)phenyl)methanesulfonamide

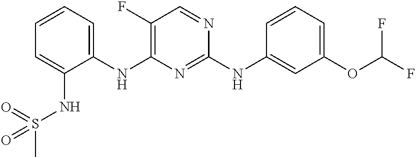

Synthesized according to the procedure in Example 1 using Intermediate 1a and 3-(difluoromethoxy)aniline. ¹H NMR (d₆-DMSO) δ 10.11 (br s, 1H), 9.69 (s, 1H), 9.32 (s, 1H), 8.30 (d, 1H), 7.65 (d, 1H), 7.50 (d, 1H), 7.30-7.35 (m, 4H), 7.18 (t, 1H), 7.09 (t, 1H), 6.75 (d, 1H), 2.93 (s, 3H); LCMS method A, (ES+) 439, RT=2.35 min.

### Example 27

### N-(2-(2-(4-(difluoromethoxy)phenylamino)-5-fluoropyrimidin-4-ylamino)phenyl)methanesulfonamide

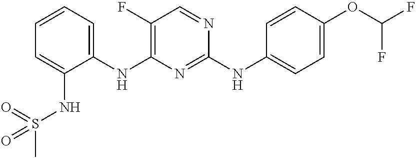

Synthesized according to the procedure in Example 1 using Intermediate 1a and 4-(difluoromethoxy)aniline. ¹H NMR (d₆-DMSO) δ 10.28 (br s, 1H), 10.02 (s, 1H), 9.35 (s, 1H), 8.31 (d, 1H), 7.58 (d, 1H), 7.53 (d, 1H), 7.31-7.41 (m, 4H), 7.13 (t, 1H), 6.95 (d, 1H), 2.93 (s, 3H). LCMS method A, (ES+) 439, RT=2.35 min.

### Example 28

### N-(2-(5-fluoro-2-(4-(trifluoromethoxy)phenylamino)pyrimidin-4-ylamino)phenyl)methanesulfonamide

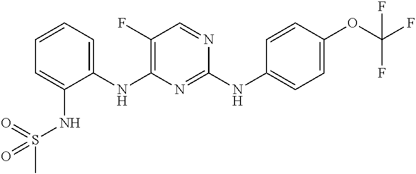

Synthesized according to the procedure in Example 1 using Intermediate 1a and 4-trifluoromethoxyaniline. ¹H NMR (d₆-DMSO) δ 10.16 (br s, 1H), 9.78 (s, 1H), 9.34 (s, 1H), 8.29 (d, 1H), 7.61 (d, 1H), 7.51 (d, 3H), 7.29-7.37 (m, 2H), 7.13 (d, 2H), 2.93 (s, 3H); LCMS method A, (ES+) 458, RT=2.35 min.

### Example 29

### N-(2-(5-fluoro-2-(3-(trifluoromethoxy)phenylamino)pyrimidin-4-ylamino)phenyl)methanesulfonamide

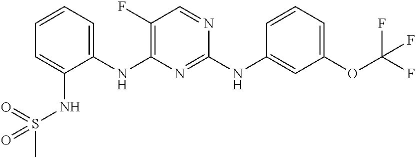

Synthesized according to the procedure in Example 1 using Intermediate 1a and 3-trifluoromethoxyaniline. ¹H NMR (d₆-DMSO) δ 10.10 (br s, 1H), 9.56 (s, 1H), 9.30 (s, 1H), 8.29 (d, 1H), 7.65 (d, 1H), 7.58 (s, 3H), 7.58 (d, 1H), 7.40 (d, 1H). 7.25-7.32 (m, 3H), 6.68 (d, 1H), 2.92 (s, 3H); LCMS method A, (ES+) 458, RT=2.41 min.

### Example 30

### N-(2-(2-(4-chlorophenylamino)-5-fluoropyrimidin-4-ylamino)phenyl)methanesulfonamide

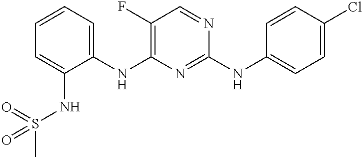

Synthesized according to the procedure in Example 1 using Intermediate 1a and 4-chloroaniline. ¹H NMR (d₆-DMSO) δ 10.07 (br s, 1H), 9.78 (s, 1H), 9.31 (s, 1H), 8.28 (d, 1H), 7.62 (d, 1H), 7.52 (d, 1H), 7.45 (d, 2H), 7.33-7.37 (m, 2H), 7.17 (d, 2H), 2.93 (s, 3H); LCMS method A, (ES+) 408, RT=2.3 min.

### Example 31

### N-(2-(5-fluoro-2-(3-(1,1,2,2-tetrafluoroethoxy)phenylamino)pyrimidin-4-ylamino)phenyl)methanesulfonamide

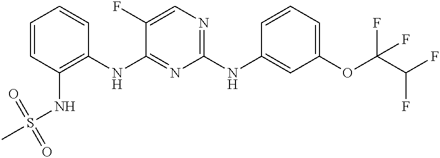

Synthesized according to the procedure in Example 1 using Intermediate 1a and 3-(1,1,2,2-tetrafluoroethoxy)aniline. ¹H NMR (d₆-DMSO) δ 10.25 (br s, 1H), 10.12 (s, 1H), 9.35 (s, 1H), 8.29 (d, 1H), 7.70 (d, 1H), 7.65 (s, 3H), 7.60 (d, 1H), 7.45 (d, 1H). 7.20-7.35 (m, 3H), 7.12 (d, 1H), 2.94 (s, 3H); LCMS method A, (ES+) 490, RT=2.40 min.

### Example 32

### N-(2-(2-(1H-indazol-6-ylamino)-5-fluoropyrimidin-4-ylamino)phenyl)methanesulfonamide

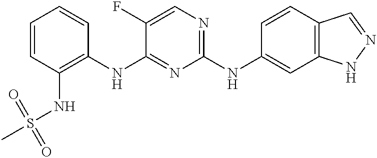

Synthesized according to the procedure in Example 1 using Intermediate 1a and 1H-indazol-6-amine. ¹H NMR (d₆-DMSO) δ 10.23 (br s, 1H), 9.85 (s, 1H), 9.35 (s, 1H), 8.31 (d, 1H), 7.79 (d, 1H), 7.70 (t, 1H), 7.66 (s, 1H), 7.57 (d, 1H), 7.51 (t, 1H), 7.34 (t, 2H). 7.16 (d, 1H), 2.95 (s, 3H); LCMS method A, (ES+) 414, RT=2.01 min.

### Example 33

### N-(2-(2-(1H-benzo[d][1,2,3]-triazol-6-ylamino)-5-fluoropyrimidin-4-ylamino)phenyl)methanesulfonamide

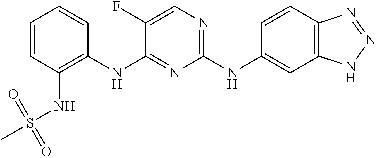

Synthesized according to the procedure in Example 1 using Intermediate 1a and 1H-benzo[d][1,2,3]triazol-6-amine. ¹H NMR (d₆-DMSO) δ 10.22 (br s, 1H), 9.76 (s, 1H), 9.35 (s, 1H), 8.32 (d, 1H), 8.02 (br s, 1H), 7.77 (d, 1H), 7.72 (t, 1H), 7.51 (t, 1H), 7.42 (d, 1H), 7.34 (t, 2H). 2.95 (s, 3H); LCMS method A, (ES+) 415, RT=1.98 min.

### Example 34

### N-(2-(5-fluoro-2-(phenylamino)pyrimidin-4-ylamino)phenyl)methanesulfonamide

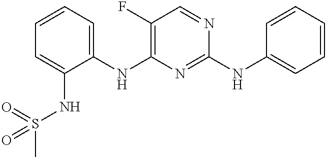

Synthesized according to the procedure in Example 1 using Intermediate 1a and aniline. ¹H NMR (d₆-DMSO) δ 9.29 (s, 1H), 8.22 (d, 1H), 7.66 (d, 1H), 7.49 (d, 1H), 7.42 (d, 2H), 7.35-7.28 (m, 2H), 7.14 (t, 2H), 6.94 (t, 1H), 2.92 (s, 3H); LCMS method B, (ES+) 374, RT=5.17 min.

### Example 35

### N-(2-(5-fluoro-2-(4-methoxyphenylamino)pyrimidin-4-ylamino)phenyl)methanesulfonamide

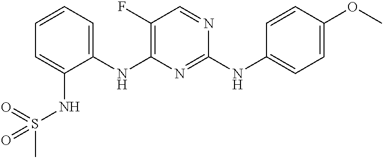

Synthesized according to the procedure in Example 1 using Intermediate 1a and 4-methoxyaniline. ¹H NMR (d₆-DMSO) δ 10.02 (br s, 2H), 9.32 (s, 1H), 8.23 (d, 1H), 7.57 (d, 1H), 7.50 (d, 1H), 7.38-7.21 (m, 4H), 6.75 (d, 2H), 3.56 (s, 3H), 2.93 (s, 3H); LCMS method B, (ES+) 404, RT=4.59 min.

### Example 36

### N-(2-(2-(3,4-dimethoxyphenylamino)-5-fluoropyrimidin-4-ylamino)phenyl)methanesulfonamide

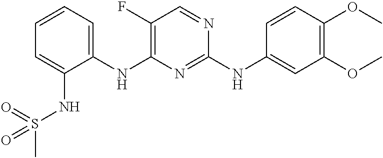

Synthesized according to the procedure in Example 1 using Intermediate 1a and 3,4-dimethoxyaniline. ¹H NMR (d₆-DMSO) δ 10.14-10.04 (br s, 2H), 9.35 (s, 1H), 8.26 (d, 1H), 7.55 (d, 1H), 7.50 (d, 1H), 7.36-7.24 (m, 2H), 6.98 (d, 1H), 6.80 (dd, 2H), 3.70 (s, 3H), 3.49 (s, 3H), 2.93 (s, 3H); LCMS method B, (ES+) 434, RT=4.29 min.

### Example 37

### N-(2-(2-(3,5-dimethoxyphenylamino)-5-fluoropyrimidin-4-ylamino)phenyl)methanesulfonamide

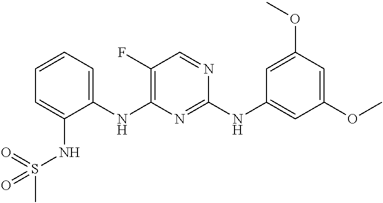

Synthesized according to the procedure in Example 1 using Intermediate 1a and 3,5-dimethoxyaniline. ¹H NMR (d₆-DMSO) δ 9.22 (s, 1H), 9.13 (s, 1H), 8.67 (s, 1H), 8.15 (d, 1H), 7.83 (d, 1H), 7.41 (d, 1H), 7.30-7.20 (m, 2H), 6.88 (d, 2H), 6.02 (t, 1H), 3.57 (s, 6H), 2.93 (s, 3H); LCMS method B, (ES+) 434, RT=5.84 min.

### Example 38

### N-(2-(5-fluoro-2-(3-methoxyphenylamino)pyrimidin-4-ylamino)phenyl)methanesulfonamide

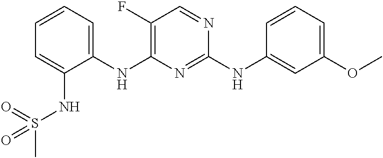

Synthesized according to the procedure in Example 1 using Intermediate 1a and 3-methoxyaniline. ¹H NMR (d₆-DMSO) δ 9.24 (s, 1H), 9.18 (s, 1H), 8.67 (s, 1H), 8.15 (d, 1H), 7.84 (dd, 1H), 7.43 (dd, 1H), 7.33-7.23 (m, 4H), 7.17 (d, 1H), 6.43 (dd, 1H), 3.60 (s, 3H), 2.93 (s, 3H); LCMS method B, (ES+) 404, RT=5.52 min.

### Example 39

### N-(4-(5-fluoro-2-(3,4,5-trimethoxyphenylamino)pyrimidin-4-ylamino)phenyl)thiophene-3-sulfonamide

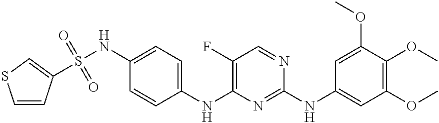

Synthesized according to the procedure of Intermediate 3a using Intermediate 2c and thiophene-3-sulfonyl chloride. ¹H NMR (d₆-DMSO) δ 10.28 (br s, 1H), 9.31 (s, 1H), 9.05 (br s, 1H), 9.04 (s, 1H), 8.08-8.11 (m, 2H), 7.69-7.74 (m, 3H), 7.24-7.25 (m, 1H), 7.02-7.05 (m, 4H), 3.65 (s, 6H), 3.60 (s, 3H); LCMS method A, (ES+) 532, RT=2.20 min.

### Example 40

### N-(4-(5-fluoro-2-(3,4,5-trimethoxyphenylamino)pyrimidin-4-ylamino)phenyl)thiophene-2-sulfonamide

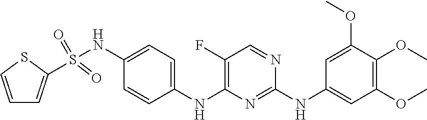

Synthesized according to the procedure of Intermediate 3a using Intermediate 2c and thiophene-2-sulfonyl chloride. ¹H NMR (d₆-DMSO) δ10.12 (br s, 1H), 9.28 (s, 1H), 9.04 (s, 1H), 8.08-8.11 (m, 1H), 7.87-7.89 (m, 1H), 7.75-7.77 (m, 2H), 7.49-7.50 (m, 1H), 7.02-7.05 (m, 4H), 3.64 (s, 6H), 3.61 (s, 3H); LCMS method A, (ES+) 532, RT=2.24 min.

### Example 41

### N-(4-(5-fluoro-2-(3,4,5-trimethoxyphenylamino)pyrimidin-4-ylamino)phenyl)pyridine-3-sulfonamide

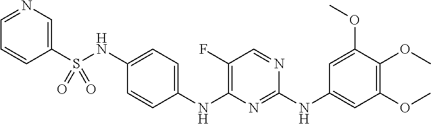

Synthesized according to the procedure of Intermediate 3a using Intermediate 2c and pyridine-3-sulfonyl chloride. ¹H NMR (d₆-DMSO) δ 10.12 (br s, 1H), 9.28 (s, 1H), 9.04 (s, 1H), 8.08-8.11 (m, 1H), 7.87-7.89 (m, 1H), 7.75-7.77 (m, 2H), 7.49-7.50 (m, 1H), 7.02-7.05 (m, 4H), 3.64 (s, 6H), 3.61 (s, 3H); LCMS method A, (ES+) 527, RT=2.10 min.

### Example 42

### N-(4-(5-fluoro-2-(3,4,5-trimethoxyphenylamino)pyrimidin-4-ylamino)phenyl)-1-methyl-1H-imidazole-4-sulfonamide

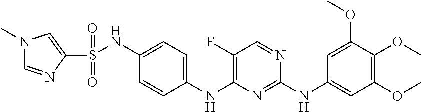

Synthesized according to the procedure of Intermediate 3a using Intermediate 2c and 1-methyl-1H-imidazole-4-sulfonyl chloride. ¹H NMR (d₆-DMSO) δ 10.14 (s, 1H), 9.25 (s, 1H), 9.02 (s, 1H), 8.57-8.59 (m, 1H), 8.07 (d, 1H), 7.76 (d, 2H), 7.67 (d, 1H), 7.39 (t, 1H), 7.08 (d, 2H), 7.02 (s, 2H), 3.65 (s, 3H), 3.61 (br s, H). LCMS method A, (ES+) 530, RT=2.03 min.

### Example 43

### N-(4-(5-fluoro-2-(3,4,5-trimethoxyphenylamino)pyrimidin-4-ylamino)phenyl)-1-phenylmethanesulfonamide

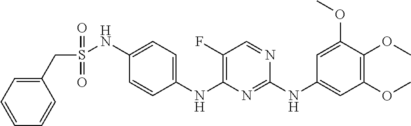

Synthesized according to the procedure of Intermediate 3a using Intermediate 2c and phenylmethanesulfonyl chloride. ¹H NMR (d₆-DMSO) δ 10.10 (s, 1H), 9.25 (s, 1H), 9.02 (s, 1H), 8.57-8.59 (m, 1H), 8.07 (d, 1H), 7.76 (d, 2H), 7.67 (d, 1H), 7.39 (t, 1H), 7.09 (d, 2H), 7.02 (s, 2H), 3.65 (s, 2H), 3.61 (br s, 9H); LCMS method A, (ES+) 540, RT=2.8 min.

### Example 44

### N-(4-(5-fluoro-2-(3,4,5-trimethoxyphenylamino)pyrimidin-4-ylamino)phenyl)benzenesulfonamide

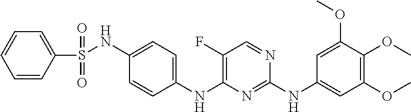

Synthesized according to the procedure of Intermediate 3a using Intermediate 2c and benzenesulfonyl chloride. ¹H NMR (d₆-DMSO) δ 10.17 (br s, 1H), 9.27 (br s, 1H), 9.03 (br s, 1H), 8.07 (d, 2H), 7.70-7.75 (m, 4H), 7.53-7.61 (m, 3H), 7.01 (t, 4H), 3.65 (s, 6H), 3.61 (s, 3H); LCMS method A, (ES+) 526, RT=2.6 min.

### Example 45

### 2,2,2-trifluoro-N-(4-(5-fluoro-2-(3,4,5-trimethoxyphenylamino)pyrimidin-4-ylamino)phenyl)ethanesulfonamide

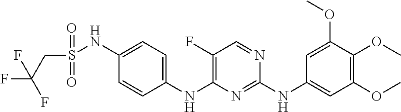

Synthesized according to the procedure of Intermediate 3a using Intermediate 2c and 2,2,2-trifluoroethanesulfonyl chloride. ¹H NMR (d₆-DMSO) δ 10.12 (br s, 1H), 9.94 (s, 1H), 9.85 (br s, 1H), 8.21 (d, 1H), 7.85 (d, 2H), 7.15 (d, 2H), 6.66 (d, 2H), 3.81 (s, 3H), 3.76 (q, 2H), 3.67 (s, 6H); LCMS method A, (ES+) 532, RT=2.42 min.

### Example 46

### 2,2,2-trifluoro-N-(2-(5-fluoro-2-(3,4,5-trimethoxyphenylamino)pyrimidin-4-ylamino)phenyl)ethanesulfonamide

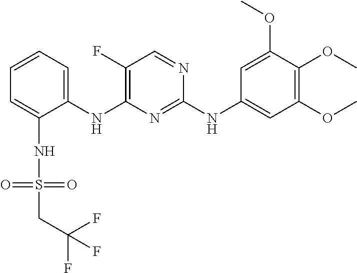

Synthesized according to the procedure of Intermediate 3a using Intermediate 2b and 2,2,2-trifluoroethanesulfonyl chloride. ¹H NMR (CDCl₃) δ 7.95 (s, 1H), 7.58 (d, 1H), 7.49 (d, 1H), 7.32 (m, 2H), 6.64 (s, 2H), 3.81 (s, 3H), 3.76 (q, 2H), 3.67 (s, 6H); LCMS method A, (ES+) 532, RT=2.46 min.

### Example 47

### N-(2-(5-fluoro-2-(3,4,5-trimethoxyphenylamino)pyrimidin-4-ylamino)phenyl)benzenesulfonamide

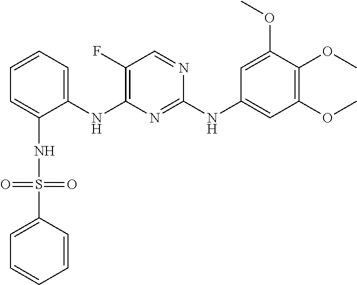

Synthesized according to the procedure of Intermediate 3a using Intermediate 2b and benzenesulfonyl chloride. ¹H NMR (d₆-DMSO) δ 9.79 (s, 1H), 8.96 (s, 1H), 8.39 (s, 1H), 8.04 (d, 1H), 7.65 (d, 1H), 7.56 (dd, 2H), 7.31 (m, 3H), 7.15 (m, 3H), 6.87 (s, 2H), 3.57 (s, 3H), 3.48 (s, 6H); LCMS method A, (ES+) 526, RT=2.47 min.

### Example 48

### N-(2-(5-fluoro-2-(3,4,5-trimethoxyphenylamino)pyrimidin-4-ylamino)phenyl)-1-phenylmethanesulfonamide

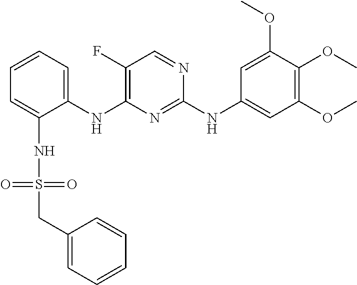

Synthesized according to the procedure of Intermediate 3a using Intermediate 2b and phenylmethanesulfonyl chloride. ¹H NMR (d₆-DMSO) δ 9.28 (s, 1H), 9.05 (s, 1H), 8.64 (s, 1H), 8.13 (d, 1H), 7.71 (dd, 1H), 7.49 (dd, 1H), 7.30 (m, 2H), 7.26 (m, 6H), 6.94 (s, 1H), 4.34 (s, 2H), 3.54 (s, 3H), 3.48 (s, 6H); LCMS method A, (ES+) 540, RT=2.52 min.

### Example 49

### N-(2-(5-fluoro-2-(3,4,5-trimethoxyphenylamino)pyrimidin-4-ylamino)phenyl)thiopehene-3-sulfonamide

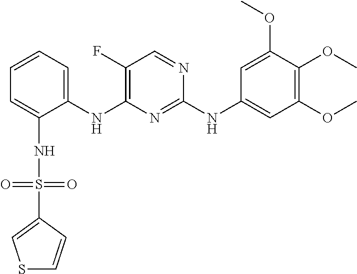

Synthesized according to the procedure of Intermediate 3a using Intermediate 2b and thiophene-3-sulfonyl chloride. ¹H NMR (d₆-DMSO) δ 9.78 (s, 1H), 9.00 (s, 1H), 8.46 (s, 1H), 8.09 (d, 1H), 7.94 (dd, 1H), 7.76 (d, 1H), 7.51 (dd, 1H), 7.16 (m, 5H), 6.91 (s, 2H), 3.57 (s, 3H), 3.49 (s, 6H); LCMS method A, (ES+) 532, RT=2.42 min.

### Example 50

### N-(2-(5-fluoro-2-(3,4,5-trimethoxyphenylamino)pyrimidin-4-ylamino)phenyl)thiopehene-2-sulfonamide

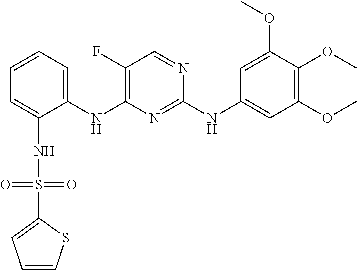

Synthesized according to the procedure of Intermediate 3a using Intermediate 2b and thiophene-2-sulfonyl chloride. ¹H NMR (CDCl₃) δ 8.55 (d, 1H), 7.87 (d, 1H), 7.57 (dd, 1H), 7.50 (dd, 1H), 7.25 (d, 1H), 7.23 (d, 1H), 7.11 (m, 2H), 6.93 (dd, 1H), 6.89 (s, 1H), 6.84 (s, 1H), 6.64 (s, 1H), 3.75 (s, 3H), 3.62 (s, 6H); LCMS method A, (ES+) 532, RT=2.44 min.

### Example 51

### N-(2-(5-fluoro-2-(3,4,5-trimethoxyphenylamino)pyrimidin-4-ylamino)phenyl)-1-methyl-1H-imidazole-4-sulfonamide

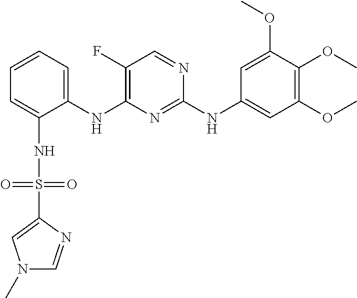

Synthesized according to the procedure of Intermediate 3a using Intermediate 2b and 1-methyl-1H-imidazole-4-sulfonyl chloride. ¹H NMR (d₆-DMSO) δ 9.30 (s, 1H), 9.04 (s, 1H), 8.42 (d, 1H), 8.05 (d, 1H), 7.43 (d, 1H), 7.28 (d, 1H), 7.18 (dd, 1H), 7.09 (s, 2H), 6.62 (t, 1H), 6.44 (t, 1H), 3.71 (s, 6H), 3.71 (s, 3H), 3.61 (s, 3H); LCMS method A, (ES+) 530, RT=2.09 min.

### Example 52

### N-(2-(5-fluoro-2-(3,4,5-trimethoxyphenylamino)pyrimidin-4-ylamino)phenyl)pyridine-3-sulfonamide

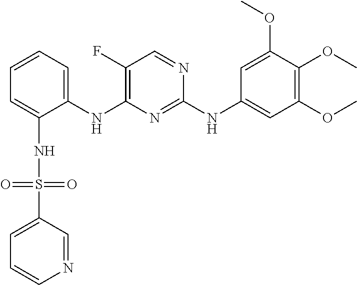

Synthesized according to the procedure of Intermediate 3a using Intermediate 2b and pyridine-3-sulfonyl chloride. ¹H NMR (d₆-DMSO) δ 10.01 (s, 1H), 8.96 (s, 1H), 8.67 (d, 1H), 8.48 (dd, 1H), 8.42 (s, 1H), 8.06 (d, 1H), 7.92 (m, 1H), 7.62 (d, 1H), 7.28 (m, 4H), 6.85 (s, 2H), 3.57 (s, 3H), 3.48 (s, 6H); LCMS method A, (ES+) 527, RT=2.24 min.

### Example 53

### N-(2-(5-fluoro-2-(3,4,5-trimethoxyphenylamino)pyrimidin-4-ylamino)phenyl)pyridine-2-sulfonamide

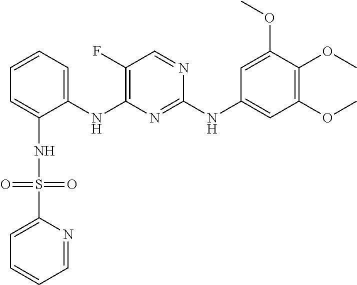

Synthesized according to the procedure of Intermediate 3a using Intermediate 2b and pyridine-2-sulfonyl chloride. ¹H NMR (d₆-DMSO) δ 10.12 (s, 1H), 8.96 (s, 1H), 8.71 (s, 1H), 8.11 (d, 1H), 7.82 (dd, 1H), 7.74 (d, 1H), 7.63 (d, 1H), 7.42 (m, 1H), 7.32 (dd, 1H), 7.15 (m, 2H), 6.87 (s, 2H), 3.56 (s, 3H), 3.48 (s, 6H); LCMS method A, (ES+) 527, RT=2.31 min.

### Example 54

### N-(3-(5-fluoro-2-(3,4,5-trimethoxyphenylamino)pyrimidin-4-ylamino)phenyl)thiophene-2-sulfonamide

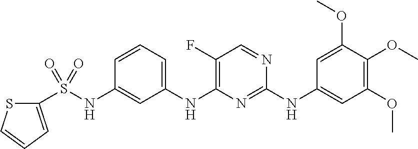

Synthesized according to the procedure of Intermediate 3a using Intermediate 2a and thiophene-2-sulfonyl chloride. ¹H NMR (d₆-DMSO) δ 10.46 (s, 1H), 9.60 (br s, 1H), 9.11 (br s, 1H), 8.12 (d, 1H), 7.89 (dd, 1H), 7.73 (d, 1H), 7.60 (dd, 1H), 7.43 (s, 1H), 7.16 (t, 1H), 7.11 (dd, 1H), 7.00 (s, 2H), 6.82 (dd, 1H), 3.59 (s, 3H), 3.58 (s, 6H); LCMS method B, (ES+) 532, RT=4.97 min.

### Example 55

### N-(3-(5-fluoro-2-(3,4,5-trimethoxyphenylamino)pyrimidin-4-ylamino)phenyl)thiophene-3-sulfonamide

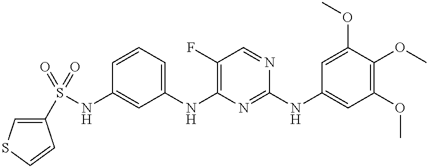

Synthesized according to the procedure of Intermediate 3a using Intermediate 2a and thiophene-3-sulfonyl chloride. ¹H NMR (d₆-DMSO) δ 10.25 (s, 1H), 9.44 (s, 1H), 8.98 (s, 1H), 8.21 (dd, 1H), 8.10 (d, 1H), 7.70 (m, 2H), 7.40 (s, 1H), 7.29 (dd, 1H), 7.14 (t, 1H), 7.02 (s, 2H), 6.80 (dd, 1H), 3.58 (s, 3H), 3.57 (s, 6H); LCMS method B, (ES+) 532, RT=4.85 min.

### Example 56

### N-(3-(5-fluoro-2-(3,4,5-trimethoxyphenylamino)pyrimidin-4-ylamino)phenyl)-1-methyl-1H-imidazole-4-sulfonamide

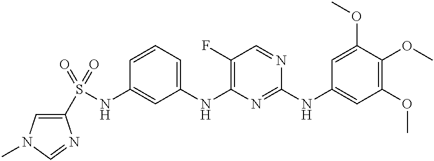

Synthesized according to the procedure of Intermediate 3a using Intermediate 2a and 1-methyl-1H-imidazole-4-sulfonyl chloride. ¹H NMR (CDCl₃) δ 8.96 (s, 1H), 7.95 (d, 1H), 7.92 (s, 1H), 7.48 (s, 1H), 7.37 (s, 1H), 7.32 (s, 1H), 7.23-7.17 (m, 2H), 7.04-7.01 (dt, 1H), 6.89 (s, 2H), 6.81 (d, 1H), 3.82 (s, 3H), 3.80 (s, 6H), 3.62 (s, 3H); LCMS method B, (ES+) 530, RT=3.79 min.

### Example 57

### N-(3-(5-fluoro-2-(3,4,5-trimethoxyphenylamino)pyrimidin-4-ylamino)phenyl)pyridine-2-sulfonamide

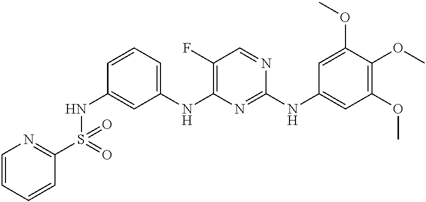

Synthesized according to the procedure of Intermediate 3a using Intermediate 2a and pyridine-2-sulfonyl chloride. ¹H NMR (d₆-DMSO) δ 10.61 (s, 1H), 9.76 (br s, 1H), 9.32 (br s, 1H), 8.70 (d, 1H), 8.14 (d, 1H), 8.05-7.98 (m, 2H), 7.62 (m, 1H), 7.58 (d, 1H), 7.40 (s, 1H), 7.10 (t, 1H), 6.92 (s, 2H), 6.85 (d, 1H), 3.59 (s, 3H), 3.54 (s, 6H); LCMS method B, (ES+) 527, RT=4.33 min.

### Example 58

### N-(3-(5-fluoro-2-(3,4,5-trimethoxyphenylamino)pyrimidin-4-ylamino)phenyl)pyridine-3-sulfonamide

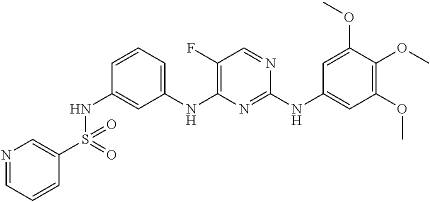

Synthesized according to the procedure of Intermediate 3a using Intermediate 2a and pyridine-3-sulfonyl chloride. ¹H NMR (d₆-DMSO) δ 10.70 (s, 1H), 10.10 (br s, 1H), 9.79 (br s, 1H), 8.95 (d, 1H), 8.78 (dd, 1H), 8.21-8.17 (m, 2H), 7.63-7.59 (m, 2H), 7.51 (s, 1H), 7.14 (t, 1H), 6.89-6.86 (m, 3H), 3.61 (s, 3H), 3.58 (s, 6H); LCMS method B, (ES+) 527, RT=4.31 min.

### Example 59

### N-(3-(5-fluoro-2-(3,4,5-trimethoxyphenylamino)pyrimidin-4-ylamino)phenyl)benzenesulfonamide hydrochloride

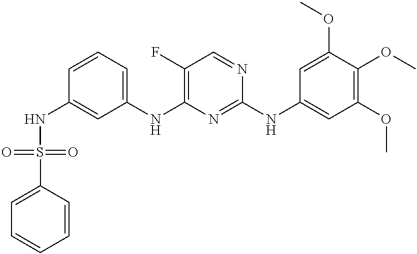

Synthesized according to the procedure of Intermediate 3a using Intermediate 2a and benzenesulfonyl chloride. ¹H NMR (d₆-DMSO) δ 10.48 (s, 1H), 10.23 (br s, 1H), 9.91 (br s, 1H), 8.22 (d, 1H), 7.82 (d, 2H), 7.61-7.53 (m, 4H), 7.45 (s, 1H), 7.12 (t, 1H), 6.87 (d, 1H), 6.84 (s, 2H), 3.62 (s, 3H), 3.56 (s, 6H); LCMS method A, (ES+) 526, RT=2.41 min.

### Example 60

### 2,2,2-trifluoro-N-(3-(5-fluoro-2-(3,4,5-trimethoxyphenylamino)pyrimidin-4-ylamino)phenyl)ethanesulfonamide

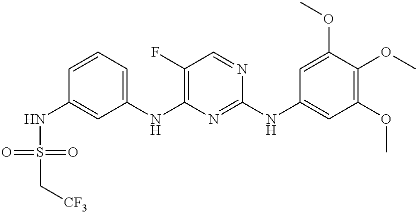

Synthesized according to the procedure of Intermediate 3a using Intermediate 2a and 2,2,2-trifluoroethanesulfonyl chloride. ¹H NMR (d₆-DMSO) δ 10.49 (s, 1H), 9.45 (s, 1H), 9.01 (s, 1H), 8.13 (d, 1H), 7.80 (d, 1H), 7.43 (s, 1H), 7.25 (t, 1H), 7.05 (s, 2H), 6.89 (d, 1H), 4.50 (d, 1H), 4.47 (d, 1H), 3.62 (s, 3H), 3.60 (s, 6H); LCMS method A, (ES+) 532, RT=2.39 min.

### Example 61

### 2-(Dimethylamino)-N-(2-(5-fluoro-2-(3,4,5-trimethoxyphenylamino)pyrimidin-4-ylamino)phenyl)ethanesulfonamide

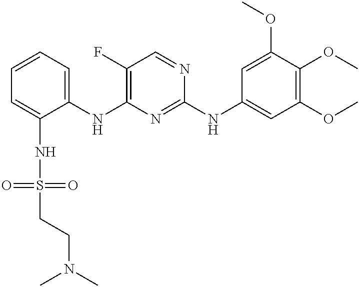

A solution of Intermediate 3a (40 mg, 0.08 mmol) and dimethylamine (0.5 mL, 2M in THF) in THF (0.5 mL) was stirred at room temperature for 2 h then water (5 mL) was added. The mixture was extracted with DCM (5 mL), washed with brine (5 mL), dried (MgSO₄), concentrated in vacuo and purified by preparative HPLC to afford 2-(dimethylamino)-N-(2-(5-fluoro-2-(3,4,5-trimethoxyphenylamino)pyrimidin-4-ylamino)phenyl)ethanesulfonamide (7.5 mg, 10%). ¹H NMR (d₆-DMSO) δ 9.06 (s, 1H), 8.74 (s, 1H), 8.14 (d, 1H), 7.75 (dd, 1H), 7.43 (d, 1H), 7.21 (m, 2H), 6.96 (s, 2H), 3.56 (s, 3H), 3.50 (s, 6H), 3.16 (t, 2H), 2.64 (t, 2H), 2.03 (s, 6H); LCMS method A, (ES+) 476, RT=1.69 min.

### Example 62

### N-(2-(5-fluoro-2-(3,4,5-trimethoxyphenylamino)pyrimidin-4-ylamino)phenyl)-2-(methylamino)ethanesulfonamide

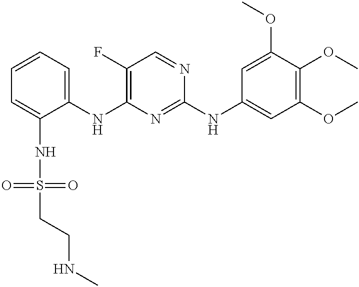

Synthesized according to the procedure in Example 61 using Intermediate 3a and methylamine. ¹H NMR (MeOD) δ 8.44 (s, 1H), 7.99 (d, 1H), 7.85 (dd, 1H), 7.47 (dd, 1H), 7.29 (m, 2H), 6.84 (s, 2H), 3.70 (s, 3H), 3.63 (s, 6H), 3.43 (t, 2H), 2.66 (s, 2H), 2.61 (s, 3H); LCMS method A, (ES+) 476, RT=1.69 min.

### Example 63

### N-(2-(5-fluoro-2-(3,4,5-trimethoxyphenylamino)pyrimidin-4-ylamino)-2-morpholinoethanesulfonamide

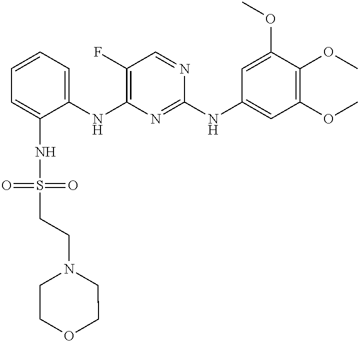

Synthesized according to the procedure in Example 61 using Intermediate 3a and morpholine. ¹H NMR (MeOD) δ 8.00 (d, 1H), 7.76 (dd, 1H), 7.52 (m, 1H), 7.30 (m, 2H), 6.86 (s, 2H), 3.70 (s, 3H), 3.61 (s, 6H), 3.56 (t, 4H), 3.24 (t, 2H), 2.75 (m, 2H), 2.34 (t, 4H); LCMS method A, (ES+) 563, RT=1.76 min.

### Example 64

### N-(3-(5-fluoro-2-(3,4,5-trimethoxyphenylamino)pyrimidin-4-ylamino)phenyl)-2-(methylamino)ethanesulfonamide

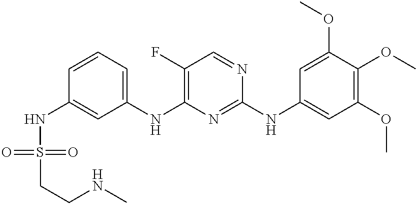

Synthesized according to the procedure in Example 61 using Intermediate 3b and methylamine. ¹H NMR (d₆-DMSO) δ 9.48 (s, 1H), 9.01 (s, 1H), 8.13 (d, 1H), 7.72 (d, 1H), 7.46 (s, 1H), 7.24 (t, 1H), 7.05 (s, 2H), 6.90 (d, 1H), 5.77 (s, 1H), 3.62 (s, 3H), 3.59 (s, 6H), 3.24 (t, 2H), 2.83 (t, 3H), 2.22 (s, 3H); LCMS method A, (ES+) 507, RT=1.65 min.

### Example 65

### 2-(Dimethylamino)-N-(3-(5-fluoro-2-(3,4,5-trimethoxyphenylamino)pyrimidin-4-ylamino)phenyl)ethanesulfonamide

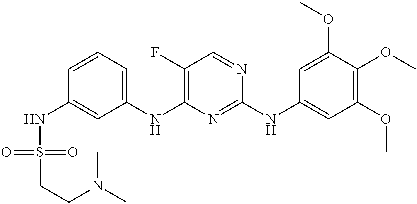

Synthesized according to the procedure in Example 61 using Intermediate 3b and dimethylamine. ¹H NMR (DMSO-d₆) δ 9.83 (br s, 1H), 9.48 (s, 1H), 9.00 (s, 1H), 8.13 (d, 1H), 7.75 (dd, 1H), 7.46 (d, 1H), 7.24 (t, 1H), 7.05 (s, 2H), 6.90 (dd, 1H), 3.63 (s, 3H), 3.59 (s, 6H), 3.26 (t, 2H), 2.63 (t, 3H), 2.08 (s, 6H); LCMS method A, (ES+) 543, RT=1.66 min.

### Example 66

### N-(2-(5-methyl-2-(3,4,5-trimethoxyphenylamino)pyrimidin-4-ylamino)phenyl)methanesulfonamide

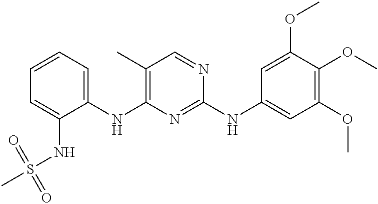

Synthesized according to the procedure in Example 1 using Intermediate 1b and 3,4,5-trimethoxyaniline. ¹H NMR (d₆-DMSO) δ 8.88 (s, 1H), 8.17 (s, 2H), 8.01 (s, 1H), 7.94 (s, 1H), 7.37 (d, 1H), 7.26 (t, 1H), 7.16 (t, 1H), 7.16 (t, 1H), 7.00 (s, 2H), 3.56 (s, 3H), 3.53 (s, 6H), 2.92 (s, 3H), 2.11 (s, 3H); LCMS method A, (ES+) 460, RT=2.28 min.

### Example 67

### N-(2-(2-(3,5-dimethylphenylamino)-5-methylpyrimidin-4-ylamino)phenyl)methanesulfonamide

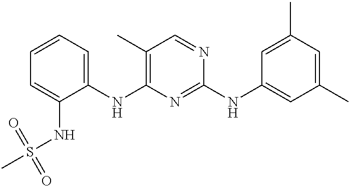

Synthesized according to the procedure in Example 1 using Intermediate 1b and 3,5-dimethylaniline. ¹H NMR (d₆-DMSO) δ 10.41 (s, 1H), 9.65 (s, 1H), 9.31 (s, 1H), 7.95 (s, 1H), 7.55 (d, 1H), 7.46 (d, 1H), 7.42 (t, 1H), 7.30 (t, 1H), 6.87 (s, 2H), 6.65 (s, 1H), 2.87 (s, 3H), 2.19 (s, 3H), 2.07 (s, 6H); LCMS method A, (ES+) 398, RT=2.00 min.

### Example 68

### N-(2-(5-nitro-2-(3,4,5-trimethoxyphenylamino)pyrimidin-4-ylamino)phenyl)methanesulfonamide

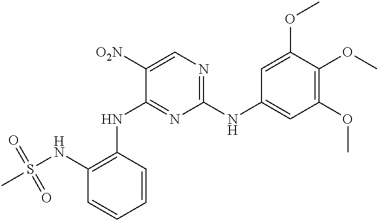

Synthesized according to the procedure in Example 1 using Intermediate if and 3,4,5-trimethoxyaniline. ¹H NMR (d₆-DMSO) δ 10.58 (s, 1H), 10.35 (s, 1H), 9.38 (s, 1H), 9.13 (s, 1H), 7.42 (m, 2H), 7.29 (m, 2H), 6.83 (s, 2H), 3.61 (s, 3H), 3.46 (s, 6H), 2.97 (s, 3H); LCMS method A, (ES+) 491, RT=2.55 min.

### Example 69

### N-(2-(2-(3,4,5-trimethoxyphenylamino)pyrimidin-4-ylamino)phenyl)methanesulfonamide

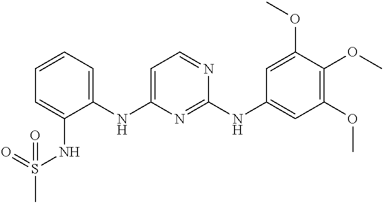

Synthesized according to the procedure in Example 1 using Intermediate 1d and 3,4,5-trimethoxyaniline. ¹H NMR (MeOD) δ 7.97 (d, 1H), 7.63 (dd, 1H), 7.42 (dd, 1H), 7.27-7.22 (m, 2H), 6.88 (s, 2H), 6.22 (d, 1H), 3.71 (s, 3H), 3.65 (s, 6H), 2.87 (s, 3H); LCMS method A, (ES+) 446, RT=1.38 min.

### Example 70

### N-(2-(5-bromo-2-(3,4,5-trimethoxyphenylamino)pyrimidin-4-ylamino)phenyl)methanesulfonamide

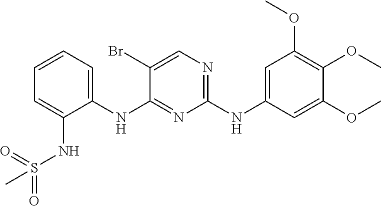

Synthesized according to the procedure in Example 1 using Intermediate 1c and 3,4,5-trimethoxyaniline. ¹H NMR (d₆-DMSO) δ 9.34 (br s, 1H), 9.25 (s, 1H), 8.42 (s, 1H), 8.26 (s, 1H), 8.02 (br s, 1H), 7.37 (dd, 1H), 7.35-7.15 (m, 2H), 6.94 (s, 2H), 3.58 (s, 3H), 3.54 (s, 6H), 2.96 (s, 3H); LCMS method A, (ES+) 524, 526, RT=2.36 min.

### Example 71

### N-(2-(5-fluoro-2-(3,4,5-trimethoxyphenylamino)pyrimidin-4-ylamino)phenyl)-N-methylmethanesulfonamide

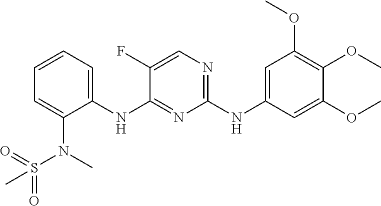

Synthesized according to the procedure in Example 1 using Intermediate 1e and 3,4,5-trimethoxyaniline. ¹H NMR (CDCl₃) δ 8.39 (d, 1H), 8.08 (br s, 1H), 7.96 (br s, 1H), 7.26-7.34 (m, 3H), 7.13-7.15 (m, 1H), 6.78 (s, 2H), 3.82 (s, 3H), 3.75 (s, 6H), 3.28 (s, 3H), 2.97 (s, 3H); LCMS method A, (ES+) 474, RT=2.52 min.

### Example 72

### N-(2-(2-(3,5-dimethylphenylamino)-5-fluoropyrimidin-4-ylamino)phenyl)-N-methylmethanesulfonamide

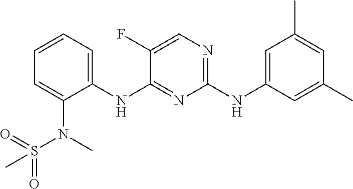

Synthesized according to the procedure in Example 1 using Intermediate 1e and 3,5-dimethylaniline. ¹H NMR (CDCl₃) δ 8.44 (d, 1H), 8.07 (br s, 1H), 7.98 (br s, 1H), 7.31-7.41 (m, 2H), 7.14-7.18 (m, 3H), 7.01 (br s, 1H), 6.67 (br s, 1H), 3.29 (s, 3H), 2.99 (s, 3H), 2.28 (s, 6H); LCMS method A, (ES+) 416, RT=2.61 min.

### Example 73

### N-(2-(5-fluoro-2-(3-methoxy-4-methylphenylamino)pyrimidin-4-ylamino)phenyl)methanesulfonamide

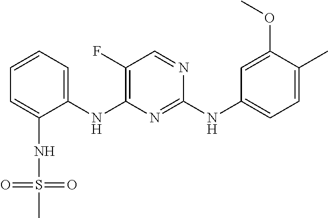

Synthesized according to the procedure in Example 1 using Intermediate 1a and the appropriate aniline derivative. ¹H NMR (d₆-DMSO) δ 10.13 (s, 1H), 9.98 (s, 1H), 9.33 (s, 1H), 8.28 (d, 1H), 7.57 (d, 1H), 7.51 (d, 1H), 7.36 (t, 1H), 7.27 (t, 1H), 6.96 (s, 1H), 6.91 (dd, 2H), 3.50 (s, 3H), 2.94 (s, 3H), 2.05 (s, 3H); LCMS method A, (ES+) 418, RT=2.35 min.

### Example 74

### N-(2-(2-(3,4-dimethoxy-5-(2-(pyrrolidin-1-yl)ethoxy)phenylamino)-5-fluoropyrimidin-4-ylamino)phenyl)methanesulfonamide

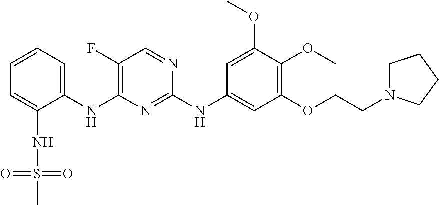

Synthesized according to the procedure in Example 1 using Intermediate 1a and the appropriate aniline derivative. ¹H NMR (MeOD) δ 7.90 (br s, 1H), 7.86 (d, 1H), 7.36 (d, 1H), 7.23-7.13 (m, 2H), 7.04 (s, 1H), 7.03 (s, 1H), 3.91 (t, 2H), 3.66 (s, 3H), 3.57 (s, 3H), 3.04 (br s, 2H), 2.89 (m, 4H), 2.85 (s, 3H), 1.85 (m, 4H); LCMS method B, (ES+) 561, RT=4.99 min.

### Example 75

### N-(2-(2-(3,5-dimethoxy-4-(2-(pyrrolidin-1-yl)ethoxy)phenylamino)-5-fluoropyrimidin-4-ylamino)phenyl)methanesulfonamide

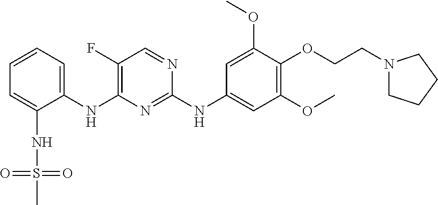

Synthesized according to the procedure in Example 1 using Intermediate 1a and the appropriate aniline derivative. ¹H NMR (d₆-DMSO) δ 9.07 (s, 1H), 8.78 (s, 1H), 8.12 (d, 1H), 7.92 (d, 1H), 7.36 (dd, 1H), 7.15-7.08 (m, 2H), 7.00 (s, 2H), 3.85 (t, 2H), 3.56 (s, 6H), 2.86 (s, 3H), 2.81 (t, 2H), 2.65 (m, 4H), 1.72 (m, 4H); LCMS method B, (ES+) 547, RT=4.94 min.

### Example 76

### N-(2-(5-fluoro-2-(3-hydroxyphenylamino)pyrimidin-4-ylamino)phenyl)methanesulfonamide

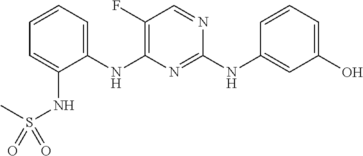

Synthesized according to the procedure in Example 1 using Intermediate 1a and the appropriate aniline derivative. ¹H NMR: (d₆-DMSO) δ 10.37 (br s, 1H), 10.25 (br s, 1H), 9.36 (s, 1H), 8.34 (d, 1H), 7.58 (d, 1H), 7.52 (d, 1H), 7.37 (t, 1H), 7.30 (t, 1H), 6.89 (t, 1H), 6.82 (d, 1H), 6.73 (s, 1H), 6.44 (d, 1H), 2.94 (s, 3H); LCMS method A, (ES+) 390, RT=1.88 min.

### Example 77

### N-(2-(5-fluoro-2-(3-(2-morpholinoethoxy)phenylamino)pyrimidin-4-ylamino)phenyl)methanesulfonamide

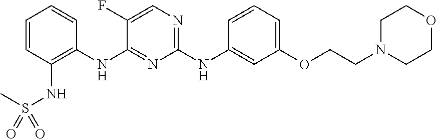

Synthesized according to the procedure in Example 1 using Intermediate 1a and the appropriate aniline derivative. ¹H NMR: (d₆-DMSO) δ 9.16 (s, 1H), 8.68 (s, 1H), 8.15 (s, 3H), 7.85 (d, 1H), 7.86 (d, 1H), 7.44 (d, 1H), 7.28 (m, 3H), 7.15 (d, 1H), 7.02 (t, 1H), 6.45 (d, 1H), 3.92 (t, 2H), 3.58 (t, 2H), 2.93 (s, 3H), 2.66 (t, 2H), 2.46 (m, 4H); LCMS method A, (ES+) 503, RT=1.63 min.

### Example 78

### N-(2-(5-fluoro-2-(3-(2-hydroxyethoxy)phenylamino)pyrimidin-4-ylamino)phenyl)methanesulfonamide

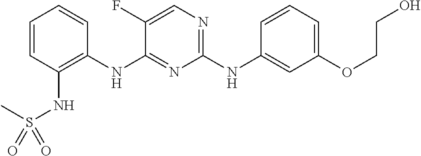

Synthesized according to the procedure in Example 1 using Intermediate 1a and the appropriate aniline derivative. ¹H NMR: (d₆-DMSO) δ 10.28 (br s, 1H), 10.05 (br s, 1H), 9.33 (s, 1H), 8.33 (d, 1H), 7.59 (d, 1H), 7.52 (d, 1H), 7.34 (m, 2H), 7.05 (t, 1H), 7.00 (s, 1H), 6.92 (d, 1H), 6.58 (d, 1H), 3.79 (t, 2H), 3.69 (t, 2H), 2.94 (s, 3H); LCMS method A, (ES+) 434, RT=1.95 min.

### Example 79

### N-(2-(5-fluoro-2-(3-(2-(piperidin-1-yl)ethoxy)phenylamino)pyrimidin-4-ylamino)phenyl)methanesulfonamide formate

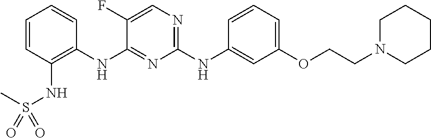

Synthesized according to the procedure in Example 1 using Intermediate 1a and the appropriate aniline derivative. ¹H NMR: (d₆-DMSO) δ 9.16 (s, 1H), 8.71 (s, 1H), 8.19 (s, 2H), 8.14 (d, 1H), 7.87 (d, 1H), 7.43 (d, 1H), 7.30-7.15 (m, 4H), 7.03 (t, 1H), 6.45 (d, 1H), 3.95 (t, 2H), 2.92 (s, 3H), 2.72 (t, 2H), 1.53 (m, 4H), 1.40 (m, 2H); LCMS method A, (ES+) 501, RT=2.02 min.

### Example 80

### 3-(5-fluoro-4-(2-(methylsulfonamido)phenylamino)pyrimidin-2-ylamino)-N-methylbenzamide

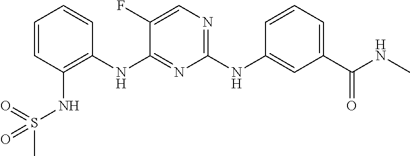

Synthesized according to the procedure in Example 1 using Intermediate 1a and the appropriate aniline derivative. ¹H NMR (d₆-DMSO) δ 9.32 (br s, 1H), 8.69 (br s, 1H), 8.26 (d, 1H), 8.15-8.16 (m, 2H), 7.96 (t, 1H), 7.92 (d, 1H), 7.65 (d, 1H), 7.42 (d, 1H), 7.18-7.29 (m, 4H), 2.92 (s, 3H), 2.76 (d, 2H); LCMS method A, (ES+) 431, RT=2.18 min.

### Example 81

### N,N-diethyl-3-(5-fluoro-4-(2-(methylsulfonamido)phenylamino)pyrimidin-2-ylamino)benzamide

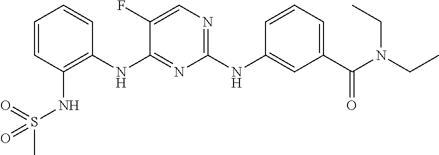

Synthesized according to the procedure in Example 1 using Intermediate 1a and the appropriate aniline derivative. ¹H NMR (d₆-DMSO) δ 9.35 (br s, 1H), 8.73 (br s, 1H), 8.15 (d, 1H), 7.85 (d, 1H), 7.62-7.64 (m, 2H), 7.43 (d, 1H), 7.16-7.27 (m, 3H), 6.79 (d, 2H), 3.41 (br s, 2H), 3.12 (br s, 2H), 2.91 (s, 3H), 1.05 (br d, 6H); LCMS method A, (ES+) 473, RT=2.35 min.

### Example 82

### N-(2-(5-fluoro-2-(3-(pyrrolidine-1-carbonyl)phenylamino)pyrimidin-4-ylamino)phenyl)methanesulfonamide

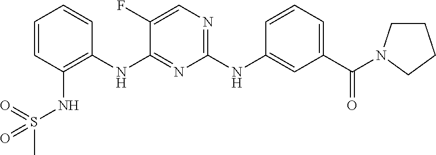

Synthesized according to the procedure in Example 1 using Intermediate 1a and the appropriate aniline derivative. ¹H NMR (d₆-DMSO) δ 9.34 (br s, 1H), 8.77 (br s, 1H), 8.21 (d, 1H), 7.81-7.83 (m, 1H), 7.66 (s, 1H), 7.62 (d, 1H), 7.42-7.43 (m, 1H), 7.25-7.29 (m, 3H), 7.68 (d, 1H), 2.97 (d, 2H), 2.91 (s, 3H), 2.89 (d, 2H), 0.91-1.09 (m, 4H); LCMS method A, (ES+) 471, RT=2.36 min.

### Example 83

### N-cyclopropyl-3-(5-fluoro-4-(2-(methylsulfonamido)phenylamino)pyrimidin-2-ylamino)benzamide

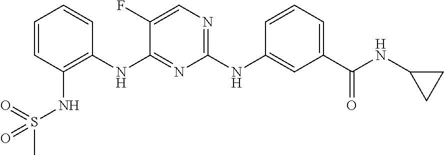

Synthesized according to the procedure in Example 1 using Intermediate 1a and the appropriate aniline derivative. ¹H NMR (d₆-DMSO) δ 9.33 (br s, 1H), 8.68 (br s, 1H), 8.31 (d, 1H), 8.16 (d, 1H), 7.90-7.92 (m, 2H), 7.76 (d, 1H), 7.43 (d, 2H), 7.16-7.27 (m, 4H), 2.93 (s, 3H), 2.83 (m, 1H), 0.66-0.68 (m, 2H), 0.53-0.55 (m, 2H); LCMS method A, (ES+) 457, RT=2.28 min.

### Example 84

### 3-(5-fluoro-4-(2-(methylsulfonamido)phenylamino)pyrimidin-2-ylamino)-N,N-dimethylbenzamide

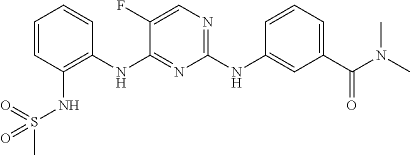

Synthesized according to the procedure in Example 1 using Intermediate 1a and the appropriate aniline derivative. ¹H NMR (d₆-DMSO) δ 9.36 (br s, 1H), 8.75 (br s, 1H), 8.18 (d, 1H), 7.81-7.83 (m, 1H), 7.66 (s, 1H), 7.59 (d, 1H), 7.42-7.44 (m, 1H), 7.23-7.25 (m, 3H), 7.68 (d, 1H), 2.96 (s, 3H), 2.91 (s, 3H), 2.82 (s, 3H); LCMS method A, (ES+) 445, RT=2.34 min.

### Example 85

### N-(2-(5-fluoro-2-(3-(pyrrolidin-1-yl)phenylamino)pyrimidin-4-ylamino)phenyl)methanesulfonamide

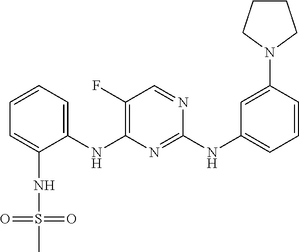

Synthesized according to the procedure in Example 1 using Intermediate 1a and the appropriate aniline derivative. ¹H NMR (d₆-DMSO) δ 10.26-10.31 (m, 2H), 9.34 (s, 1H), 8.32 (d, 1H), 7.52 (d, 2H), 7.37 (t, 1H), 7.26 (t, 1H), 6.98 (t, 1H), 6.55-6.59 (m, 2H), 6.29-6.31 (m, 1H), 2.98-3.00 (m, 4H), 2.93 (s, 3H), 1.90 (m, 4H); LCMS method A, (ES+) 443, RT=2.36 min.

### Example 86

### N-(2-(5-fluoro-2-(4-(2-morholinoethoxy)phenylamino)pyrimidin-4-ylamino)phenyl)methanesulfonamide

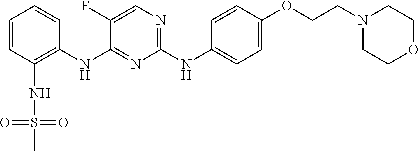

Synthesized according to the procedure in Example 1 using Intermediate 1a and the appropriate aniline derivative. ¹H NMR (d₆-DMSO) δ 9.00 (s, 1H), 8.62 (s, 1H), 8.14 (s, 1H), 8.09 (d, 1H), 7.86 (dd, 1H), 7.44-7.46 (m, 3H), 7.31-7.32 (m, 1H), 7.25-7.27 (m, 1H), 6.74 (d, 2H), 4.01 (t, 2H), 3.57-3.59 (m, 4H), 2.92 (s, 3H), 2.68 (t, 2H), 2.46-2.50 (m, 4H); LCMS method A, (ES+) 503, RT=1.42 min.

### Example 87

### N-(2-(5-fluoro-2-(4-(piperidin-1-yl)ethoxy)phenylamino)pyrimidin-4-ylamino)phenyl)methanesulfonamide

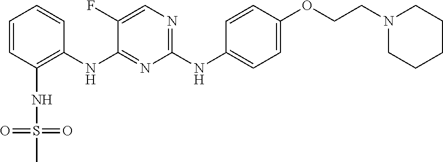

Synthesized according to the procedure in Example 1 using Intermediate 1a and the appropriate aniline derivative. ¹H NMR (d₆-DMSO) δ 9.01 (s, 1H), 8.64 (s, 1H), 8.18 (s, 1H), 8.09 (d, 1H), 7.87 (dd, 1H), 7.42-7.47 (m, 3H), 7.29-7.33 (m, 1H), 7.22-7.26 (m, 1H), 6.75 (d, 2H), 4.03 (t, 2H), 2.92 (s, 3H), 2.75 (t, 2H), 2.56 (m, 4H), 1.53-1.56 (m, 4H), 1.40-1.41 (m, 2H); LCMS method A, (ES+) 501, RT=1.54 min.

### Example 88

### N-(2-(5-fluoro-2-(3-methoxy-4-(pyrrolidin-1-yl)phenylamino)pyrimidin-4-ylamino)phenyl)methanesulfonamide

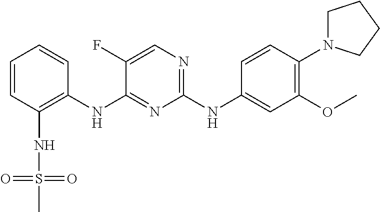

Synthesized according to the procedure in Example 1 using Intermediate 1a and the appropriate aniline derivative. ¹H NMR (d₆-DMSO) δ 9.97 (s, 1H), 9.42-9.45 (m, 1H), 9.35 (s, 1H), 8.27 (d, 1H), 7.66 (d, 1H), 7.55 (d, 1H), 7.47-7.50 (m, 2H), 7.29-7.31 (m, 2H), 7.16 (d, 1H), 3.62 (m, 5H), 3.44-3.47 (m, 2H), 2.94 (s, 3H), 2.06 (m, 4H); LCMS method A, (ES+) 473, RT=1.67 min.

### Example 89

### N-(2-(5-Fluoro-2-(4-(2-(4-methylpiperazin-1-yl)ethoxy)phenylamino)pyrimidin-4-ylamino)phenyl)methanesulfonamide

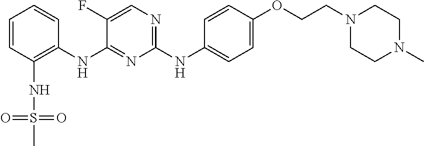

Synthesized according to the procedure in Example 1 using Intermediate 1a and the appropriate aniline derivative. ¹H NMR (d₆-DMSO) δ 9.00 (s, 1H), 8.66 (s, 1H), 8.08 (d, 1H), 7.91 (d, 1H), 7.46 (d, 2H), 7.40 (d, 1H), 7.18-7.27 (m, 2H), 6.75 (d, 2H), 4.01 (t, 2H), 3.34 (m, 4H), 2.89 (s, 3H), 2.65 (t, 2H), 2.33-2.34 (m, 4H), 2.17 (s, 3H); LCMS method A, (ES+)=516, RT=1.43 min.

### Example 90

### N-(2-(5-fluoro-2-(4-(3-piperidin-1-yl)propoxy)phenylamino)pyrimidin-4-ylamino)phenyl)methanesulfonamide

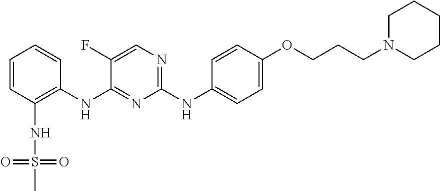

Synthesized according to the procedure in Example 1 using Intermediate 1a and the appropriate aniline derivative. ¹H NMR (d₆-DMSO) δ 9.00 (s, 1H), 8.67 (s, 1H), 8.08 (d, 1H), 7.92 (dd, 1H), 7.46 (d, 2H), 7.40 (dd, 1H), 7.19-7.24 (m, 2H), 6.74 (d, 2H), 3.92 (t, 2H), 2.89 (s, 3H), 2.43-2.48 (m, 6H), 1.84-1.87 (m, 2H), 1.51-1.52 (m, 4H), 1.40-1.41 (m, 2H); LCMS method A, (ES+)=515, RT=1.59 min.

### Example 91

### N-(2-(5-fluoro-2-(4-(3-(4-methylpiperazin-1-yl)propoxy)phenylamino)pyrimidin-4-ylamino)phenyl)methanesulfonamide

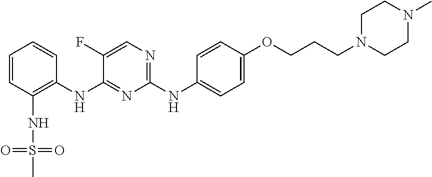

Synthesized according to the procedure in Example 1 using Intermediate 1a and the appropriate aniline derivative. ¹H NMR (d₆-DMSO) δ 9.00 (s, 1H), 8.64 (s, 1H), 8.08 (d, 1H), 7.90 (dd, 1H), 7.41-7.46 (m, 3H), 7.21-7.27 (m, 2H), 6.73 (d, 2H), 3.92 (t, 2H), 2.90 (s, 3H), 2.35-2.43 (m, 10H), 2.18 (s, 3H), 1.83-1.84 (m, 2H); LCMS method A, (ES+) 530, RT=1.39 min.

### Example 92

### N-(2-(5-fluoro-2-(4-(3-pyrrolidin-1-yl)propoxy)phenylamino)pyrimidin-4-ylamino)phenyl)methanesulfonamide

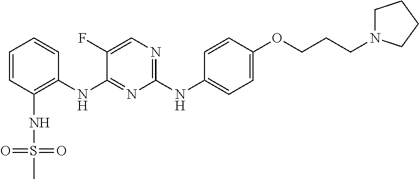

Synthesized according to the procedure in Example 1 using Intermediate 1a and the appropriate aniline derivative. ¹H NMR (d₆-DMSO) δ 9.00 (s, 1H), 8.68 (s, 1H), 8.08 (d, 1H), 7.93 (dd, 1H), 7.46 (d, 2H), 7.40 (dd, 1H), 7.18-7.22 (m, 2H), 6.74 (d, 2H), 3.94 (t, 2H), 2.88 (s, 3H), 2.65 (t, 2H), 2.50-2.58 (m, 4H), 1.87-1.90 (m, 2H), 1.73-1.74 (m, 4H); LCMS, method A, (ES+) 501, RT=1.55 min.

### Example 93

### N-(2-(5-fluoro-2-(4-(2-pyrrolidin-1-yl)ethylamino)phenylamino)pyrimidin-4-ylamino)phenyl)methanesulfonamide formate

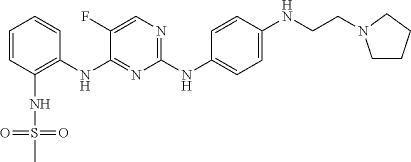

Synthesized according to the procedure in Example 1 using Intermediate 1a and the appropriate aniline derivative. ¹H NMR (d₆-DMSO) δ 8.75 (s, 1H), 8.58 (s, 1H), 8.03 (d, 1H), 7.94 (dd, 1H), 7.39 (dd, 1H), 7.18-7.28 (m, 4H), 6.45 (d, 2H), 3.17 (t, 2H), 2.90 (s, 3H), 2.85 (t, 2H), 2.78-2.79 (m, 4H), 1.76-1.79 (m, 4H); LCMS method A, (ES+) 486, RT=1.34 min.

### Example 94

### N-(2-(5-fluoro-2-(3-methoxy-5-(2-(pyrrolidin-1-yl)ethoxy)phenylamino)pyrimidin-4-ylamino)phenyl)methanesulfonamide formate salt

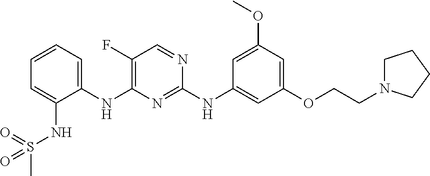

Synthesized according to the procedure in Example 1 using Intermediate 1a and the appropriate aniline derivative. ¹H NMR (d₆-DMSO) δ 9.13 (s, 1H), 8.71 (s, 1H), 8.16 (s, 2H), 8.15 (d, 1H), 7.89 (d, 1H), 7.41 (dd, 1H), 7.15-7.30 (m, 2H), 6.89 (d, 2H), 6.04 (t, 1H), 3.91 (t, 2H), 3.58 (s, 3H), 2.91 (s, 3H), 2.85 (t, 2H), 2.55-2.65 (m, 4H), 1.60-1.75 (m, 4H); LCMS method A, (ES+) 517, RT=1.67 min.

### Example 95

### N-(2-(5-fluoro-2-(3-(2-(pyrrolidin-1-yl)ethoxy)phenylamino)pyrimidin-4-ylamino)phenyl)methanesulfonamide

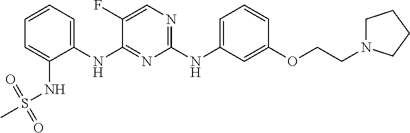

Synthesized according to the procedure in Example 1 using Intermediate 1a and the appropriate aniline derivative. ¹H NMR (d₆-DMSO) δ 9.25 (br s, 1H), 9.16 (s, 1H), 8.71 (s, 1H), 8.14 (d, 1H), 7.90 (d, 1H), 7.42 (dd, 1H), 7.30-7.15 (m, 4H), 7.04 (t, 1H), 6.46 (dd, 1H), 3.94 (t, 2H), 2.90 (s, 3H), 2.81 (t, 2H), 2.57 (m, 4H), 1.71 (m, 4H); LCMS method C, (ES+) 487 RT=2.32 min.

### Example 96

### N-(2-(5-fluoro-2-(4-(2-(pyrrolidin-1-yl)ethoxy)phenylamino)pyrimidin-4-ylamino)phenyl)methanesulfonamide

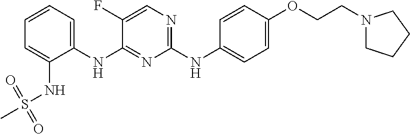

Synthesized according to the procedure in Example 1 using Intermediate 1a and the appropriate aniline derivative. ¹H NMR (d₆-DMSO) δ 9.23 (br s, 1H), 9.01 (s, 1H), 8.66 (s, 1H), 8.08 (d, 1H), 7.90 (dd, 1H), 7.47 (d, 2H), 7.41 (dd, 1H), 7.26-7.19 (m, 2H), 6.76 (d, 2H), 4.01 (t, 2H), 2.90 (s, 3H), 2.83 (t, 2H), 2.59 (m, 4H), 1.71 (m, 4H); LCMS method B, (ES+) 487 RT=4.44 min.

### Example 97

### N-(2-(5-fluoro-2-(3-((1-methylpiperidin-2-yl)methoxy)phenylamino)pyrimidin-4-ylamino)phenyl)methanesulfonamide

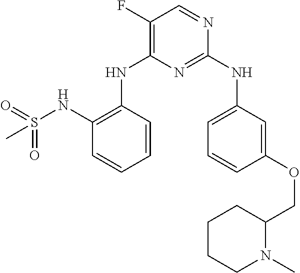

Synthesized according to the procedure in Example 1 using Intermediate 1a and the appropriate aniline derivative. ¹H NMR (d₆-DMSO) δ 9.18 (s, 1H), 8.74 (s, 1H), 8.17 (s, 1H), 8.14-8.17 (m, 2H), 7.91 (d, 1H), 7.42 (dd, 1H), 7.28 (s, 1H), 7.19-7.24 (m, 2H), 7.14-7.18 (m, 1H), 7.03 (t, 1H), 6.43 (dd, 1H), 3.66-3.75 (m, 2H), 2.90 (s, 3H), 2.85-2.90 (m, 1H), 2.67-2.75 (m, 1H), 2.24 (s, 3H), 1.95-2.03 (m, 2H), 1.80-1.90 (m, 1H), 1.64-1.73 (m, 2H), 1.46-1.54 (m, 1H), 0.97-1.09 (m, 1H); LCMS method B, (ES+) 501, RT=5.32 min.

### Example 98

### N-(2-(5-fluoro-2-(3-((1-methylpiperidin-3-yl)methoxy)phenylamino)pyrimidin-4-ylamino)phenyl)methanesulfonamide

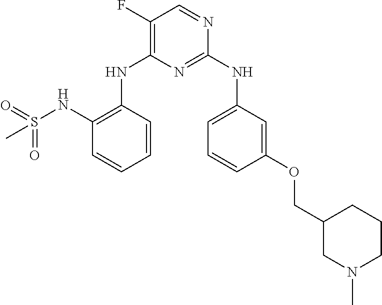

Synthesized according to the procedure in Example 1 using Intermediate 1a and the appropriate aniline derivative. ¹H NMR (d₆-DMSO) δ 9.18 (s, 1H), 8.74 (s, 1H), 8.17 (s, 1H), 8.14 (d, 1H), 7.91 (dd, 1H), 7.42 (dd, 1H), 7.28 (s, 1H), 7.22-7.26 (m, 1H), 7.14-7.18 (m, 1H), 7.03 (t, 1H), 6.42 (dd, 1H), 3.73-3.76 (m, 2H), 2.90 (s, 3H), 2.84-2.85 (m, 1H), 2.70-2.76 (m, 1H), 2.24 (s, 3H), 1.97-2.03 (m, 2H), 1.83-1.88 (m, 1H), 1.64-1.72 (m, 2H), 1.47-1.56 (m, 1H), 0.97-1.09 (m, 1H); LCMS method B, (ES+) 501, RT=5.31 min.

### Example 99

### 2-(3-(5-fluoro-4-(2-(methylsulfonamido)phenylamino)pyrimidin-2-ylamino)phenoxy)acetic acid hydrochloride

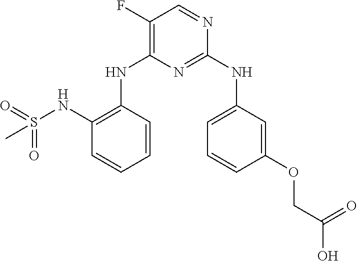

2-(3-(5-fluoro-4-(2-(methylsulfonamido)phenylamino)pyrimidin-2-ylamino)phenoxy)acetic acid ethyl ester, prepared according to the procedure in Example 1 using Intermediate 1a, was dissolved in THF and treated with 1M KOH in 6:1 methanol-water at 50° C. for 3 h. The mixture was concentrated in vacuo and acidified with hydrochloric acid. The aqueous layer was extracted with DCM, the organics were dried and concentrated to afford 2-(3-(5-fluoro-4-(2-(methylsulfonamido)phenylamino)pyrimidin-2-ylamino)phenoxy)acetic acid hydrochloride. ¹H NMR (d₆-DMSO) δ 9.36 (br s, 1H), 9.26 (s, 1H), 8.96 (br s, 1H), 8.17 (d, 1H), 7.81 (d, 1H), 7.45 (dd, 2H), 7.33 (dt, 1H), 7.26 (dt, 1H), 7.16-7.20 (m, 2H), 7.04 (t, 1H), 6.44 (dm, 1H), 4.54 (s, 2H), 2.94 (s, 3H); LCMS method B, (ES+) 448, RT=6.79 min.

### Example 100

### N,N-diethyl-2-(3-(5-fluoro-4-(2-(methylsulfonamido)phenylamino)pyrimidin-2-ylamino)phenoxy)acetamide 2,2,2-trifluoroacetate

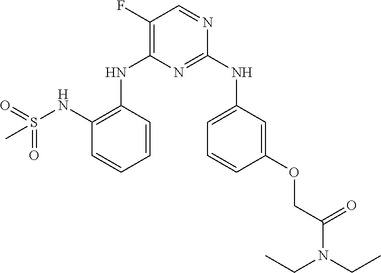

Synthesized according to the procedure in Example 1 using Intermediate 1a and the appropriate aniline derivative. ¹H NMR (d₆-DMSO) δ 9.30 (s, 1H), 9.25 (s, 1H), 8.84 (s, 1H), 8.15 (d, 1H), 7.83 (d, 1H), 7.44 (dd, 1H), 7.33 (dt, 1H), 7.22-7.27 (m, 2H), 7.15 (d, 1H), 7.03 (t, 1H), 6.43 (dd, 1H), 4.63 (s, 2H), 3.29 (m, 4H), 2.94 (s, 3H), 1.13 (t, 3H), 1.03 (t, 3H); LCMS method A, (ES+) 503, RT=2.25 min.

### Example 101

### N-ethyl-2-(3-(5-fluoro-4-(2-(methylsulfonamido)phenylamino)pyrimidin-2-ylamino)phenoxy)acetamide

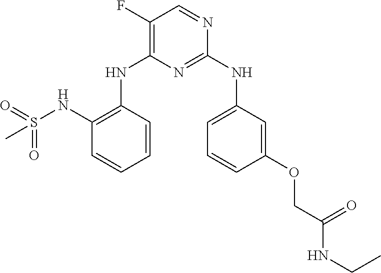

Synthesized according to the procedure in Example 1 using Intermediate 1a and the appropriate aniline derivative. ¹H NMR (d₆-DMSO) δ 9.24 (s, 2H), 8.73 (s, 1H), 8.15 (d, 1H), 8.03 (t, 1H), 7.85 (d, 1H), 7.44 (dd, 1H), 7.30-7.32 (m, 2H), 7.20-7.26 (m, 2H), 7.05 (t, 1H), 6.46 (dd, 1H), 4.31 (s, 2H), 3.14-3.17 (m, 2H), 2.94 (s, 3H), 1.04 (t, 3H); LCMS method A, (ES+) 475, RT=2.08 min.

### Example 102

### N-(2-(5-bromo-2-(phenylamino)pyrimidin-4-ylamino)phenyl)methanesulfonamide hydrochloride

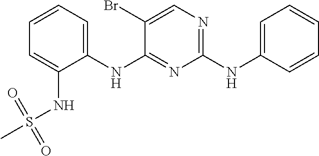

Synthesized according to the procedure in Example 1 using Intermediate 1c and the appropriate aniline derivative. ¹H NMR (d₆-DMSO) δ 10.02 (br s, 1H), 9.33 (s, 1H), 9.12 (br s, 1H), 8.37 (s, 1H), 7.76 (m, 1H), 7.47 (dd, 1H), 7.40-7.34 (m, 4H), 7.14 (t, 2H), 6.96 (t, 1H), 2.93 (s, 3H); LCMS method A, (ES+) 434, 436, RT=2.47 min.

### Example 103

### N-(2-(5-bromo-2-(3-(difluoromethoxy)phenylamino)pyrimidin-4-ylamino)phenyl)methanesulfonamide

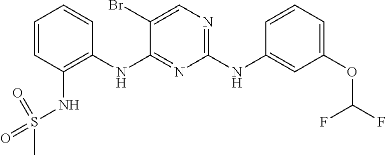

Synthesized according to the procedure in Example 1 using Intermediate 1c and the appropriate aniline derivative. LCMS method A, (ES+) 500, 502 RT=2.79 min.

### Example 104

### N-(2-(5-bromo-2-(3-methoxy-4-methylphenylamino)pyrimidin-4-ylamino)phenyl)methanesulfonamide

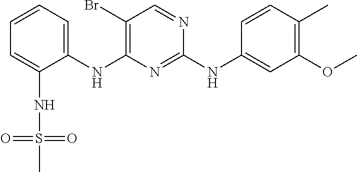

Synthesized according to the procedure in Example 1 using Intermediate 1c and the appropriate aniline derivative. ¹H NMR (d₆-DMSO) δ 9.86 (br s, 1H), 9.34 (s, 1H), 8.34 (s, 1H), 7.80-7.82 (m, 1H), 7.30-7.33 (m, 2H), 6.88-6.99 (m, 1H), 2.96 (s, 3H), 2.06 (s, 3H); LCMS method A, (ES+) 479, RT=2.25 min.

### Example 105

### N-(2-(5-bromo-2-(3,5-dimethoxyphenylamino)pyrimidin-4-ylamino)phenyl)methanesulfonamide

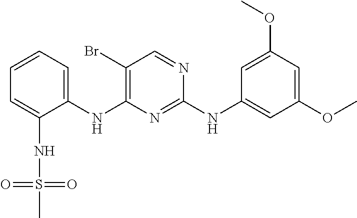

Synthesized according to the procedure in Example 1 using Intermediate 1c and the appropriate aniline derivative. ¹H NMR (d₆-DMSO) δ 9.64 (br s, 1H), 9.34 (s, 1H), 8.76 (s, 1H), 8.33 (s, 1H), 7.92-7.94 (m, 1H), 7.39-7.42 (m, 1H), 7.24-7.33 (m, 2H), 6.75 (s, 1H), 6.13 (m, 1H), 3.60 (s, 3H), 2.96 (s, 3H); LCMS method A, (ES+) 460, RT=2.20 min.

### Example 106

### N-(2-(5-bromo-2-(4-methoxyphenylamino)pyrimidin-4-ylamino)phenyl)methanesulfonamide

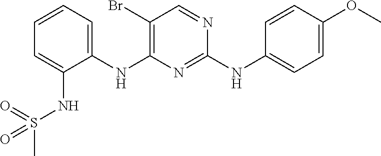

Synthesized according to the procedure in Example 1 using Intermediate 1c and the appropriate aniline derivative. ¹H NMR (d₆-DMSO) δ 9.03 (br s, 1H), 9.21 (s, 1H), 8.40 (s, 1H), 8.21 (s, 1H), 8.02 (br s, 1H), 7.33-7.44 (m, 4H), 6.75 (d, 2H), 3.70 (s, 3H), 2.96 (s, 3H); LCMS method A, (ES+) 465, RT=2.10 min.

### Example 107

### N-(2-(5-bromo-2-(3,4-dimethoxyphenylamino)pyrimidin-4-ylamino)phenyl)methanesulfonamide

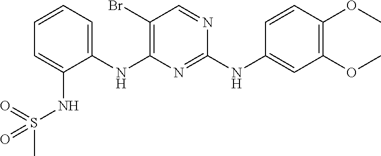

Synthesized according to the procedure in Example 1 using Intermediate 1c and the appropriate aniline derivative. ¹H NMR (d₆-DMSO) δ 9.32 (br s, 1H), 9.18 (s, 1H), 8.40 (s, 1H), 8.23 (s, 1H), 8.04 (br s, 1H), 7.38 (d, 1H), 7.31 (t, 1H), 7.22 (t, 1H), 7.17 (br s, 1H), 7.10 (d, 1H), 6.75 (d, 1H), 3.70 (s, 3H), 3.55 (s, 3H), 2.96 (s, 3H); LCMS method A, (ES+) 495, RT=2.30 min.

### Example 108

### N-(2-(5-fluoro-2-(4-methoxyphenylamino)pyrimidin-4-ylamino)phenyl)-N-methylmethanesulfonamide

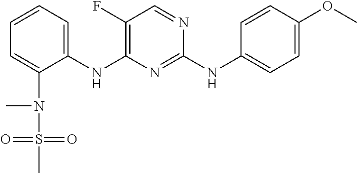

Synthesized according to the procedure in Example 1 using Intermediate 1e and the appropriate aniline derivative. ¹H NMR (d₆-DMSO) δ 7.52 (d, 1H), 7.13 (d, 1H), 6.75 (d, 1H), 6.55-6.01 (m, 3H), 6.45 (t, 1H), 6.03 (d, 2H), 2.99 (s, 3H), 2.46 (s, 3H), 2.23 (s, 3H); LCMS method A, (ES+) 418, RT=2.28 min.

### Example 109

### N-(2-(2-(3,4-dimethoxyphenylamino)-5-fluoropyrimidin-4-ylamino)phenyl)-N-methylmethanesulfonamide

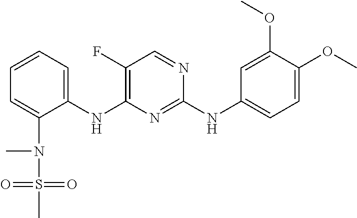

Synthesized according to the procedure in Example 1 using Intermediate 1e and the appropriate aniline derivative. ¹H NMR (d₆-DMSO) δ 7.48 (d, 1H), 7.16 (d, 1H), 6.76 (d, 1H), 6.58 (t, 1H), 6.42-6.46 (m, 2H), 6.21 (d, 1H), 6.05 (d, 1H), 3.01 (s, 3H), 2.87 (s, 3H), 2.46 (s, 3H), 2.23 (s, 3H); LCMS method A, (ES+) 448, RT=2.29 min.

### Example 110

### N-(2-(5-fluoro-2-(4-(2-(piperidin-1-yl)ethoxy)phenylamino)pyrimidin-4-ylamino)phenyl)-N-methylmethanesulfonamide

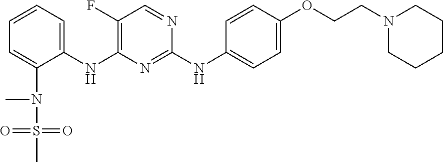

Synthesized according to the procedure in Example 1 using Intermediate 1e and the appropriate aniline derivative. ¹H NMR (d₆-DMSO) δ 7.72 (s, 1H), 7.53 (d, 1H), 7.15 (d, 1H), 6.77 (d, 1H), 6.68 (d, 2H), 6.59 (t, 1H), 6.45 (t, 1H), 6.12 (d, 2H), 3.50 (t, 2H), 2.60 (t, 2H), 2.46 (s, 3H), 2.34-2.39 (m, 4H), 2.24 (s, 3H), 1.03-1.07 (m, 4H), 0.88 (t, 2H); LCMS method A, (ES+) 515, RT=2.48 min.

### Example 111

### N-(2-(5-fluoro-2-(4-(2-morpholinoethoxy)phenylamino)pyrimidin-4-ylamino)phenyl)-N-methylmethanesulfonamide

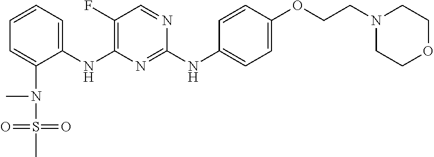

Synthesized according to the procedure in Example 1 using Intermediate 1e and the appropriate aniline derivative. ¹H NMR (d₆-DMSO) δ 7.52 (d, 1H), 7.40 (s, 2H), 7.15 (d, 1H), 6.77 (d, 1H), 6.65 (d, 2H), 6.59 (t, 1H), 6.45 (t, 1H), 6.11 (d, 2H), 3.48 (t, 2H), 3.07 (t, 4H), 2.49 (t, 2H), 2.46 (s, 3H), 2.30 (t, 4H), 2.23 (s, 3H); LCMS method A, (ES+) 517, RT=2.48 min.

### Example 112

### N-(2-(5-fluoro-2-(3-(2-(piperidin-1-yl)ethoxy)phenylamino)pyrimidin-4-ylamino)-6-methylphenyl)methanesulfonamide

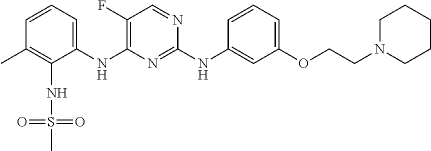

Synthesized according to the procedure in Example 1 using Intermediate 1g and the appropriate aniline derivative. ¹H NMR (d₆-DMSO) δ 9.21 (s, 1H), 8.79 (br s, 1H), 8.30 (s, 1H), 8.16 (d, 1H), 7.77 (d, 1H), 7.31 (s, 1H), 7.28 (t, 1H), 7.16-7.11 (m, 2H), 7.04 (t, 1H), 6.44 (dd, 1H), 3.88 (t, 2H), 2.92 (s, 3H), 2.60 (t, 2H), 2.43-2.37 (m, 7H), 1.52-1.46 (m, 4H), 1.38-1.35 (m, 2H); LCMS method A, (ES+) 515, RT=1.72 min.

### Example 113

### N-(2-(2-(3,5-dimethoxy-4-(2-(piperidin-1-yl)ethoxy)phenylamino)-5-fluoropyrimidin-4-ylamino)-6-methylphenyl)methanesulfonamide

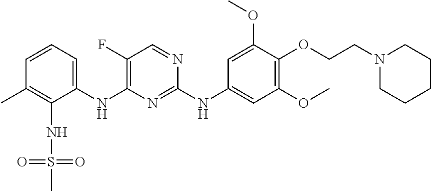

Synthesized according to the procedure in Example 1 using Intermediate 1g and the appropriate aniline derivative. ¹H NMR (d₆-DMSO) δ 9.11 (s, 1H), 8.88 (br s, 1H), 8.75 (s, 1H), 8.16 (d, 1H), 7.69 (d, 1H), 7.22 (t, 1H), 7.12 (d, 1H), 6.98 (s, 2H), 3.83 (t, 2H), 3.52 (s, 6H), 2.93 (s, 3H), 2.56 (t, 2H), 2.42-2.39 (m, 7H), 1.51-1.46 (m, 4H), 1.38-1.36 (m, 2H); LCMS method C, (ES+) 575, RT=2.40 min.

### Example 114

### N-(2-(2-(3,4-dimethoxy-5-(2-(piperidin-1-yl)ethoxy)phenylamino)-5-fluoropyrimidin-4-ylamino)-6-methylphenyl)methanesulfonamide

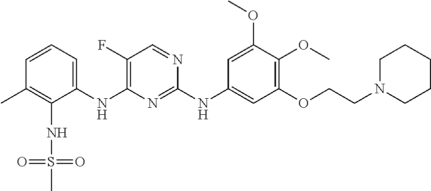

Synthesized according to the procedure in Example 1 using Intermediate 1g and the appropriate aniline derivative. ¹H NMR (d₆-DMSO) δ 9.15 (s, 1H), 8.80 (s, 1H), 8.22 (d, 1H), 7.76 (d, 1H), 7.29 (t, 1H), 7.18 (d, 1H), 7.04 (s, 2H), 3.84 (t, 2H), 3.65 (s, 3H), 3.58 (s, 3H), 2.99 (s, 3H), 2.49 (m, 4H), 2.46 (s, 3H), 1.57-1.52 (m, 4H), 1.43 (m, 2H); LCMS method A, (ES+) 575, RT=1.73 min.

### Example 115

### N-(2-(5-fluoro-2-(3-(2-methoxyethoxy)phenylamino)pyrimidin-4-ylamino)-6-methylphenyl)methanesulfonamide

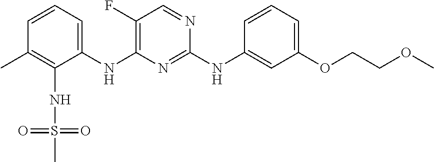

Synthesized according to the procedure in Example 1 using Intermediate 1g and the appropriate aniline derivative. ¹H NMR (d₆-DMSO) δ 9.97 (br s, 2H), 8.79 (s, 1H), 8.25 (d, 1H), 7.52 (d, 1H), 7.33-7.26 (m, 4H), 6.78 (d, 2H), 4.03 (t, 2H), 3.63 (t, 2H), 3.30 (s, 3H), 2.96 (s, 3H), 2.39 (s. 3H); LCMS method B, (ES+) 462, RT=7.20 min.

### Example 116

### N-(2-(5-fluoro-2-(3,4,5-trimethoxyphenylamino)pyrimidin-4-ylamino)-6-methylphenyl)methanesulfonamide

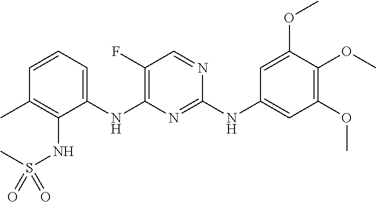

Synthesized according to the procedure in Example 1 using Intermediate 1g and the appropriate aniline derivative. ¹H NMR: (d₆-DMSO) δ 9.12 (s, 1H), 8.86 (s, 1H), 8.73 (s, 1H), 8.17 (d, 1H), 7.70 (d, 1H), 7.23 (t, 1H), 7.13 (d, 1H), 6.99 (s, 2H), 3.57 (s, 3H), 3.52 (s, 6H), 2.93 (s, 3H), 2.39 (s, 3H); LCMS method A, (ES+) 478, RT=2.17 min.

### Example 117

### N-(2-(2-(3-ethoxy-4,5-dimethoxyphenylamino)-5-fluoropyrimidin-4-ylamino)-6-methylphenyl)methanesulfonamide

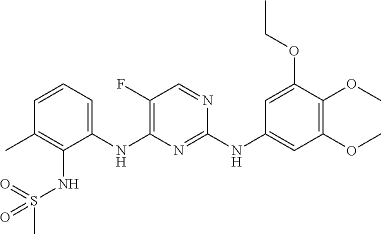

Synthesized according to the procedure in Example 1 using Intermediate 1g and the appropriate aniline derivative. ¹H NMR (d₆-DMSO) δ 9.11 (s, 1H), 8.81 (br s, 1H), 8.76 (br s, 1H), 8.16 (d, 1H), 7.64 (d, 1H), 7.21 (t, 1H), 7.13 (d, 1H), 7.01 (s, 1H), 6.89 (s, 1H), 3.55-3.65 (m, 5H), 3.53 (s, 3H), 2.90 (s, 3H), 2.38 (s, 3H), 1.22 (t, 3H); LCMS method B, (ES+) 492, RT=8.31 min.

### Example 118

### N-(2-(5-fluoro-2-(3-isopropoxy-4,5-dimethoxyphenylamino)pyrimidin-4-ylamino)-6-methylphenyl)methanesulfonamide

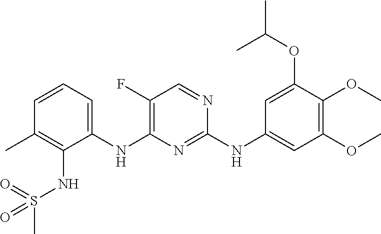

Synthesized according to the procedure in Example 1 using Intermediate 1g and the appropriate aniline derivative. ¹H NMR (d₆-DMSO) δ 9.08 (s, 1H), 8.86 (s, 1H), 8.30 (s, 1H), 8.16 (d, 1H), 7.68 (d, 1H), 7.23 (t, 1H), 7.14 (d, 1H), 6.97 (d, 2H), 4.13 (m, 1H), 3.56 (s, 3H), 3.51 (s, 3H), 2.91 (s, 3H), 2.39 (s, 3H), 1.16 (d, 6H); LCMS method A, (ES+) 506, RT=2.45 min.

### Example 119

### N-(2-(5-fluoro-2-(3-isobutoxy-4,5-dimethoxyphenylamino)pyrimidin-4-ylamino)-6-methylphenyl)methanesulfonamide

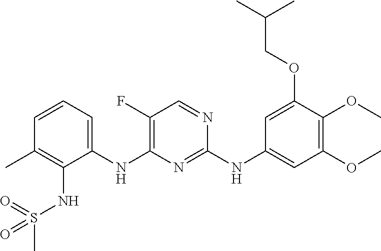

Synthesized according to the procedure in Example 1 using Intermediate 1g and the appropriate aniline derivative. ¹H NMR (d₆-DMSO) δ 9.08 (s, 1H), 8.87 (s, 1H), 8.73 (s, 1H), 8.16 (d, 1H), 7.71 (d, 1H), 7.21 (t, 1H), 7.12 (d, 1H), 6.96 (d, 2H), 3.59 (s, 3H), 3.45-3.55 (m, 5H), 2.92 (s, 3H), 2.38 (s, 3H), 1.85-2.00 (m, 1H), 0.94 (d, 6H); LCMS method B, (ES+) 520, RT=9.84 min.

### Example 120

### N-(2-(2-(3-(cyclopropylmethoxy)-4,5-dimethoxyphenylamino)-5-fluoropyrimidin-4-ylamino)-6-methylphenyl)methanesulfonamide

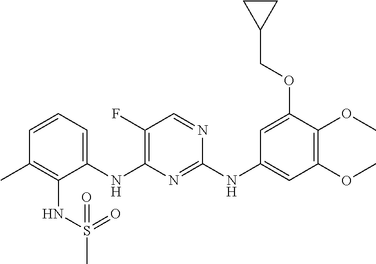

Synthesized according to the procedure in Example 1 using Intermediate 1g and the appropriate aniline derivative. ¹H NMR (d₆-DMSO) δ 9.08 (s, 1H), 8.77 (s, 1H), 8.15 (d, 1H), 7.70 (d, 1H), 7.20 (t, 1H), 7.11 (d, 1H), 6.95 (d, 2H), 3.45-3.65 (m, 8H), 2.90 (s, 3H), 2.38 (s, 3H), 1.10-1.2 (m, 1H), 0.47-0.55 (m, 2H), 0.20-0.30 (m, 2H); LCMS method B, (ES+) 518, RT=9.10 min.

### Example 121

### N-(2-(5-fluoro-2-(3-isopropoxyphenylamino)pyrimidin-4-ylamino)-6-methylphenyl)methanesulfonamide

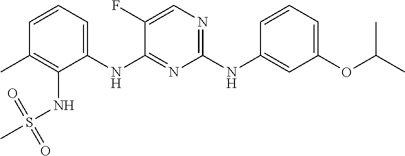

Synthesized according to the procedure in Example 1 using Intermediate 1g and the appropriate aniline derivative. ¹H NMR (d₆-DMSO) δ 9.21 (s, 1H), 8.90 (s, 1H), 8.74 (s, 1H), 8.17 (d, 1H), 7.77 (d, 1H), 7.30-7.26 (m, 2H), 7.16-7.11 (m, 2H), 7.05 (t, 1H), 6.41 (d, 1H), 4.35 (m, 1H), 2.94 (s, 3H), 2.40 (s, 3H), 1.20 (d, 6H); LCMS method A, (ES+) 446 RT=2.62 min.

### Example 122

### N-(2-(5-fluoro-2-(3-propoxyphenylamino)pyrimidin-4-ylamino)-6-methylphenyl)methanesulfonamide

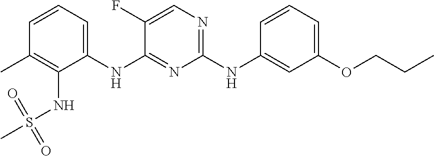

Synthesized according to the procedure in Example 1 using Intermediate 1g and the appropriate aniline derivative. ¹H NMR (d₆-DMSO) δ 9.23 (s, 1H), 8.89 (s, 1H), 8.74 (s, 1H), 8.18 (d, 1H), 7.75 (d, 1H), 7.32 (d, 1H), 7.27 (t, 1H), 7.15-7.11 (m, 2H), 7.04 (td, 1H), 6.43 (dd, 1H), 3.70 (t, 2H), 2.94 (s, 3H), 2.40 (s, 3H), 1.67 (m, 2H), 0.95 (t, 3H); LCMS method A, (ES+) 446 RT=2.68 min.

### Example 123

### N-(2-(5-chloro-2-(3-(2-hydroxyethoxy)phenylamino)pyrimidin-4-ylamino)phenyl)methanesulfonamide

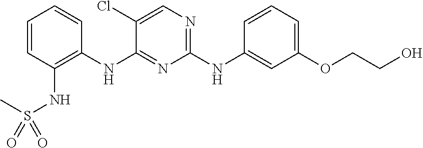

Synthesized according to the procedure in Example 1 using Intermediate 1h and the appropriate aniline derivative. ¹H NMR: (d₆-DMSO) δ 10.23 (br s, 1H), 9.47 (br s, 1H), 9.41 (s, 1H), 8.43 (s, 1H), 7.82 (s, 1H), 7.56 (m, 1H), 7.43 (m, 2H), 7.11 (m, 3H), 6.67 (s, 1H), 3.90 (t, 2H), 3.78 (t, 2H) 3.03 (s, 3H); LCMS method A, (ES+) 450, RT=2.17 min.

### Example 124

### N-(2-(5-chloro-2-(3-(2-(piperidin-1-yl)ethoxy)phenylamino)pyrimidin-4-ylamino)phenyl)methanesulfonamide

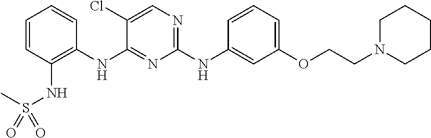

Synthesized according to the procedure in Example 1 using Intermediate 1h and the appropriate aniline derivative. ¹H NMR: (d₆-DMSO) δ 10.70 (s, 1H), 10.27 (s, 1H), 9.42 (s, 1H), 9.39 (s, 1H), 8.36 (s, 1H), 7.73 (d, 1H), 7.49 (d, 1H), 7.36 (m, 2H), 7.06 (d, 2H), 6.63 (s, 1H), 4.27 (t, 2H), 3.47-3.35 (m, 4H), 3.00-2.90 (m, 5H), 1.85-1.60 (m, 5H), 1.37 (m, 1H); LCMS method A, (ES+) 517, RT=1.82 min.

### Example 125

### N-(2-(5-chloro-2-(4-methoxy-3-(2-(piperidin-1-yl)ethoxy)phenylamino)pyrimidin-4-ylamino)phenyl)methanesulfonamide

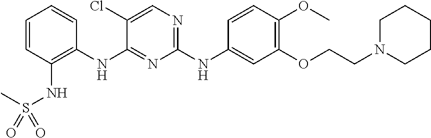

Synthesized according to the procedure in Example 1 using Intermediate 1h and the appropriate aniline derivative. ¹H NMR: (d₆-DMSO) δ 9.32 (br s, 1H), 9.13 (s, 1H), 8.60 (s, 1H), 8.15 (s, 1H), 8.05 (d, 1H), 7.39 (d, 1H), 7.30-7.09 (m, 4H), 6.78 (d, 1H), 3.88 (t, 2H), 3.70 (s, 3H), 2.93 (s, 3H), 2.65 (t, 2H), 2.45 (br s), 1.49 (t, 4H), 1.38 (br m, 2H); LCMS method A, (ES+) 547, RT=1.79 min.

### Example 126

### N-(2-(5-chloro-2-(3-(2-(diethylamino)ethoxy)phenylamino)pyrimidin-4-ylamino)phenyl)methanesulfonamide

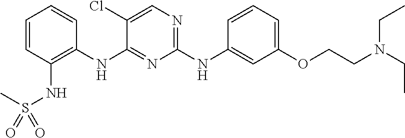

Synthesized according to the procedure in Example 1 using Intermediate 1h and the appropriate aniline derivative. ¹H NMR: (d₆-DMSO) δ 10.69 (br s, 1H), 10.34 (br s, 1H), 9.49 (br s, 1H), 9.41 (s, 1H), 8.37 (s, 1H), 7.72 (d, 1H), 7.50 (d, 1H), 7.37 (m, 2H), 7.06 (m, 3H), 6.64 (m, 1H), 4.24 (t, 2H), 3.43 (t, 2H), 3.17 (m, 4H), 2.94 (s, 3H), 1.24 (t, 6H); LCMS method A, (ES+) 505, RT=1.83 min.

### Example 127

### N-(2-(5-chloro-2-(4-(2-(diethylamino)ethoxy)phenylamino)pyrimidin-4-ylamino)phenyl)methanesulfonamide

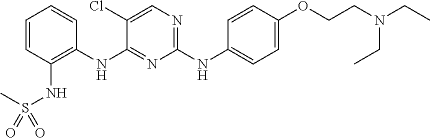

Synthesized according to the procedure in Example 1 using Intermediate 1h and the appropriate aniline derivative. ¹H NMR: (d₆-DMSO) δ 10.53 (br s, 1H), 10.23 (br s, 1H), 9.51 (br s, 1H), 9.36 (s, 1H), 8.33 (s, 1H), 7.69 (br s, 1H), 7.50 (d, 1H), 7.43-7.29 (m, 4H), 6.80 (d, 2H), 4.32 (t, 2H), 3.46 (t, 2H), 3.19 (m, 4H), 2.93 (s, 3H), 1.26 (t, 6H); LCMS method A, (ES+) 505, RT=1.67 min.

### Example 128

### N-(2-(5-chloro-2-(3-(2-(piperidin-1-yl)ethoxy)phenylamino)pyrimidin-4-ylamino)-6-methylphenyl)methanesulfonamide

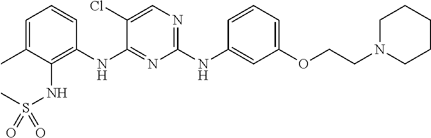

Synthesized according to the procedure in Example 1 using Intermediate 1h and the appropriate aniline derivative. ¹H NMR: (d₆-DMSO) δ 8.55 (s, 1H), 8.01 (s, 1H), 7.80 (d, 1H), 7.34 (t, 1H), 7.23 (t, 1H), 7.20 (d, 1H), 7.12 (t, 1H), 7.04 (d, 1H), 6.59 (d, 1H), 4.04 (t, 2H), 3.37 (s, 3H), 3.20 (t, 2H), 3.05-2.90 (br s, 4H), 3.02 (s, 3H), 2.49 (s, 3H), 1.80 (m, 4H), 1.63 (m, 2H); LCMS method A, (ES+) 531, RT=1.86 min.

### Example 129

### N-(2-(5-chloro-2-(3-(piperidin-4-ylmethoxy)phenylamino)pyrimidin-4-ylamino)phenyl)methanesulfonamide

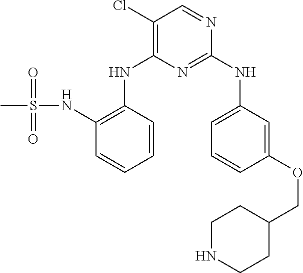

N-(2-(5-chloro-2-(3-(N-Boc-piperidin-4-ylmethoxy)phenylamino)pyrimidin-4-ylamino)phenyl)methanesulfonamide, prepared according to the procedure in Example 1 using Intermediate 1h, was treated with 50% TFA in DCM at room temperature then concentrated in vacuo and purified by HPLC. ¹H NMR (d₆-DMSO) δ 9.32 (br s, 1H), 9.28 (br s, 1H), 8.32 (d, 1H), 8.14 (s, 1H), 7.40 (s, 1H), 7.22 (dd, 2H), 7.13 (t, 1H), 6.88 (dd, 1H), 6.71 (dd, 1H), 6.52 (d, 1H), 3.78 (d, 2H), 3.21 (d, 2H), 2.78 (t, 2H), 2.71 (s, 3H), 1.97-1.93 (m, 1H), 1.83 (d, 2H), 1.35 (quartet, 2H); LCMS method A, (ES+) 503, RT=1.80 min.

### Example 130

### N-(2-(5-chloro-2-(3-(2-(2-oxopyrrolidin-1-yl)ethoxy)phenylamino)pyrimidin-4-ylamino)phenyl)methanesulfonamide

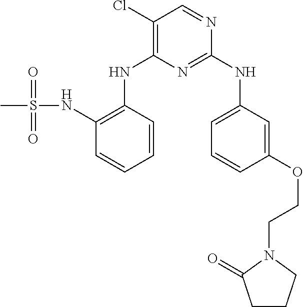

Synthesized according to the procedure in Example 1 using Intermediate 1h and the appropriate aniline derivative. ¹H NMR (d₆-DMSO) δ 9.35 (br s, 1H), 8.56 (s, 1H), 8.21 (s, 1H), 7.96 (d, 1H), 7.42 (d, 1H), 7.35 (dd, 1H), 7.26 (dd, 1H), 7.21 (br s, 1H), 7.19 (d, 1H), 7.04 (dd, 1H), 6.49 (d, 1H), 3.92 (t, 2H), 3.51 (t, 2H), 3.42 (t, 2H), 2.96 (s, 3H), 2.22 (t, 2H), 1.94-1.87 (m, 2H); LCMS method C, (ES+) 517, RT=2.03 min.

### Example 131

### N-(2-(5-chloro-2-(3-(2-(pyrrolidin-2-yl)ethoxy)phenylamino)pyrimidin-4-ylamino)phenyl)methanesulfonamide

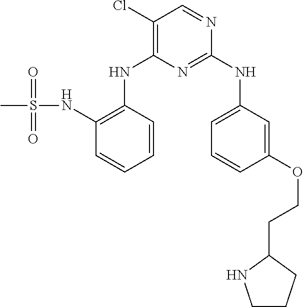

N-(2-(5-chloro-2-(3-(2-(N-Boc-pyrrolidin-2-yl)ethoxy)phenylamino)pyrimidin-4-ylamino)phenyl)methanesulfonamide, prepared according to the procedure in Example 1 using Intermediate 1h, was treated with 50% TFA in DCM at room temperature then concentrated in vacuo and purified by HPLC. ¹H NMR (d₆-DMSO) δ 9.38 (s, 1H), 8.89 (s, 1H), 8.33 (s, 1H), 8.18 (s, 1H), 8.10 (d, 1H), 7.35 (d, 1H), 7.33 (d, 1H), 7.18 (d, 1H), 7.13-7.07 (m, 3H), 6.51 (d, 1H), 3.97-3.88 (m, 2H), 3.55-3.46 (m, 1H), 3.21-3.06 (m 2H), 2.85 (s, 3H), 2.17-2.06 (m, 2H), 2.05-1.99 (m, 1H), 1.94-1.78 (m, 2H), 1.64-1.51 (m, 1H); LCMS method B, (ES+) 503, RT=5.70 min.

### Example 132

### N-(4-(5-chloro-4-(2-(methylsulfonamido)phenylamino)pyrimidin-2-ylamino)phenyl)-2-(pyrrolidin-1-yl)acetamide

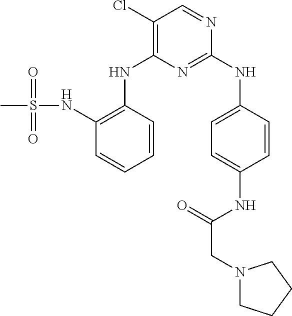

Synthesized according to the procedure in Example 1 using Intermediate 1h and the appropriate aniline derivative. ¹H NMR (d₆-DMSO) δ 8.38 (s, 1H), 8.04 (s, 1H), 8.01 (d, 1H), 7.46-7.35 (m, 6H), 7.27 (dd, 1H), 4.17 (s, 2H), 3.33 (s, 2H), 2.12 (s, 2H); LCMS method B, (ES+) 516, RT=5.23 min.

### Example 133

### N-(2-(5-chloro-2-(3-(2-(piperazin-1-yl)ethoxy)phenylamino)pyrimidin-4-ylamino)phenyl)methanesulfonamide trifluoroacetate

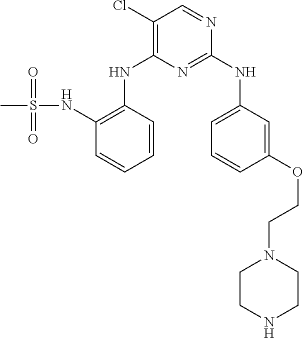

N-(2-(5-chloro-2-(3-(2-(N-Boc-piperazin-1-yl)ethoxy)phenylamino)pyrimidin-4-ylamino)phenyl)methanesulfonamide trifluoroacetate, prepared according to the procedure in Example 1 using Intermediate 1h, was treated with 50% TFA in DCM at room temperature then concentrated in vacuo. ¹H NMR (d₆-DMSO) δ 9.46 (s, 1H), 9.33 (s, 1H), 8.97 (br s, 1H), 8.64 (s, 1H), 8.22 (s, 1H), 7.94 (d, 1H), 7.43 (d, 1H), 7.35 (dd, 1H), 7.28 (d, 1H), 7.25 (s, 1H), 7.20 (s, 1H), 7.08 (dd, 1H), 6.54 (d, 1H), 4.10 (t, 2H), 3.32 (t, 2H), 2 extra peaks not visible under water peak; LCMS method B, (ES+) 518, RT=5.17 min.

### Example 134

### N-(2-(5-Chloro-2-(3-(3-piperidin-1-yl)propoxy)phenylamino)pyrimidin-4-ylamino)phenyl)methanesulfonamide

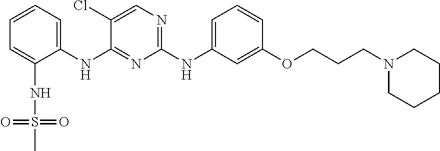

Synthesized according to the procedure in Example 1 using Intermediate 1h and the appropriate aniline derivative. ¹H NMR (d₆-DMSO) δ 9.34 (s, 1H), 8.66 (s, 1H), 8.19 (s, 1H), 8.02 (d, 1H), 7.39 (dd, 1H), 7.14-7.26 (m, 4H), 7.05 (t, 1H), 6.47 (dd, 1H), 3.84 (t, 2H), 2.92 (s, 3H), 2.44-2.45 (m, 6H), 1.86 (t, 2H), 1.51-1.52 (m, 4H), 1.40-1.41 (m, 2H); LCMS method A, (ES+)=533, 531, RT=1.88 min.

### Example 135

### N-(2-(5-chloro-2-(3-(3-methylpiperazin-1-yl)propoxy)phenylamino)pyrimidin-4-ylamino)phenyl)methanesulfonamide

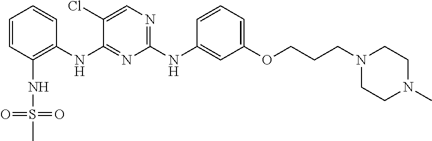

Synthesized according to the procedure in Example 1 using Intermediate 1h and the appropriate aniline derivative. ¹H NMR (d₆-DMSO) δ 8.36 (s, 3H), 8.10 (s, 1H), 8.05 (d, 1H), 7.46 (d, 1H), 7.35-7.39 (m, 1H), 7.23-7.30 (m, 2H), 7.10 (t, 1H), 6.99-7.01 (m, 1H), 6.53-6.55 (m, 1H), 3.92 (t, 2H), 3.32-3.33 (m, 4H), 3.10 (s, 3H), 2.97 (m, 4H), 2.87-2.88 (m, 2H), 2.67 (s, 3H), 2.00-2.01 (m, 2H); LCMS method A, (ES+)=546, RT=1.69 min.

### Example 136

### N-(2-(5-chloro-2-(4-(3-(4-methylpiperazin-1-yl)propoxy)phenylamino)pyrimidin-4-ylamino)phenyl)methanesulfonamide

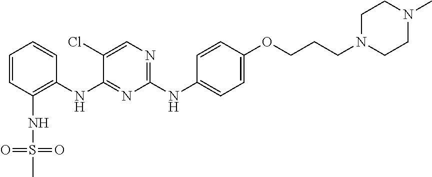

Synthesized according to the procedure in Example 1 using Intermediate 1h and the appropriate aniline derivative. ¹H NMR (d₆-DMSO) δ 9.19 (s, 1H), 8.57 (s, 1H), 8.13 (s, 1H), 7.99 (d, 1H), 7.38-7.44 (m, 3H), 7.22-7.30 (m, 2H), 6.74 (d, 2H), 3.93 (t, 2H), 2.92 (s, 3H), 2.37-2.43 (m, 10H), 2.18 (s, 3H), 1.82-1.85 (m, 2H); LCMS method A, (ES+) 546, RT=1.56 min.

### Example 137

### N-(2-(5-chloro-2-(3-isobutoxy-4,5-dimethoxyphenylamino)pyrimidin-4-ylamino)phenyl)methanesulfonamide

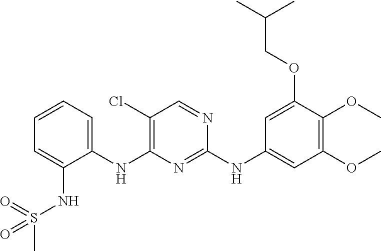

Synthesized according to the procedure in Example 1 using Intermediate 1h and the appropriate aniline derivative. ¹H NMR (d₆-DMSO) δ 9.33 (s, 1H), 9.21 (s, 1H), 8.53 (s, 1H), 8.20 (s, 1H), 7.99 (d, 1H), 7.39 (d, 1H), 7.15-7.35 (m, 2H), 6.93 (d, 2H), 3.60 (s, 3H), 3.45-3.55 (m, 5H), 2.96 (s, 3H), 1.90-2.05 (m, 1H), 0.95 (d, 6H); LCMS method B, (ES+) 522, RT=10.56 min.

### Example 138

### N-(2-(5-chloro-2-(3,4,5-trimethoxyphenylamino)pyrimidin-4-ylamino)phenyl)methanesulfonamide hydrochloride

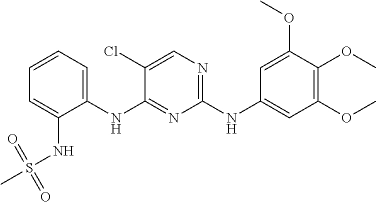

Synthesized according to the procedure in Example 1 using Intermediate 1h and the appropriate aniline derivative. ¹H NMR (d₆-DMSO) δ 9.95 (br s, 1H), 9.36 (s, 1H), 9.24 (br s, 1H), 8.30 (s, 1H), 7.75 (d, 1H), 7.44 (d, 1H), 7.30-7.25 (m, 2H), 6.78 (s, 2H), 3.59 (s, 3H), 3.57 (s, 6H), 2.96 (s, 3H); LCMS method A, (ES+) 480, 482, RT=2.33 min.

### Example 139

### N-(2-(5-chloro-2-(4-methoxyphenylamino)pyrimidin-4-ylamino)phenyl)methanesulfonamide hydrochloride

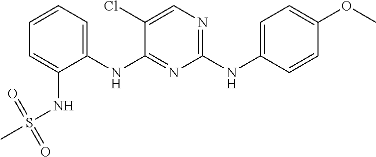

Synthesized according to the procedure in Example 1 using Intermediate 1h and the appropriate aniline derivative. ¹H NMR (d₆-DMSO) δ 10.15 (br s, 1H), 9.52 (br s, 1H), 9.32 (s, 1H), 8.31 (s, 1H), 7.67 (d, 1H), 7.49 (dd, 1H), 7.37-7.32 (m, 2H), 7.26 (d, 2H), 6.75 (d, 2H), 3.71 (s, 3H), 2.93 (s, 3H); LCMS method A, (ES+) 420, 422, RT=2.26 min.

### Example 140

### N-(2-(5-chloro-2-(3-(2-(pyrrolidin-1-yl)ethoxy)phenylamino)pyrimidin-4-ylamino)phenyl)methanesulfonamide

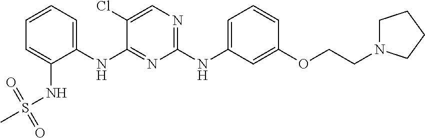

Synthesized according to the procedure in Example 1 using Intermediate 1h and the appropriate aniline derivative. ¹H NMR (d₆-DMSO) δ 9.38 (br s, 1H), 9.33 (s, 1H), 8.65 (s, 1H), 8.19 (s, 1H), 8.03 (d, 1H), 7.39 (dd, 1H), 7.27 (m, 2H), 7.21 (dd, 2H), 7.05 (t, 1H), 6.49 (dd, 1H), 3.94 (t, 2H), 2.93 (s, 3H), 2.82 (t, 2H), 2.57 (m, 4H), 1.71 (m, 4H); LCMS method C, (ES+) 503, 505 RT=2.37 min.

### Example 141

### N-(2-(5-chloro-2-(3-methoxy-4-(2-(pyrrolidin-1-yl)ethoxy)phenylamino)pyrimidin-4-ylamino)phenyl)methanesulfonamide

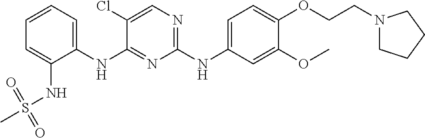

Synthesized according to the procedure in Example 1 using Intermediate 1h and the appropriate aniline derivative. ¹H NMR (d₆-DMSO) δ 9.18 (s, 1H), 8.61 (s, 1H), 8.19 (s, 1H), 8.15 (s, 1H), 8.02 (d, 1H), 7.38 (dd, 1H), 7.25-7.18 (m, 3H), 7.11 (d, 1H), 6.79 (d, 1H), 4.00 (t, 2H), 3.57 (s, 3H), 2.92 (s, 3H), 2.87 (t, 2H), 2.64 (m, 4H), 1.73 (m, 4H); LCMS method C, (ES+) 533, 535 RT=1.92 min.

### Example 142

### N-(2-(5-chloro-2-(3-methoxy-4-(2-(piperidin-1-yl)ethoxy)phenylamino)pyrimidin-4-ylamino)phenyl)methanesulfonamide hydrochloride

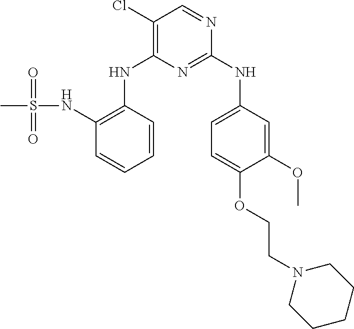

Synthesized according to the procedure in Example 1 using Intermediate 1h and the appropriate aniline derivative. ¹H NMR (d₆-DMSO) δ 10.54 (br s, 1H), 9.99 (br s, 1H), 9.38 (s, 1H), 9.25 (br s, 1H), 7.77 (br s, 1H), 7.45-7.47 (m, 1H), 7.30-7.32 (m, 2H), 7.11 (s, 1H), 6.98 (d, 1H), 6.86 (d, 1H), 4.32 (t, 1H), 3.54 (s, 3H), 3.49-3.54 (m, 2H), 3.41 (quartet, 2H), 2.98-3.04 (m, 2H), 2.94 (s, 3H), 1.76-1.84 (m, 3H), 1.68-1.74 (m, 1H), 1.35-1.44 (m, 1H); LCMS method B, (ES+) 547, 549, RT=5.38 min.

### Example 143

### Isopropyl 2-(4-(5-chloro-4-(2-(methylsulfonamido)phenylamino)pyrimidin-2-ylamino)-phenoxy)acetate hydrochloride

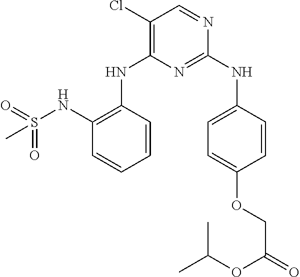

Synthesized according to the procedure in Example 1 using Intermediate 1h and the appropriate aniline derivative. ¹H NMR (d₆-DMSO) δ 9.74 (br s, 1H), 9.33 (s, 1H), 9.01 (br s, 1H), 8.27 (s, 1H), 7.85 (d, 1H), 7.44 (d, 1H), 7.34-7.38 (m, 1H), 7.27-7.32 (m, 1H), 7.11 (d, 2H), 7.04 (t, 1H), 6.49 (dd, 1H), 4.96-5.02 (m, 1H), 4.61 (s, 2H), 2.95 (s, 3H), 1.22 (d, 6H); LCMS method B, (ES+) 506, 508, RT=10.20 min.

### Example 144

### Ethyl 2-(4-(5-fluoro-4-(2-(methylsulfonamido)phenylamino)pyrimidin-2-ylamino)phenoxy)acetate hydrochloride

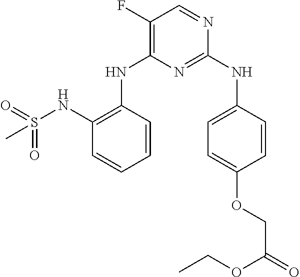

Synthesized according to the procedure in Example 1 using Intermediate 1h and the appropriate aniline derivative. ¹H NMR (d₆-DMSO) δ 9.67 (br s, 1H), 9.29 (s, 1H), 8.22 (d, 1H), 7.34 (d, 1H), 7.47 (dd, 1H), 7.26-7.38 (m, 2H), 7.10-7.13 (m, 1H), 7.04 (t, 1H), 6.49 (d, 1H), 4.63 (s, 1H), 4.14-4.20 (m, 1H), 2.94 (s, 3H), 1.22 (t, 3H); LCMS method B, (ES+) 476, RT=8.75 min.

### Example 145

### N-(2-(5-chloro-2-(3-((1-methylpiperidin-2-yl)methoxy)phenylamino)pyrimidin-4-ylamino)phenyl)methanesulfonamide

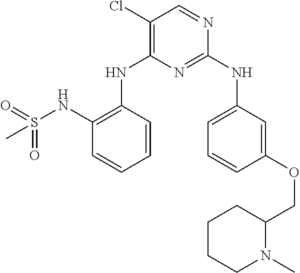

Synthesized according to the procedure in Example 1 using Intermediate 1h and the appropriate aniline derivative. ¹H NMR (d₆-DMSO) δ 9.35 (s, 1H), 8.70 (s, 1H), 8.20 (s, 2H), 8.03 (d, 1H), 7.38 (dd, 1H), 7.21-7.74 (m, 1H), 7.14-7.21 (m, 2H), 7.04 (t, 1H), 6.47 (dd, 1H), 3.65-3.76 (m, 2H), 2.91 (s, 3H), 2.86 (d, 1H), 2.66-2.73 (m, 1H), 2.23 (s, 3H), 1.96-2.03 (m, 2H), 1.82-1.88 (m, 1H), 1.63-1.73 (m, 2H), 1.45-1.56 (m, 1H), 0.97-1.08 (m, 1H); LCMS method B, (ES+) 517, 519, RT=5.82 min.

### Example 146

### N-(2-(5-chloro-2-(3-((1-methylpiperidin-3-yl)methoxy)phenylamino)pyrimidin-4-ylamino)phenyl)methanesulfonamide

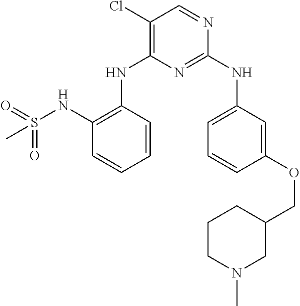

Synthesized according to the procedure in Example 1 using Intermediate 1h and the appropriate aniline derivative. ¹H NMR (d₆-DMSO) δ 9.35 (s, 1H), 8.64 (s, 1H), 8.20 (s, 1H), 8.18 (s, 1H), 8.00 (d, 1H), 7.40 (dd, 1H), 7.28 (t, 1H), 7.22-7.24 (m, 2H), 7.15 (d, 1H), 7.04 (t, 1H), 6.47 (dd, 1H), 3.68-3.77 (m, 2H), 2.93 (s, 3H), 2.90-2.92 (m, 1H), 2.75-2.82 (m, 1H), 2.30 (s, 3H), 1.90-2.12 (m, 3H), 1.60-1.72 (m, 2H), 1.49-1.51 (m, 1H), 0.97-1.10 (m, 1H); LCMS method B, (ES+) 517, 519, RT=5.84 min.

### Example 147

### N-(2-(5-chloro-2-(3-((1,4-dimethylpiperazin-2-yl)methoxy)phenylamino)pyrimidin-4-ylamino)phenyl)methanesulfonamide

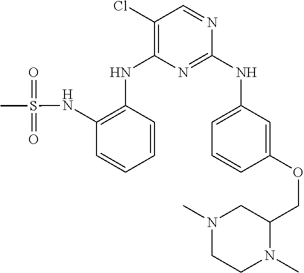

Synthesized according to the procedure in Example 1 using Intermediate 1h and the appropriate aniline derivative. ¹H NMR (d₆-DMSO) δ 9.36 (s, 1H), 8.61 (s, 1H), 8.21 (s, 1H), 8.00 (d, 1H), 7.41 (d, 1H), 7.32 (dd, 1H), 7.24 (dd, 1H), 7.17 (d, 1H), 6.50 (d, 1H), 4.01 (dd, 1H), 3.76 (dd, 1H), 2.94 (s, 3H), 2.77 (d, 1H), 2.72-2.68 (m, 1H), 2.60 (d, 1H), 2.44-2.40 (m, 1H), 2.24 (s, 3H), 2.19 (s, 3H), 2.12-2.07 (m, 1H), 1.94 (t, 1H), one extra proton not visible under water peak; LCMS method B, (ES+) 532, RT=5.33 min.

### Example 148

### 2-(4-(5-chloro-4-(2-(methylsulfonamido)phenylamino)pyrimidin-2-ylamino)phenyl)acetic acid

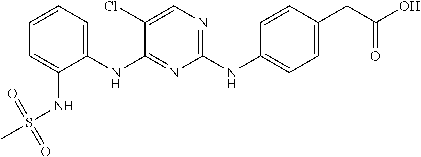

2-(4-(5-chloro-4-(2-(methylsulfonamido)phenylamino)pyrimidin-2-ylamino)phenyl)acetic acid ethyl ester, prepared according to the procedure in Example 1 using Intermediate 1h, was treated with 3M LiOH(aq) (10 eq.) in MeOH at 50° C. for 2 h, diluted with water and adjusted to pH5 with 1M hydrochloric acid. The title product was collected at the pump and dried. ¹H NMR (d₆-DMSO) δ 12.22 (br s, 1H), 9.36 (s, 1H), 9.34 (br, s, 1H), 8.56 (s, 1H), 8.18 (s, 1H), 7.97 (d, 1H), 7.47 (d, 2H), 7.42 (dd, 1H), 7.35 (td, 1H), 7.27 (td, 1H), 7.03 (d, 2H), 3.45 (s, 2H), 2.94 (s, 3H); LCMS method A, (ES+) 448, 450, RT=2.12 min.

### Example 149

### 2-(3-(5-chloro-4-(2-(methylsulfonamido)phenylamino)pyrimidin-2-ylamino)phenoxy)acetic acid hydrochloride

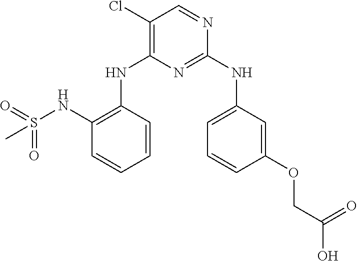

2-(3-(5-chloro-4-(2-(methylsulfonamido)phenylamino)pyrimidin-2-ylamino)phenoxy)acetic acid ethyl ester, prepared according to the procedure in Example 1 using Intermediate 1h, was dissolved in THF and treated with 1M KOH in 6:1 methanol-water at 50° C. for 3 h. The mixture was concentrated in vacuo and acidified with hydrochloric acid. The aqueous layer was extracted with DCM, the organics were dried and concentrated to afford 2-(3-(5-chloro-4-(2-(methylsulfonamido)phenylamino)pyrimidin-2-ylamino)phenoxy)acetic acid hydrochloride. ¹H NMR (d₆-DMSO) δ 9.47 (br s, 1H), 9.32 (s, 1H), 8.70 (br s, 1H), 8.22 (s, 1H), 7.95 (d, 1H), 7.42 (dd, 1H), 7.38 (dt, 1H), 7.17-7.21 (m, 2H), 7.04 (t, 1H), 6.44-6.48 (dm, 1H), 4.59 (s, 2H), 2.96 (s, 3H); LCMS method B, (ES+) 464, 466, RT=7.90 min.

### Example 150

### 2-(3-(5-chloro-4-(2-(methylsulfonamido)phenylamino)pyrimidin-2-ylamino)phenoxy)-N,N-diethylacetamide

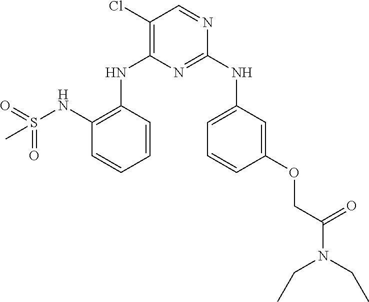

Synthesized according to the procedure in Example 1 using Intermediate 1h and the appropriate aniline derivative. ¹H NMR (d₆-DMSO) δ 9.38 (s, 1H), 8.61 (s, 1H), 8.20 (s, 1H), 8.01 (d, 1H), 7.40 (d, 1H), 7.33 (t, 1H), 7.17-7.24 (m, 3H), 7.04 (t, 1H), 6.45 (dd, 1H), 4.65 (s, 2H), 3.25-3.34 (m, 4H), 3.17 (d, 3H), 2.94 (s, 3H), 1.13 (t, 3H), 1.03 (t, 3H); LCMS method B, (ES+) 519, 521, RT=8.95 min.

### Example 151

### 2-(3-(5-chloro-4-(2-(methylsulfonamido)phenylamino)pyrimidin-2-ylamino)phenoxy)-N-ethylacetamide 2,2,2-trifluoroacetate

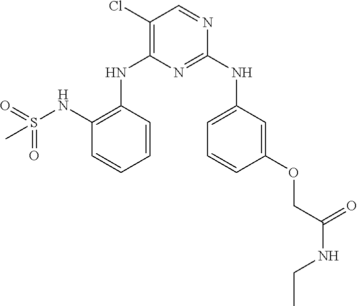

Synthesized according to the procedure in Example 1 using Intermediate 1h and the appropriate aniline derivative. ¹H NMR (d₆-DMSO) δ 9.46 (s, 1H), 9.32 (s, 1H), 8.65 (d, 1H), 8.22 (s, 1H), 8.04 (t 1H), 7.97 (d, 1H), 7.42 (d, 1H), 7.36 (t, 1H), 7.19-7.27 (m, 3H), 7.06 (t, 1H), 7.04 (d, 1H), 6.51 (dd, 1H), 4.36 (s, 1H), 3.16 (quintet, 2H), 2.96 (s, 3H), 1.04 (t, 3H); LCMS method B, (ES+) 491, 493, RT=8.37 min.

### Example 152

### N-(2-(5-chloro-2-(3,4,5-trimethoxyphenylamino)pyrimidin-4-ylamino)-5-methoxyphenyl)methanesulfonamide

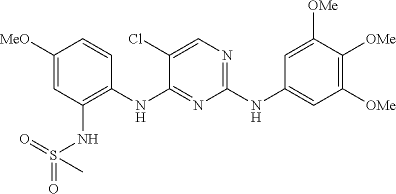

Synthesized according to the procedure in Example 1 using Intermediate 1i and the appropriate aniline derivative. ¹H NMR (d₆-DMSO) δ 9.25 (br s, 1H), 9.13 (br s, 1H), 8.38 (br s, 1H), 8.13 (s, 1H), 7.56 (d, 1H), 6.97 (d, 1H), 6.92 (s, 2H), 6.84 (d, 1H), 3.78 (s, 3H), 3.56 (s, 3H), 3.36 (s, 6H), 2.93 (s, 3H); LCMS method B, (ES+) 510, RT=8.39 min.

### Example 153

### N-(2-(5-chloro-2-(3,4-dimethoxyphenylamino)pyrimidin-4-ylamino)-5-methoxyphenyl)methanesulfonamide

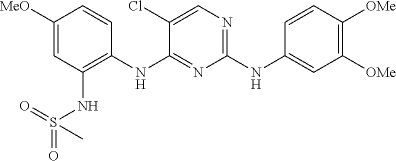

Synthesized according to the procedure in Example 1 using Intermediate 1i and the appropriate aniline derivative. ¹H NMR (d₆-DMSO) δ 9.24 (br s, 1H), 9.08 (br s, 1H), 8.36 (br s, 1H), 8.10 (s, 1H), 7.58 (d, 1H), 7.15 (s, 1H), 7.06 (d, 1H), 6.98 (m, 1H), 6.90-6.87 (m, 1H), 6.68 (d, 1H), 3.80 (s, 3H), 3.67 (s, 3H), 3.36 (s, 3H), 2.91 (s, 3H); LCMS method B, (ES+) 480, RT=7.55 min.

### Example 154

### N-ethyl-N-(2-(5-fluoro-2-(3,4,5-trimethoxyphenylamino)pyrimidin-4-ylamino)phenyl)methanesulfonamide

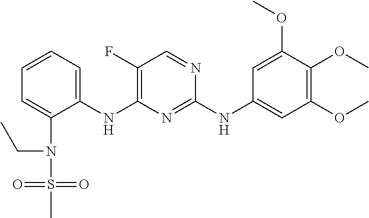

Synthesized according to the procedure in Example 1 using Intermediate 1j and the appropriate aniline derivative. ¹H NMR (d₆-DMSO) δ 9.21 (s, 1H), 8.36 (d, 1H), 8.17 (s, 1H), 8.04 (br s, 1H), 7.62 (d, 1H), 7.40 (t, 1H), 7.25 (t, 1H), 7.23 (s, 2H), 3.63 (s, 6H), 3.60 (s, 3H), 3.17 (br d, 2H), 3.12 (s, 3H), 0.95 (t, 3H); LCMS method A, (ES+) 491, RT=2.54 min.

### Example 155

### N-(2-(2-(3,5-dimethylphenylamino)-5-fluoropyrimidin-4-ylamino)phenyl)-N-ethylmethanesulfonamide

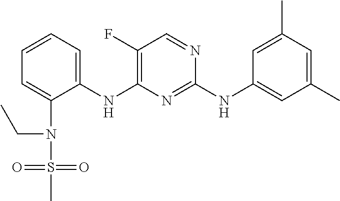

Synthesized according to the procedure in Example 1 using Intermediate 1j and the appropriate aniline derivative. ¹H NMR (d₆-DMSO) δ 9.27 (s, 1H), 8.37 (d, 1H), 8.48 (s, 1H), 8.04 (br s, 1H), 7.62 (d, 1H), 7.42 (t, 1H), 7.27 (t, 1H), 7.25 (s, 2H), 6.56 (s, 1H), 3.50 (br d, 2H), 3.12 (s, 3H), 2.18 (s, 6H), 0.93 (t, 3H); LCMS method A, (ES+) 430, RT=2.55 min.

### Example 156

### N-ethyl-N-(2-(5-fluoro-2-(4-methoxyphenylamino)pyrimidin-4-ylamino)phenyl)methanesulfonamide

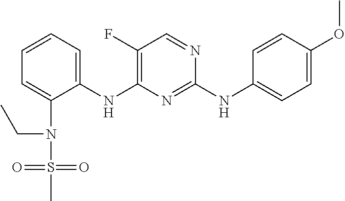

Synthesized according to the procedure in Example 1 using Intermediate 1j and the appropriate aniline derivative. ¹H NMR (d₆-DMSO) δ 9.15 (s, 1H), 8.40 (d, 1H), 8.15 (d, 1H), 7.98 (br s, 1H), 7.62 (d, 1H), 7.52 (d, 2H), 7.44 (t, 1H), 7.25 (t, 1H), 6.84 (d, 2H), 3.72 (s, 3H), 3.20 (br d, 2H), 3.12 (s, 3H), 0.93 (t, 3H); LCMS method A, (ES+) 432, RT=2.45 min.

### Example 157

### N-(2-(2-(3,4-dimethoxyphenylamino)-5-fluoropyrimidin-4-ylamino)phenyl)-N-ethylmethanesulfonamide

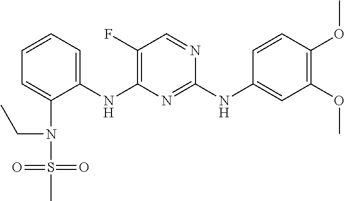

Synthesized according to the procedure in Example 1 using Intermediate 1j and the appropriate aniline derivative. ¹H NMR (d₆-DMSO) δ 9.14 (s, 1H), 8.40 (d, 1H), 8.17 (d, 1H), 7.98 (br s, 1H), 7.62 (d, 1H), 7.43 (t, 2H), 7.22-7.27 (m, 2H), 7.16 (d, 1H), 6.83 (d, 1H), 3.71 (s, 3H), 3.62 (s, 3H), 3.20 (br d, 2H), 3.12 (s, 3H), 0.94 (t, 3H); LCMS method A, (ES+) 462, RT=2.55 min.

### Example 158

### N-ethyl-N-(2-(5-fluoro-2-(4-(2-(piperidin-1-yl)ethoxy)phenylamino)pyrimidin-4-ylamino)phenyl)methanesulfonamide

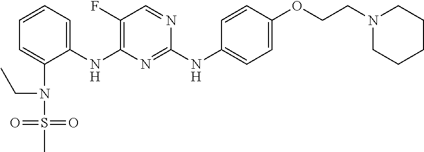

Synthesized according to the procedure in Example 1 using Intermediate 1j and the appropriate aniline derivative. ¹H NMR (d₆-DMSO) δ 9.15 (s, 1H), 8.40 (d, 1H), 8.17 (s, 2H), 8.15 (d, 1H), 7.98 (br s, 1H), 7.61 (d, 1H), 7.50 (d, 2H), 7.44 (t, 1H), 7.24 (t, 1H), 6.85 (d, 2H), 4.05 (t, 2H), 3.20 (br d, 2H), 3.12 (s, 3H), 2.68 (t, 2H), 2.48 (t, 2H), 1.49-1.55 (m, 4H), 1.38-1.41 (m, 2H), 0.93 (t, 3H); LCMS method A, (ES+) 529, RT=2.35 min.

### Example 159

### N-ethyl-N-(2-(5-fluoro-2-(4-(2-morpholinoethoxy)phenylamino)pyrimidin-4-ylamino)phenyl)methanesulfonamide

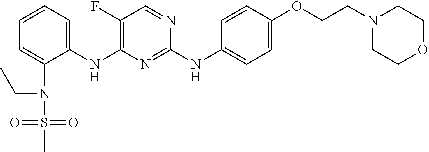

Synthesized according to the procedure in Example 1 using Intermediate 1j and the appropriate aniline derivative. ¹H NMR (d₆-DMSO) δ 9.15 (s, 1H), 8.40 (d, 1H), 8.16 (s, 1H), 7.98 (br s, 1H), 7.61 (d, 1H), 7.54 (d, 2H), 7.43 (t, 1H), 7.25 (t, 1H), 6.84 (d, 2H), 4.02-4.04 (m 2H), 3.57-3.59 (m, 4H), 3.20 (br d, 2H), 3.12 (s, 3H), 2.68 (t, 2H), 2.48 (t, 2H), 0.92 (t, 3H); LCMS method A, (ES+) 531, RT=2.30 min.

### Example 160

### N-(2-(5-chloro-2-(3,4,5-trimethoxyphenylamino)pyrimidin-4-ylamino)-6-methylphenyl)methanesulfonamide

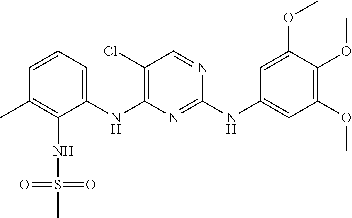

Synthesized according to the procedure in Example 1 using the appropriate 2-chloropyrimidine and aniline derivatives. ¹H NMR (d₆-DMSO) δ 9.25 (s, 1H), 9.09 (s, 1H), 8.60 (s, 1H), 8.20 (s, 1H), 7.74 (d, 1H), 7.20 (t, 1H), 7.11 (d, 1H), 6.94 (s, 2H), 3.57 (s, 3H), 3.51 (s, 6H), 2.96 (s, 3H), 2.38 (s, 3H); LCMS method A, (ES+) 494, RT=2.37 min.

### Example 161

### N-(2-(5-fluoro-2-(3,4,5-trimethoxyphenylamino)pyrimidin-4-ylamino)-6-isopropylphenyl)methanesulfonamide

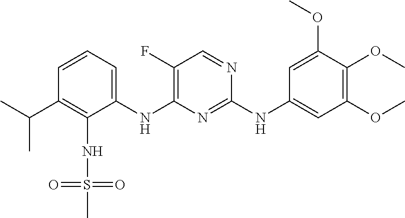

Synthesized according to the procedure in Example 1 using the appropriate 2-chloropyrimidine and aniline derivatives. ¹H NMR (d₆-DMSO) δ 9.13 (s, 1H), 8.76 (s, 1H), 8.16 (d, 1H), 7.59 (d, 1H), 7.30 (t, 1H), 7.23 (d, 1H), 6.96 (s, 2H), 3.56-3.51 (m, 4H), 3.45 (s, 6H), 2.88 (s, 3H), 1.16 (d, 6H); LCMS method B, (ES+) 506, RT=8.64 min.

### Example 162

### N-(3-fluoro-6-(5-fluoro-2-(3,4,5-trimethoxyphenylamino)pyrimidin-4-ylamino)-2-methylphenyl)methanesulfonamide

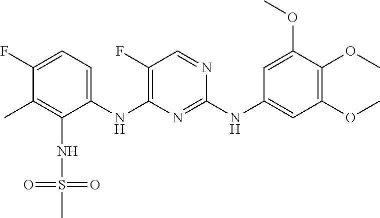

Synthesized according to the procedure in Example 1 using the appropriate 2-chloropyrimidine and aniline derivatives. ¹H NMR (d₆-DMSO) δ 9.08 (s, 1H), 8.74 (s, 1H), 8.14 (d, 1H), 7.60 (dd, 1H), 7.18 (t, 1H), 6.96 (s, 2H), 3.56 (s, 3H), 3.51 (s, 6H), 2.92 (s, 3H), 2.27 (d, 3H); LCMS method B, (ES+) 496, RT=8.07 min.

### Example 163

### N-(6-(5-fluoro-2-(3,4,5-trimethoxyphenylamino)pyrimidin-4-ylamino)-3-methoxy-2-methylphenyl)methanesulfonamide

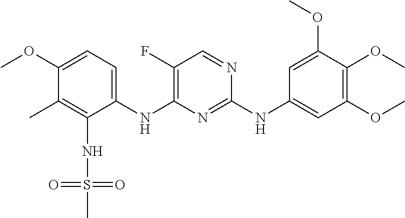

Synthesized according to the procedure in Example 1 using the appropriate 2-chloropyrimidine and aniline derivatives. ¹H NMR (d₆-DMSO) δ 9.03 (s, 1H), 8.63 (s, 1H), 8.10 (d, 1H), 7.46 (d, 1H), 6.98-6.95 (m, 3H), 3.82 (s, 3H), 3.55 (s, 3H), 3.47 (s, 6H), 2.87 (s, 3H), 2.19 (s, 3H); LCMS method B, (ES+) 508, RT=7.38 min.

### Example 164

### N-(6-(5-chloro-2-(3,4,5-trimethoxyphenylamino)pyrimidin-4-ylamino)-2,3-dihydrobenzo[b][1,4]dioxin-5-yl)methanesulfonamide

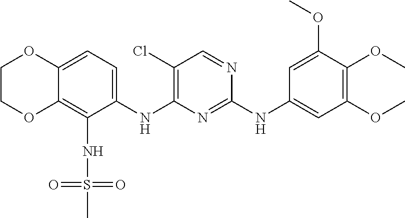

Synthesized according to the procedure in Example 1 using the appropriate 2-chloropyrimidine and aniline derivatives. ¹H NMR (d₆-DMSO) δ 9.81 (br s, 1H), 9.21 (s, 1H), 8.84 (br s, 1H), 8.26 (s, 1H), 7.39 (d, 1H), 6.85 (s, 2H), 6.80 (d, 1H), 4.32 (dt, 4H), 3.61 (s, 3H), 3.59 (s, 6H), 3.02 (s, 3H); LCMS method B, (ES+) 538, RT=8.37 min.

### Example 165

### N-(6-(5-fluoro-2-(3,4,5-trimethoxyphenylamino)pyrimidin-4-ylamino)-2,3-dimethylphenyl)methanesulfonamide

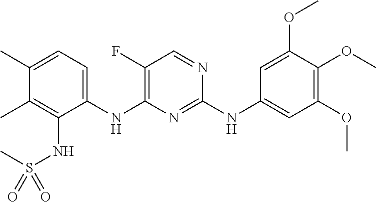

Synthesized according to the procedure in Example 1 using the appropriate 2-chloropyrimidine and aniline derivatives. ¹H NMR: (d₆-DMSO) δ 9.09 (s, 1H), 8.80 (s, 1H), 8.68 (s, 1H), 8.14 (d, 1H), 7.50 (d, 1H), 7.14 (d, 1H), 6.97 (s, 2H), 3.56 (s, 3H), 3.48 (s, 6H), 2.88 (s, 3H), 2.27 (s, 6H); LCMS method A, (ES+) 492, RT=3.02 min.

### Example 166

### N-(2-ethoxy-6-(5-fluoro-2-(3,4,5-trimethoxyphenylamino)pyrimidin-4-ylamino)phenyl)methanesulfonamide

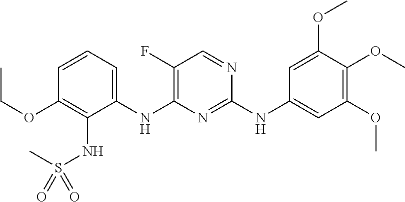

Synthesized according to the procedure in Example 1 using the appropriate 2-chloropyrimidine and aniline derivatives. ¹H NMR: (d₆-DMSO) δ 9.17 (s, 1H), 9.03 (s, 1H), 8.38 (d, 1H), 8.18 (d, 1H), 7.89 (d, 1H), 7.26 (t, 1H), 7.03 (s, 2H), 4.11 (q, 2H), 3.64 (s, 6H), 3.60 (s, 3H), 3.01 (s, 3H), 1.39 (t, 3H); LCMS method A, (ES+) 508, RT=2.35 min.

### Example 167

### N-(6-(5-fluoro-2-(3,4,5-trimethoxyphenylamino)pyrimidin-4-ylamino)-2,3-dihydrobenzo[b][1,4]dioxin-5-yl)methanesulfonamide

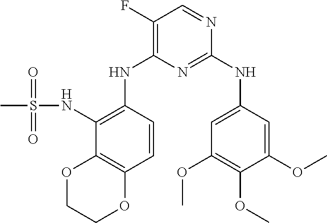

Synthesized according to the procedure in Example 1 using the appropriate 2-chloropyrimidine and aniline derivatives. ¹H NMR (d₆-DMSO) δ 9.08 (s, 1H), 8.30 (s, 1H), 8.12 (d, 1H), 7.48 (d, 1H), 7.01 (s, 2H), 6.84 (d, 1H), 4.35-4.33 (m, 2H), 4.29-4.28 (m, 2H), 3.60 (s, 6H), 3.59 (s, 3H), 3.02 (s, 3H); LCMS method A, (ES+) 522, RT=2.07 min.

### Example 168

### N-(3-ethoxy-6-(5-fluoro-2-(3,4,5-trimethoxyphenylamino)pyrimidin-4-ylamino)-2-methylphenyl)methanesulfonamide

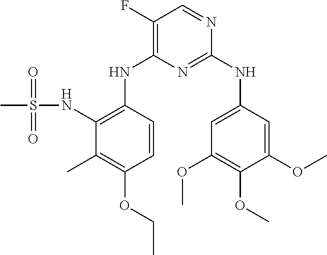

Synthesized according to the procedure in Example 1 using the appropriate 2-chloropyrimidine and aniline derivatives. ¹H NMR (d₆-DMSO) δ 9.03 (s, 1H), 8.66 (s, 1H), 8.10 (d, 1H), 7.46 (d, 1H), 6.97 (s, 2H), 6.93 (d, 1H), 4.06 (quartet, 2H), 3.55 (s, 3H), 3.49 (s, 6H), 2.87 (s, 3H), 2.20 (s, 3H), 1.37 (t, 3H); LCMS method B, (ES+) 522, RT=8.05 min.

### Example 169

### N-(2-ethyl-6-(5-fluoro-2-(3,4,5-trimethoxyphenylamino)pyrimidin-4-ylamino)phenyl)methanesulfonamide

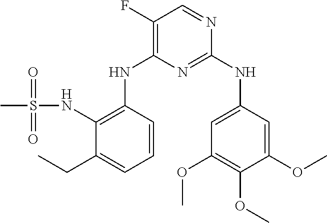

Synthesized according to the procedure in Example 1 using the appropriate 2-chloropyrimidine and aniline derivatives. ¹H NMR (d₆-DMSO) δ 9.13 (s, 1H), 8.77 (s, 1H), 8.16 (d, 1H), 7.64 (d, 1H), 7.27 (dd, 1H), 7.17 (d, 1H), 6.97 (s, 2H), 3.56 (s, 3H), 3.49 (s, 6H), 2.90 (s, 3H), 2.79 (quartet, 2H), 1.17 (t, 3H); LCMS method B, (ES+) 492, RT=8.29 min.

### Example 170

### N-(2-(5-Fluoro-2-(3,4,5-trimethoxyphenylamino)pyrimidin-4-ylamino)-5-methylphenyl)methanesulfonamide

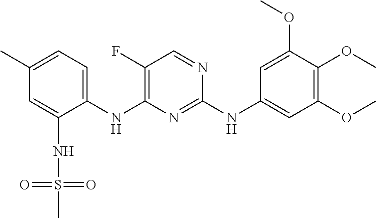

Synthesized according to the procedure in Example 1 using the appropriate 2-chloropyrimidine and aniline derivatives. ¹H NMR (d₆-DMSO) δ 9.10 (s, 1H), 9.01 (s, 1H), 8.61 (s, 1H), 8.10 (d, 1H), 7.56 (d, 1H), 7.22 (s, 1H), 7.05 (dd, 1H), 6.95 (s, 2H), 3.55 (s, 3H), 3.50 (s, 6H), 2.90 (s, 3H), 2.31 (3H); LCMS method A, (ES+) 478, RT=2.18 min.

### Example 171

### N-(2-(5-fluoro-2-(3,4,5-trimethoxyphenylamino)pyrimidin-4-ylamino)-5-morpholinophenyl)methanesulfonamide

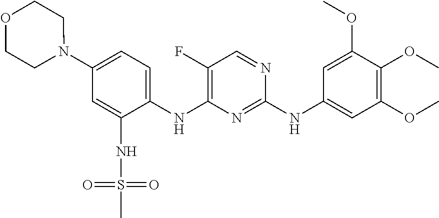

Synthesized according to the procedure in Example 1 using the appropriate 2-chloropyrimidine and aniline derivatives. ¹H NMR (d₆-DMSO) δ 8.97 (s, 1H), 8.54 (s, 1H), 8.06 (d, 1H), 7.46-7.47 (m, 1H), 6.97 (s, 1H), 6.94 (d, 2H), 6.81-6.83 (m, 1H), 3.76 (t, 4H), 3.55 (s, 3H), 3.51 (s, 6H), 3.10 (t, 4H), 2.88 (s, 3H); LCMS method A, (ES+) 548, RT=2.04 min.

### Example 172

### N-(2-(5-fluoro-2-(3,4,5-trimethoxyphenylamino)pyrimidin-4-ylamino)-6-methoxyphenyl)methanesulfonamide

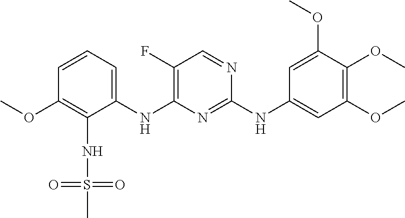

Synthesized according to the procedure in Example 1 using the appropriate 2-chloropyrimidine and aniline derivatives. ¹H NMR (d₆-DMSO) δ 9.18 (s, 1H), 9.06 (s, 1H), 8.40 (s, 1H), 8.18 (d, 1H), 7.89 (d, 1H), 7.28 (t, 1H), 7.03 (s, 2H), 6.91 (d, 1H), 3.87 (s, 3H), 3.63 (s, 6H), 3.60 (s, 3H), 2.99 (s, 3H); LCMS method A, (ES+) 494, RT=2.24 min.

### Example 173

### N-(2-(5-fluoro-2-(3,4,5-trimethoxyphenylamino)pyrimidin-4-ylamino)-5-methoxyphenyl)methanesulfonamide

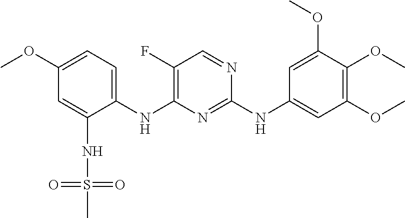

Synthesized according to the procedure in Example 1 using the appropriate 2-chloropyrimidine and aniline derivatives. ¹H NMR (d₆-DMSO) δ 9.30 (s, 1H), 8.22 (d, 1H), 7.35 (d, 1H), 7.05 (d, 1H), 6.81 (dd, 1H), 6.72 (s, 2H), 3.78 (s, 3H), 3.58 (s, 3H), 3.52 (s, 6H), 2.93 (s, 3H); LCMS method A, (ES+) 494, RT=2.05 min.

### Example 174

### N-(4,5-difluoro-2-(5-fluoro-2-(3,4,5-trimethoxyphenylamino)pyrimidin-4-ylamino)phenyl)methanesulfonamide

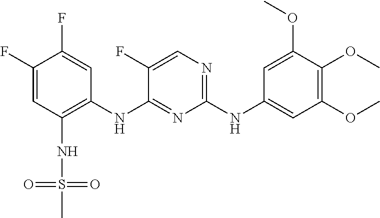

Synthesized according to the procedure in Example 1 using the appropriate 2-chloropyrimidine and aniline derivatives. ¹H NMR (MeOD) δ 8.23 (br s, 2H), 8.02-8.08 (m, 2H), 7.41-7.45 (m, 1H), 6.89 (s, 2H), 3.74 (s, 9H), 2.98 (s, 3H); LCMS method A, (ES+) 500, RT=2.42 min.

### Example 175

### N-(5-ethoxy-2-(5-fluoro-2-(3,4,5-trimethoxyphenylamino)pyrimidin-4-ylamino)phenyl)methanesulfonamide

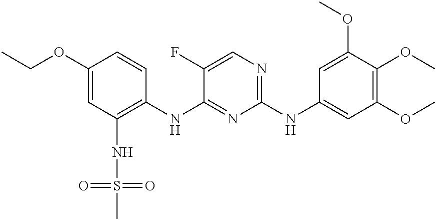

Synthesized according to the procedure in Example 1 using the appropriate 2-chloropyrimidine and aniline derivatives. ¹H NMR (d₆-DMSO) δ 9.14 (s, 1H), 8.94 (s, 1H), 8.55 (s, 1H), 8.06 (d, 1H), 7.44 (d, 1H), 6.99 (d, 1H), 6.95 (s, 2H), 6.81 (dd, 1H), 4.03 (q, 2H), 3.56 (s, 3H), 3.51 (s, 6H), 2.90 (s, 3H), 1.36 (t, 3H); LCMS method A, (ES+) 508, RT=2.21 min.

### Example 176

### N-(2-methyl-6-(2-(3,4,5-trimethoxyphenylamino)pyrimidin-4-ylamino)phenyl)methanesulfonamide

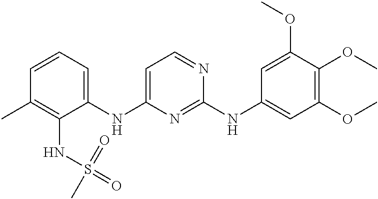

Synthesized according to the procedure in Example 1 using the appropriate 2-chloropyrimidine and aniline derivatives. ¹H NMR (d₆-DMSO) δ 9.07 (s, 1H), 8.71 (s, 1H), 8.04 (d, 1H), 7.53 (d, 1H), 7.20 (t, 1H), 7.09 (s, 1H), 7.07 (s, 1H), 6.24 (d, 1H), 3.58 (s, 9H), 2.89 (s, 3H), 2.37 (s, 3H); LCMS method C, (ES+) 460, RT=2.16 min.

### Example 177

### N-(2-(2-(3-methoxy-5-(2-(piperidin-1-yl)ethoxy)phenylamino)pyrimidin-4-ylamino)-6-methylphenyl)methanesulfonamide formate salt

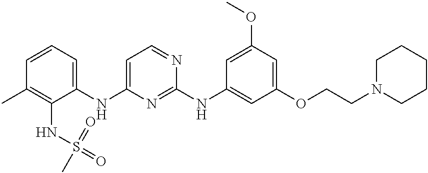

Synthesized according to the procedure in Example 1 using the appropriate 2-chloropyrimidine and aniline derivatives. ¹H NMR (d₆-DMSO) δ 9.12 (s, 1H), 8.81 (s, 1H), 8.23 (br s, 2H), 8.04 (d, 1H), 7.60 (d, 1H), 7.23 (t, 1H), 7.08 (d, 1H), 6.95-7.00 (m, 2H), 6.28 (d, 1H), 6.05 (t, 1H), 3.89 (t, 2H), 3.61 (s, 3H), 2.88 (s, 3H), 2.62 (t, 2H), 2.40-2.47 (m, 4H), 2.37 (s, 3H), 1.45-1.55 (m, 4H), 1.30-1.42 (m, 2H); LCMS method C, (ES+) 527, RT=2.55 min.

### Example 178

### N-(2-(5-bromo-2-(3,4,5-trimethoxyphenylamino)pyrimidin-4-ylamino)-5-methoxyphenyl)methanesulfonamide

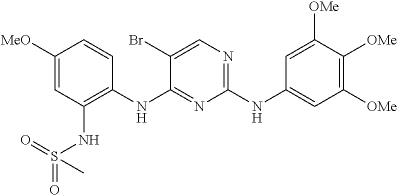

Synthesized according to the procedure in Example 1 using the appropriate 2-chloropyrimidine and aniline derivatives. ¹H NMR (d₆-DMSO) δ 9.37 (br s, 1H), 9.20 (br s, 1H), 8.30 (s, 1H), 8.26 (s, 1H), 7.66 (d, 1H), 7.00 (d, 1H), 6.97 (s, 2H), 6.89 (d, 1H), 3.84 (s, 3H), 3.62 (s, 3H), 3.61 (s, 6H), 2.99 (s, 3H); LCMS method B, (ES+) 554/556, RT=8.51 min.

### Example 179

### N-(2-(5-bromo-2-(3,4-dimethoxyphenylamino)pyrimidin-4-ylamino)-5-methoxyphenyl)methanesulfonamide

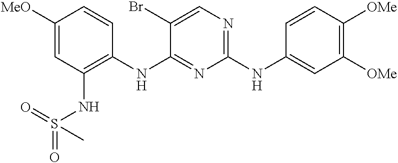

Synthesized according to the procedure in Example 1 using the appropriate 2-chloropyrimidine and aniline derivatives. ¹H NMR (d₆-DMSO) δ 9.16 (br s, 1H), 8.98 (br s, 1H), 8.13 (br s, 1H), 8.05 (s, 1H), 7.52 (d, 1H), 7.02 (s, 1H), 6.95 (d, 1H), 6.85 (d, 1H), 6.76 (m, 1H), 6.57 (d, 1H), 3.68 (s, 3H), 3.56 (s, 3H), 3.40 (s, 3H), 2.80 (s, 3H); LCMS method B, (ES+) 524/526, RT=7.74 min.

### Example 180

### N-(2-(2-(3,4-dimethoxyphenylamino)-5-fluoropyrimidin-4-ylamino)-5-methoxyphenyl)methanesulfonamide

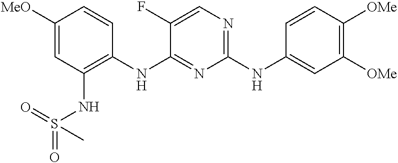

Synthesized according to the procedure in Example 1 using the appropriate 2-chloropyrimidine and aniline derivatives. ¹H NMR (d₆-DMSO) δ 8.89 (br s, 1H), 8.56 (br s, 1H), 8.15 (s, 1H), 8.04 (d, 1H), 7.51 (d, 1H), 7.22 (d, 1H), 7.07 (d, 2H), 7.01 (d, 1H), 6.85-6.81 (m, 1H), 6.70 (d, 1H), 3.78 (s, 3H), 3.66 (s, 3H), 3.50 (s, 3H), 2.90 (s, 3H); LCMS method B, (ES+) 464, RT=6.54 min.

### Example 181

### N-(2-(5-fluoro-2-(3,4,5-trimethoxyphenylamino)pyrimidin-4-ylamino)-6-methylphenyl)-N-methylmethanesulfonamide

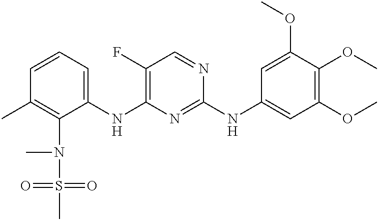

Synthesized according to the procedure in Example 1 using the appropriate 2-chloropyrimidine and aniline derivatives. ¹H NMR (d₆-DMSO) δ 9.12 (s, 1H), 8.72 (s, 1H), 8.13 (d, 1H), 7.54 (d, 1H), 7.29 (t, 1H), 7.20 (d, 1H), 6.94 (s, 2H), 3.55 (s, 3H), 3.49 (s, 6H), 3.10 (s, 3H), 3.08 (s, 3H), 2.34 (s, 3H); LCMS method B, (ES+) 492, RT=8.31 min.

### Example 182

### N-(2-(5-fluoro-2-(3,4,5-trimethoxyphenylamino)pyrimidin-4-ylamino)-5-isopropoxyphenyl)methanesulfonamide

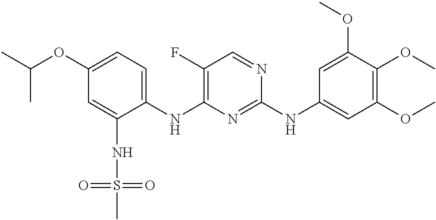

Synthesized according to the procedure in Example 1 using the appropriate 2-chloropyrimidine and aniline derivatives. ¹H NMR (d₆-DMSO) δ 9.13 (s, 1H), 8.95 (s, 1H), 8.55 (s, 1H), 8.06 (d, 1H), 7.42 (d, 1H), 6.96-6.94 (m, 3H), 6.78 (dd, 1H), 4.57 (septet, 1H), 3.54 (s, 3H), 3.50 (s, 6H), 2.90 (s, 3H), 1.29 (d, 6H); LCMS method B, (ES+) 522, RT=8.31 min.

### Example 183

### N-(2-fluoro-6-(5-fluoro-2-(3,4,5-trimethoxyphenylamino)pyrimidin-4-ylamino)phenyl)methanesulfonamide

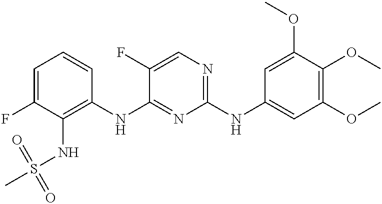

Synthesized according to the procedure in Example 1 using the appropriate 2-chloropyrimidine and aniline derivatives. ¹H NMR (d₆-DMSO) δ 9.36 (s, 1H), 9.19 (s, 1H), 8.60 (d, 1H), 8.21 (d, 1H), 7.96 (d, 1H), 7.36 (dd, 1H), 7.16 (dd, 1H), 7.01 (s, 2H), 3.61 (s, 6H), 3.59 (s, 3H), 3.02 (s, 3H); LCMS method A, (ES+) 482 RT=2.28 min.

### Example 184

### N-(2-chloro-6-(5-fluoro-2-(3,4,5-trimethoxyphenylamino)pyrimidin-4-ylamino)phenyl)methanesulfonamide

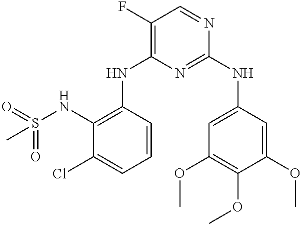

Synthesized according to the procedure in Example 1 using the appropriate 2-chloropyrimidine and aniline derivatives. ¹H NMR (MeOD) δ 8.28 (br s, 1H), 8.20 (dd, 1H), 8.03 (d, 1H), 7.30-7.36 (m, 2H), 6.92 (s, 2H), 3.79 (s, 3H), 3.71 (s, 6H), 3.11 (s, 3H); LCMS method B, (ES+) 498, 500, RT=8.50 min.

### Example 185

### N-(2-(5-fluoro-2-(3,4,5-trimethoxyphenylamino)pyrimidin-4-ylamino)-5-(3-(piperidin-1-yl)propoxy)phenyl)methanesulfonamide

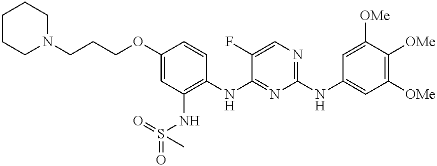

Synthesized according to the procedure in Example 1 using the appropriate 2-chloropyrimidine and aniline derivatives. ¹H NMR (d₆-DMSO) δ 8.97 (br s, 1H), 8.63 (br s, 1H), 8.20 (s, 2H), 8.05 (d, 1H), 7.56 (d, 1H), 6.96 (m, 2H), 6.69 (m, 1H), 3.98 (t, 2H), 3.56 (s, 3H), 3.52 (s, 6H), 2.86 (s, 3H), 2.48-2.39 (m, 6H), 1.94-1.87 (m, 2H), 1.56-1.49 (m, 4H), 1.44-1.36 (m, 2H); LCMS method B, (ES+) 605, 303, RT=5.16 min.

### Example 186

### N-(2-(5-fluoro-2-(3,4,5-trimethoxyphenylamino)pyrimidin-4-ylamino)-4,6-dimethylphenyl)methanesulfonamide

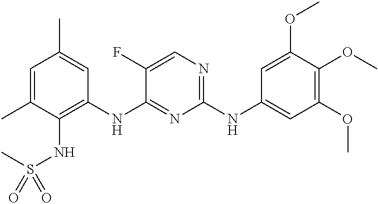

Synthesized according to the procedure in Example 1 using the appropriate 2-chloropyrimidine and aniline derivatives. ¹H NMR: (d₆-DMSO) δ 9.11 (s, 1H), 8.77 (br s, 1H), 8.67 (s, 1H), 8.15 (d, 1H), 7.51 (s, 1H), 6.96 (s, 2H), 6.94 (s, 1H), 3.58 (s, 3H), 3.52 (s, 6H), 2.91 (s, 3H), 2.34 (s, 3H), 2.21 (s, 3H); LCMS method A, (ES+) 492, RT=2.27 min.

### Example 187

### N-(6-(5-chloro-2-(3,4,5-trimethoxyphenylamino)pyrimidin-4-ylamino)-2-fluoro-3-methoxyphenyl)methanesulfonamide

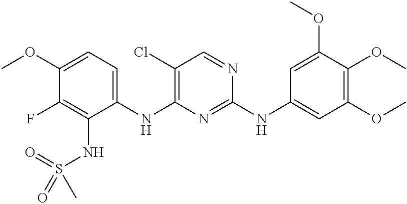

Synthesized according to the procedure in Example 1 using the appropriate 2-chloropyrimidine and aniline derivatives. ¹H NMR (d₆-DMSO) δ 9.43 (br. s, 1H), 9.19 (s, 1H), 8.37 (s, 1H), 8.18 (s, 1H), 7.69 (d, 1H), 7.13 (t, 1H), 6.95 (s, 2H), 3.87 (s, 3H), 3.59 (s, 3H), 3.57 (s, 6H), 3.01 (s, 3H); LCMS method B, (ES+) 528, RT=9.10 min.

### Example 188

### N-(2-(2-(3-(1H-tetrazol-1-yl)phenylamino)-5-fluoropyrimidin-4-ylamino)phenyl)methanesulfonamide

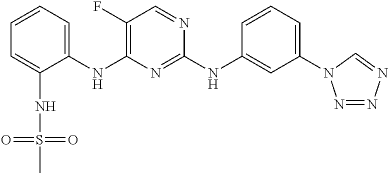

Synthesized according to the procedure in Example 1 using Intermediate 1a and the appropriate aniline derivative. ¹H NMR (d₆-DMSO) δ 9.13 (br s, 1H), 9.63 (br s, 1H), 9.24 (br s, 1H), 8.79 (br s, 1H), 7.75 (d, 2H), 7.67 (d, 1H) 7.49 (t, 2H), 7.32 (d, 1H) 7.07-7.13 (m, 2H), 2.94 (s, 1H); LCMS method A, (ES+) 442, RT=2.24 min.

### Example 189

### N-(2-(5-fluoro-2-(3-(5-methyl-1,2,4-oxadiazol-3-yl)phenylamino)pyrimidin-4-ylamino)phenyl)methanesulfonamide

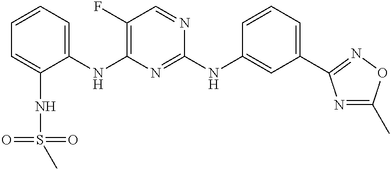

Synthesized according to the procedure in Example 1 using Intermediate 1a and the appropriate aniline derivative. ¹H NMR (d₆-DMSO) δ 9.63 (br s, 1H), 9.32 (br s, 1H), 9.05 (br s, 1H), 7.95 (d, 2H), 7.55 (s, 1H), 7.46 (s, 1H), 7.16 (d, 2H) 7.08 (d, 1H), 3.35 (s, 3H), 2.72 (s, 3H); LCMS method A, (ES+) 456, RT=2.32 min.

### Example 190

### N-(2-(2-(3-(1H-tetrazol-5-yl)phenylamino)-5-fluoropyrimidin-4-ylamino)phenyl)methanesulfonamide

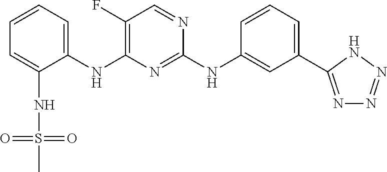

¹H NMR (d₆-DMSO) δ 9.53 (br s, 1H), 9.41 (br s, 1H), 8.90 (br s, 1H), 7.95 (d, 2H), 7.55 (s, 1H), 7.46 (s, 1H), 7.16 (d, 2H) 7.08 (d, 1H), 3.35 (s, 3H); LCMS method A, (ES+) 442, RT=2.05 min.

### Example 191

### N-(2-(5-fluoro-2-(4-(3-methyl-1H-1,2,4-triazol-5-yl)phenylamino)pyrimidin-4-ylamino)phenyl)methanesulfonamide

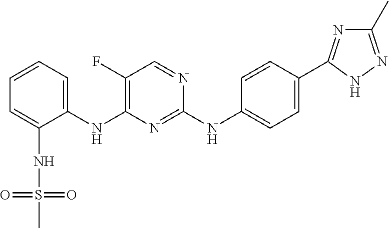

Synthesized according to the procedure in Example 1 using Intermediate 1a and the appropriate aniline derivative. ¹H NMR (d₆-DMSO) δ 9.38 (s, 1H), 8.35 (d, 1H), 7.91 (br s, 1H), 7.76 (d, 2H), 7.51 (t, 1H), 7.40-7.46 (m, 2H), 7.20-7.26 (m, 2H), 7.12 (t, 1H), 3.35 (s, 3H), 2.90 (s, 3H); LCMS method A, (ES+) 446, RT=2.12 min.

### Example 192

### N-(2-(5-fluoro-2-(4-(2-methylthiazol-4-yl)phenylamino)pyrimidin-4-ylamino)phenyl)methanesulfonamide

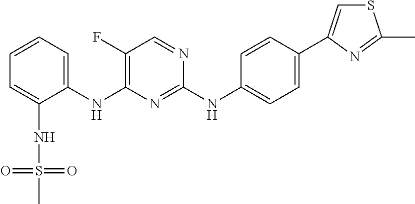

Synthesized according to the procedure in Example 1 using Intermediate 1a and the appropriate aniline derivative. ¹H NMR (d₆-DMSO) δ 10.10 (br s, 1H), 9.82 (br s, 1H), 9.32 (s, 1H), 8.31 (d, 1H), 7.92 (br s, 1H), 7.66 (br s, 2H), 7.55 (d, 1H), 7.47 (d, 1H), 7.40 (d, 1H), 7.26 (d, 1H), 7.19 (t, 1H), 7.12 (t, 1H), 2.95 (s, 3H), 2.71 (s, 3H); LCMS method A, (ES+) 471, RT=2.26 min.

### Example 193

### N-(2-(5-fluoro-2-(3-(oxazol-5-yl)phenylamino)pyrimidin-4-ylamino)phenyl)methanesulfonamide

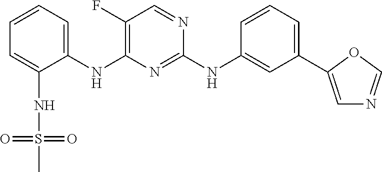

Synthesized according to the procedure in Example 1 using Intermediate 1a and the appropriate aniline derivative. ¹H NMR (d₆-DMSO) δ 10.22 (br s, 1H), 9.83 (br s, 1H), 9.33 (s, 1H), 8.41 (s, 1H), 8.32 (d, 1H), 7.53 (br s, 1H), 7.64 (d, 1H), 7.49 (s, 1H), 7.48 (d, 1H), 7.40 (d, 1H), 7.35 (d, 1H), 7.25 (t, 1H), 7.14 (t, 1H), 2.93 (s, 3H); LCMS method A, (ES+) 441, RT=2.31 min.

### Example 194

### N-(2-(5-fluoro-2-(3-(5-methyl-1H-tetrazol-1-yl)phenylamino)pyrimidin-4-ylamino)phenyl)methanesulfonamide

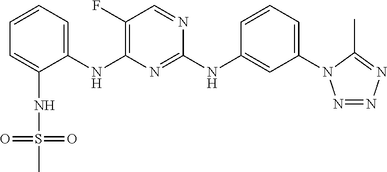

Synthesized according to the procedure in Example 1 using Intermediate 1a and the appropriate aniline derivative. ¹H NMR (d₆-DMSO) δ 9.65 (br s, 1H), 9.21 (br s, 1H), 8.83 (s, 1H), 8.18 (d, 1H), 7.94 (s, 1H), 7.67 (t, 2H), 7.34-7.41 (m, 2H), 7.03-7.09 (m, 2H), 6.95 (t, 1H), 2.91 (s, 3H), 2.41 (s 3H); LCMS method A, (ES+) 456, RT=2.29 min.

### Example 195

### N-(2-(2-(4-(1H-tetrazol-1-yl)phenylamino)-5-fluoropyrimidin-4-ylamino)phenyl)methanesulfonamide

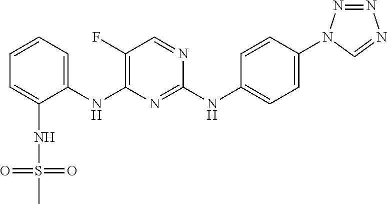

Synthesized according to the procedure in Example 1 using Intermediate 1a and the appropriate aniline derivative. ¹H NMR (d₆-DMSO) δ 9.97 (br s, 1H), 9.61 (br s, 1H), 9.25 (br s, 1H), 8.83 (s, 1H), 8.20 (d, 1H), 7.78-7.83 (m, 2H), 7.64 (d, 2H), 7.49 (d, 2H), 7.28-7.38 (m, 2H), 2.94 (s, 3H); LCMS method A, (ES+) 442, RT=2.25 min.

### Example 196

### N-(2-(2-(4-cyanophenylamino)-5-fluoropyrimidin-4-ylamino)phenyl)methanesulfonamide

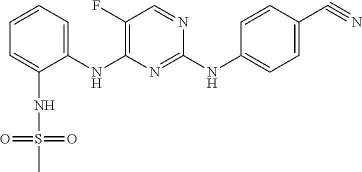

Synthesized according to the procedure in Example 1 using Intermediate 1a and the appropriate aniline derivative. ¹H NMR (d₆-DMSO) δ 10.19 (br s, 1H), 9.61 (br s, 1H), 9.35 (s, 1H), 8.29 (d, 1H), 7.94 (s, 1H), 7.65 (t, 2H), 7.51 (t, 1H), 7.32-7.36 (m, 4H), 2.93 (s, 3H); LCMS method A, (ES+) 399, RT=2.33 min.

### Example 197

### N-(2-(5-fluoro-2-(4-(5-methyl-1,2,4-oxadiazol-3-yl)phenylamino)pyrimidin-4-ylamino)phenyl)methanesulfonamide

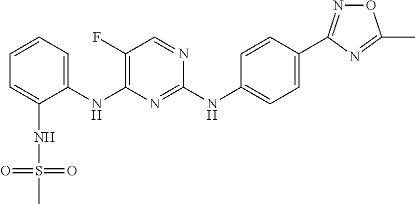

Synthesized according to the procedure in Example 1 using Intermediate 1a and the appropriate aniline derivative. ¹H NMR (d₆-DMSO) δ 9.47 (br s, 1H), 9.25 (br s, 1H), 8.74 (br s, 1H), 8.24 (br s, 1H), 8.19 (d, 1H), 7.81-7.85 (m, 2H), 7.48 (d, 1H), 7.42-7.45 (m, 1H), 7.29 (t, 2H), 7.21 (t, 2H), 2.94 (s, 3H), 2.65 (s, 3H); LCMS method A, (ES+) 456, RT=2.32 min.

### Example 198

### N-(2-(2-(4-(1H-1,2,4-triazol-1-yl)phenylamino)-5-fluoropyrimidin-4-ylamino)phenyl)methanesulfonamide

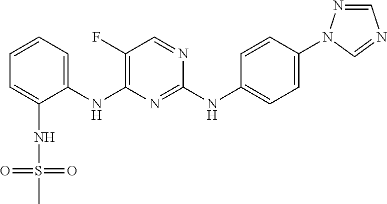

Synthesized according to the procedure in Example 1 using Intermediate 1a and the appropriate aniline derivative. ¹H NMR (d₆-DMSO) δ 9.74 (s, 1H), 8.79 (br s, 1H), 8.14-8.18 (m, 2H), 7.83 (d, 1H), 7.58 (d, 2H), 7.48 (d, 2H), 7.29-7.35 (m, 2H), 2.93 (s, 3H); LCMS method A, (ES+) 441, RT=2.33 min.

### Example 199

### N-(2-(5-fluoro-2-(4-(5-methyl-1H-tetrazol-1-yl)phenylamino)pyrimidin-4-ylamino)phenyl)methanesulfonamide

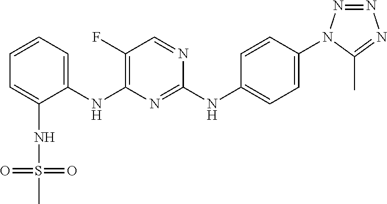

Synthesized according to the procedure in Example 1 using Intermediate 1a and the appropriate aniline derivative. ¹H NMR (d₆-DMSO) δ 9.62 (br s, 1H), 8.85 (br s, 1H), 8.19 (d, 1H), 7.81-7.83 (m, 3H), 7.47 (d, 1H), 7.42 (d, 2H), 7.26-7.31 (m, 2H), 2.92 (s, 3H), 2.52 (s, 3H); LCMS method A, (ES+) 456, RT=2.37 min.

### Example 200

### N-(2-(2-(3-cyanophenylamino)-5-fluoropyrimidin-4-ylamino)phenyl)methanesulfonamide

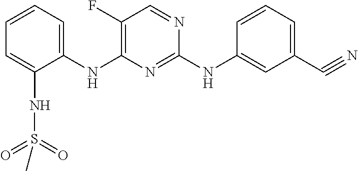

Synthesized according to the procedure in Example 1 using Intermediate 1a and the appropriate aniline derivative. ¹H NMR (d₆-DMSO) δ 10.19 (br s, 1H), 9.61 (br s, 1H), 9.35 (s, 1H), 8.29 (d, 1H), 7.94 (s, 1H), 7.65 (t, 2H), 7.51 (t, 1H), 7.32-7.36 (m, 4H), 2.93 (s, 3H); LCMS method A, (ES+) 399, RT=2.33 min.

### Example 201

### N-(2-(5-fluoro-2-(3-(1-methyl-1H-pyrazol-3-yl)phenylamino)pyrimidin-4-ylamino)phenyl)methanesulfonamide

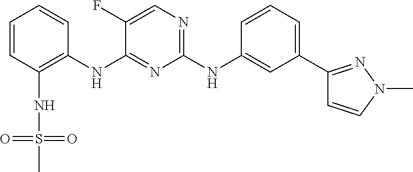

Synthesized according to the procedure in Example 1 using Intermediate 1a and the appropriate aniline derivative. ¹H NMR (d₆-DMSO) δ 9.23 (s, 1H), 8.67 (s, 1H), 8.14 (s, 2H), 7.91 (d, 2H), 7.70 (d, 1H), 7.59 (d, 1H), 7.41 (d, 1H), 7.27 (d, 1H), 7.12-7.20 (m, 3H), 6.46 (s, 1H), 3.86 (s, 3H), 2.93 (s, 3H); LCMS method A, (ES+) 454, RT=2.13 min.

### Example 202

### N-(2-(2-(3-(2,5-dimethyl-1H-pyrrol-1-yl)phenylamino)-5-fluoropyrimidin-4-ylamino)phenyl)methanesulfonamide

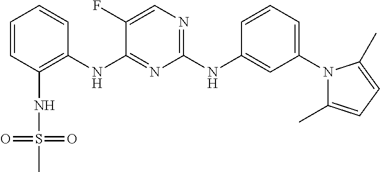

Synthesized according to the procedure in Example 1 using Intermediate 1a and the appropriate aniline derivative. ¹H NMR (d₆-DMSO) δ 9.37 (s, 1H), 9.18 (s, 1H), 8.72 (s, 1H), 8.16 (d, 1H), 7.74 (d, 1H), 7.62 (dd, 1H), 7.55 (s, 1H), 7.40 (dd, 1H), 7.12-7.26 (m, 3H), 6.68 (d, 1H), 5.78 (s, 2H), 2.92 (s, 3H), 1.92 (s, 6H); LCMS method B, (ES+) 467, RT=10.96 min.

### Example 203

### N-(2-(2-(3-(1H-tetrazol-1-yl)phenylamino)-5-chloropyrimidin-4-ylamino)phenyl)methanesulfonamide

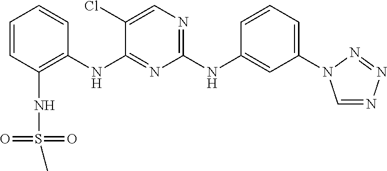

Synthesized according to the procedure in Example 1 using Intermediate 1h and the appropriate aniline derivative. ¹H NMR (d₆-DMSO) δ 10.14 (br s, 1H), 9.93 (s, 1H), 9.34 (br s, 1H), 9.06 (s, 1H), 8.33 (d, 1H), 8.02 (br s, 2H), 7.74 (d, 1H), 7.62 (br s, 1H), 7.42 (d, 2H), 7.37 (t, 2H), 7.11 (t, 1H), 7.05 (t, 1H), 2.95 (s, 3H); LCMS method A, (ES+) 458, RT=2.39 min.

### Example 204

### N-(2-(5-chloro-2-(4-cyanophenylamino)pyrimidin-4-ylamino)phenyl)methanesulfonamide

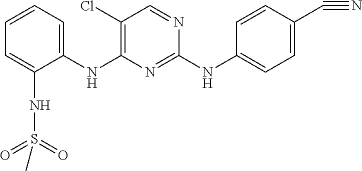

Synthesized according to the procedure in Example 1 using Intermediate 1h and the appropriate aniline derivative. ¹H NMR (d₆-DMSO) δ 10.24 (br s, 1H), 9.36 (s, 1H), 9.07 (br s, 1H), 8.32 (s, 1H), 7.74 (d, 1H), 7.66 (d, 2H), 7.52 (d, 2H), 7.48 (d, 1H), 7.38 (t, 2H), 2.94 (s, 3H); LCMS method A, (ES+) 414, RT=2.45 min.

### Example 205

### N-(2-(5-chloro-2-(3-cyanophenylamino)pyrimidin-4-ylamino)phenyl)methanesulfonamide

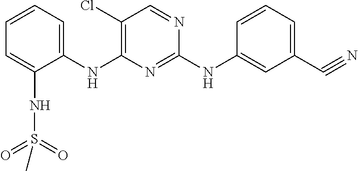

Synthesized according to the procedure in Example 1 using Intermediate 1h and the appropriate aniline derivative. ¹H NMR (d₆-DMSO) δ 10.15 (br s, 1H), 9.36 (s, 1H), 9.11 (br s, 1H), 8.33 (s, 1H), 7.93 (s, 1H), 7.76 (d, 1H), 7.69 (d, 1H), 7.48 (d, 1H), 7.36-7.48 (m, 4H), 2.95 (s, 3H); LCMS method A, (ES+) 414, RT=2.44 min.

### Example 206

### N-(2-(2-(3-(1H-pyrazol-1-yl)phenylamino)-5-chloropyrimidin-4-ylamino)phenyl)methanesulfonamide

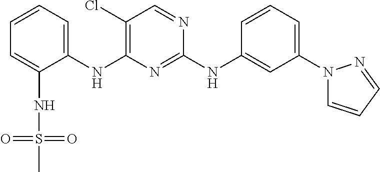

Synthesized according to the procedure in Example 1 using Intermediate 1h and the appropriate aniline derivative. ¹H NMR (d₆-DMSO) δ 10.27 (br s, 1H), 9.33 (s, 1H), 9.17 (br s, 1H), 8.33 (s, 1H), 8.25 (s, 1H), 7.93 (s, 1H), 7.81 (d, 1H), 7.73 (s, 1H), 7.42 (t, 3H), 7.24 (t, 2H), 7.12 (t, 1H), 6.54 (d 1H), 2.96 (s, 3H); LCMS method A, (ES+) 456, RT=2.39 min.

### Example 207

### N-(2-(2-(4-(1H-pyrazol-1-yl)phenylamino)-5-chloropyrimidin-4-ylamino)phenyl)methanesulfonamide

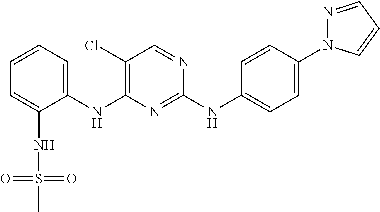

Synthesized according to the procedure in Example 1 using Intermediate 1h and the appropriate aniline derivative. ¹H NMR (d₆-DMSO) δ 10.51 (br s, 1H), 9.63 (br s, 1H), 9.37 (s, 1H), 8.42 (d, 1H), 7.72 (d, 1H), 7.68 (d, 1H), 7.58 (d, 2H), 7.54 (d, 1H), 7.45 (d, 2H), 7.39 (t, 2H), 6.52 (t, 1H), 2.95 (s, 3H); LCMS method A, (ES+) 456, RT=2.38 min.

### Example 208

### N-(2-(5-chloro-2-(3-(5-methyl-1,2,4-oxadiazol-3-yl)phenylamino)pyrimidin-4-ylamino)phenyl)methanesulfonamide

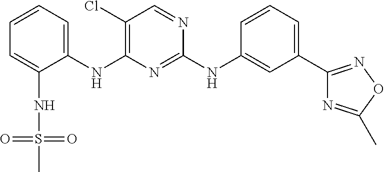

CZC00054068:001; JH360-069-7

Synthesized according to the procedure in Example 1 using Intermediate 1h and the appropriate aniline derivative. ¹H NMR (d₆-DMSO) δ 10.55 (br s, 1H), 9.54 (br s, 1H), 9.37 (s, 1H), 8.40 (d, 1H), 7.97 (s, 1H), 7.69 (d, 1H), 7.65 (d, 1H), 7.58 (d, 1H), 7.48 (d, 1H), 7.21-7.30 (m, 3H), 2.94 (s, 3H), 2.66 (s, 3H); LCMS method A, (ES+) 472, RT=2.35 min.

### Example 209

### N-(2-(5-chloro-2-(3-(3,5-dimethyl-M-pyrazol-1-yl)phenylamino)pyrimidin-4-ylamino)phenyl)methanesulfonamide

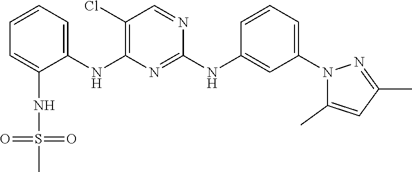

Synthesized according to the procedure in Example 1 using Intermediate 1h and the appropriate aniline derivative. ¹H NMR (d₆-DMSO) δ 10.81 (br s, 1H), 9.86 (br s, 1H), 9.38 (s, 1H), 8.44 (s, 1H), 7.61 (d, 1H), 7.53 (d, 1H), 7.36-7.44 (m, 4H), 7.23 (d, 2H), 6.10 (s, 1H), 2.94 (s, 3H), 2.23 (s, 3H), 2.19 (s, 3H); LCMS method A, (ES+) 483, RT=2.21 min.

### Example 210

### N-(2-(5-chloro-2-(4-(3,5-dimethyl-1H-pyrazol-1-yl)phenylamino)pyrimidin-4-ylamino)phenyl)methanesulfonamide

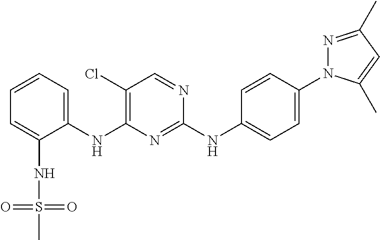

Synthesized according to the procedure in Example 1 using Intermediate 1h and the appropriate aniline derivative. ¹H NMR (d₆-DMSO) δ 10.73 (br s, 1H), 9.71 (br s, 1H), 9.34 (s, 1H), 8.42 (s, 1H), 7.58 (d, 1H), 7.49 (d, 1H), 7.37 (t, 4H), 7.23 (t, 1H), 7.16 (t, 1H), 7.10 (d, 2H), 6.12 (s, 1H), 2.92 (s, 3H), 2.20 (s, 3H), 2.19 (s, 3H); LCMS method A, (ES+) 483, RT=2.24 min.

### Example 211

### N-(2-(2-(3-(1H-1,2,4-triazol-1-yl)phenylamino)-5-chloropyrimidin-4-ylamino)phenyl)methanesulfonamide

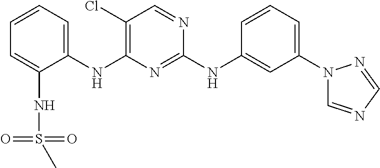

Synthesized according to the procedure in Example 1 using Intermediate 1h and the appropriate aniline derivative. ¹H NMR (d₆-DMSO) δ 9.64 (s, 1H), 9.29 (br s, 1H), 9.06 (s, 1H), 8.60 (s, 1H), 8.24 (s, 1H), 8.19 (s, 1H), 8.07 (s, 1H), 7.92 (d, 2H), 7.59 (d, 2H), 7.31-7.40 (m, 3H), 7.12-7.16 (m, 2H), 2.96 (s, 3H); LCMS method A, (ES+) 457, RT=2.27 min.

### Example 212

### N-(2-(2-(4-(1H-1,2,4-triazol-1-yl)phenylamino)-5-chloropyrimidin-4-ylamino)phenyl)methanesulfonamide

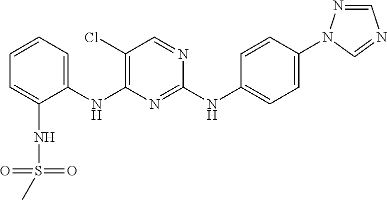

Synthesized according to the procedure in Example 1 using Intermediate 1h and the appropriate aniline derivative. ¹H NMR (d₆-DMSO) δ 9.58 (s, 1H), 9.26 (br s, 1H), 9.12 (s, 1H), 8.58 (s, 1H), 8.19 (s, 1H), 8.14 (s, 1H), 7.88 (d, 1H), 7.69 (d, 2H), 7.45 (d, 2H), 7.28-7.43 (m, 3H), 2.92 (s, 3H); LCMS method A, (ES+) 457, RT=2.25 min.

### Example 213

### N-(2-(5-chloro-2-(3-(3-methyl-1H-1,2,4-triazol-5-yl)phenylamino)pyrimidin-4-ylamino)phenyl)methanesulfonamide

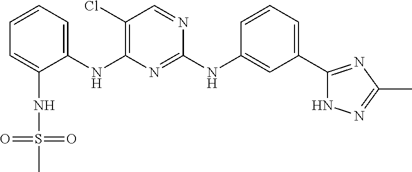

Synthesized according to the procedure in Example 1 using Intermediate 1h and the appropriate aniline derivative. ¹H NMR (d₆-DMSO) δ 9.56 (br s, 1H), 8.46 (br s, 1H), 8.22 (s, 1H), 8.16 (s, 1H), 7.92 (d, 1H), 7.73 (d, 2H), 7.63 (d, 2H), 7.44 (d, 1H), 7.28-7.32 (m, 2H), 2.93 (s, 3H), 2.36 (s, 1H); LCMS method A, (ES+) 470, RT=2.20 min.

### Example 214

### N-(2-(2-(4-(1H-tetrazol-1-yl)phenylamino)-5-chloropyrimidin-4-ylamino)phenyl)methanesulfonamide

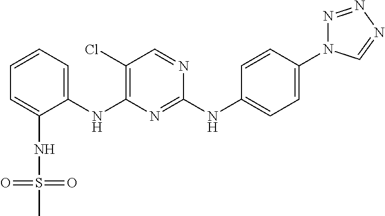

Synthesized according to the procedure in Example 1 using Intermediate 1h and the appropriate aniline derivative. ¹H NMR (d₆-DMSO) δ 10.56 (br s, 1H), 10.04 (s, 1H), 9.50 (br s, 1H), 9.38 (s, 1H), 8.39 (s, 1H), 7.72 (t, 1H), 7.64 (d, 4H), 7.53 (t, 1H), 7.39-7.42 (m, 2H), 2.95 (s, 3H); LCMS method A, (ES+) 458, RT=2.29 min.

### Example 215

### N-(2-(5-bromo-2-(4-(piperidin-1-yl)phenylamino)pyrimidin-4-ylamino)phenyl)methanesulfonamide

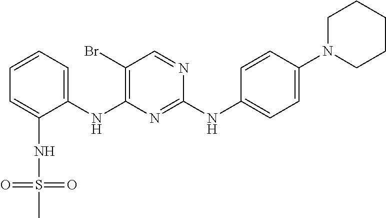

Synthesized according to the procedure in Example 1 using Intermediate 1c and the appropriate aniline derivative. ¹H NMR (d₆-DMSO) δ 9.13 (br s, 1H), 8.41 (br s, 1H), 8.19 (br s, 1H), 8.15 (br s, 1H), 8.05 (br s, 1H), 7.30-7.39 (m, 4H), 6.77 (d, 2H), 3.03 (t, 4H), 3.60 (s, 3H), 1.60-1.64 (m, 4H), 1.48-1.51 (m, 2H); LCMS method A, (ES+) 518, RT=2.24 min.

### Example 216

### N-(2-(2-(3-(1H-tetrazol-1-yl)phenylamino)-5-bromopyrimidin-4-ylamino)phenyl)methanesulfonamide

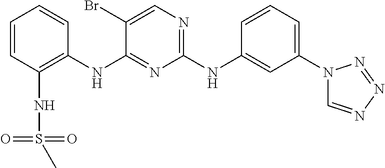

Synthesized according to the procedure in Example 1 using Intermediate 1c and the appropriate aniline derivative. ¹H NMR (d₆-DMSO) δ 9.89 (br s, 1H), 9.88 (s, 1H), 9.34 (br s, 1H), 8.67 (s, 1H), 8.33 (s, 1H), 8.04 (s, 1H), 7.86 (d, 1H), 7.62 (d, 1H), 7.36-7.39 (m, 2H), 7.07-7.10 (m, 2H), 2.95 (s, 3H); LCMS method A, (ES+) 504, RT=2.45 min.

### Example 217

### Determination of the Effect of the Compounds According to the Invention on ZAP-70

The compounds of the present invention as described in the previous examples can be tested in the ZAP-70 kinobeads assay as described (EP-A 1862802 and WO-A 2007/137867). Briefly, test compounds (at various concentrations) and the affinity matrix with the immobilized aminopyrido-pyrimidine ligand 24 are added to cell lysate aliquots and allowed to bind to the proteins in the lysate sample. After the incubation time the beads with captured proteins are separated from the lysate. Bound proteins are then eluted and the presence ZAP-70 is detected and quantified using a specific antibody in a dot blot procedure and the Odyssey infrared detection system. Conventionally, ZAP-70 kinase activity can be measured using purified or recombinant enzyme in a solution-based assay with protein or peptide substrates (Isakov et al., 1996, J. Biol. Chem. 271(26), 15753-15761; Moffat et al., 1999, Bioorg. Med. Chem. Letters 9, 3351-3356).

In general, compounds of the invention are effective for the inhibition of ZAP-70, with an IC₅₀ of <10 μM.

By this method (ZAP-70 kinobeads assay) the following compounds demonstrated an IC₅₀ between 1 and 10 μM: Examples 2, 4, 6, 10, 12, 13 15, 17, 18, 19, 20, 21, 24, 26, 27, 34, 38, 47, 49, 50, 52, 60, 61, 62, 65, 68, 72, 80, 81, 83, 85, 108, 121, 122, 144, 155, 157, 174, 186, 188, 189, 190, 191, 192, 194, 195, 196, 197, 200, 201, 202, 204, 209.

In addition, the following compounds demonstrated an IC₅₀ between 0.1 and 1 μM: Examples 1, 3, 7, 14, 16, 23, 25, 32, 33, 35, 36, 37, 64, 66, 67, 69, 71, 73, 74, 76, 77, 78, 82, 84, 86, 87, 88, 89, 90, 91, 92, 93, 94, 96, 97, 99, 100, 101, 102, 103, 104, 105, 106, 109, 110, 111, 115, 116, 119, 120, 130, 132, 139, 143, 149, 150, 154, 156, 158, 159, 161, 162, 163, 165, 168, 170, 171, 172, 176, 177, 181, 183, 184, 185, 187, 193, 198, 199, 203, 205, 206, 207, 208, 210, 211, 212, 213, 214, 215, 216.

In addition, the following compounds demonstrated an IC₅₀ below 0.1 μM: Examples 70, 75, 79, 95, 98, 107, 112, 113, 114, 117, 118, 123, 124, 125, 126, 127, 128, 129, 131, 133, 134, 135, 136, 137, 138, 140, 141, 142, 145, 146, 147, 148, 151, 152, 153, 160, 164, 166, 167, 169, 173, 175, 178, 179, 180, 182.

### Example 218

### Measurement of Calcium Ion Release in Cells

Compounds of the present invention were tested in a Calcium release assay as described below.

### Assay Principle

The development of fluorescent probes makes it possible to measure the concentration of intracellular free Calcium ions in single living cells. Cells are first loaded with a cell-permeable Ca²⁺-sensitive dye, then the test compound is added. Finally, cells are activated through the T cell receptor with an anti-CD3 antibody shortly before data acquisition on the flow cytometer. The release of Ca²⁺ is measured as a function of time after cell stimulation. This protocol describes the flow cytometry method using the Fluo-3 and Fluo-4 dyes (Minta et al., 1989, J. Biol. Chem. 264(14):8171-8178) to measure the intracellular Ca²⁺ concentration in Jurkat cells (see also June et al., 1997, Measurement of intracellular calcium ions by flow cytometry. In: Current Protocols in Cytometry (1997) Unit 9.8.1-9.8.19, John Wiley & Sons, Inc.).

### Ca²⁺ Release Assay Protocol

In general, cells should be handled in the shortest time possible to assure their stability. Therefore the preparation of all materials in advance is highly recommended as well as using any incubation or centrifugation time to prepare the next steps (e.g. preparing compound dilution or starting the flow cytometer).

1) Prepare a work plan indicating the number of samples to be analyzed including controls.
2) Cell harvest: Centrifuge 50 to 100 ml of a Jurkat cell culture for 5 minutes at 1100 rpm. Discard the supernatant, pool the resuspended cell pellets in a single 50 ml Falcon tube, and fill up to 50 ml with PBS—CaCl₂ (without FCS). Centrifuge again sample again.
3) Resuspend cell pellet with PBS—CaCl₂ (without FCS) to achieve a concentration not lower than 10×106 cells/ml. Prepare an adequate dilution to evaluate the cell concentration and count the cells.
4) Separate the volume of cells needed to be loaded (for example 107 cells for 20 samples) in a 50 ml Falcon tube. Fill up with PBS—CaCl₂ to 900 μl for 107 cells.
5) Prepare in the dark a 1:1 mix of FLUO-4 (1 mM)+Pluronic F-127 (20%). 5 μl of Fluo-4 are needed per 107 cells.
6) Prepare a 1/10 dilution of this mix with PBS—CaCl₂ (in the dark; for example 5 μl Fluo-4+5 μl F-127 completed to 100 μl).
7) Add the dye solution to the cells. Do not load more than 30×106 cells per tube.
8) Incubate the sample for 30 minutes in a cell incubator (37° C., 5% CO₂). Mix from time to time.
9) During this time prepare the adequate test compound dilution in PBS—CaCl₂ (with 10% FCS). The solution should be 10 times more concentrated than the desired final concentration. Vortex each dilution. Prepare also the antibody dilutions: anti-CD3 (1/200) and GAM (1/25).
10) Label FACS tubes and distribute in the corresponding tube 30 μl of the compound dilution or only buffer for the control tubes.
11) After 30 minutes of incubation wash the cells twice in PBS—CaCl₂ (with 10% FCS). During the washing steps make sure that the flow cytometer is already “ON” in order to warm the laser.
12) Resuspend the cell pellet in PBS—CaCl₂ (with 10% FCS) at a concentration of 1.5×10⁶ cells/ml.
13) Distribute 300 μl of cell suspension into the FACS tubes and mix gently. The compound incubation is starting. Keep samples at room temperature in the dark.
14) Open the settings of the cytometer. It is advised to have a template ready which is used for all measurements. Settings of the machine should also be the same from one experiment to the other which permits to evaluate the reproducibility of the cell preparation.
15) In Setup modus control your cell preparation on the cytometer. Cells should fit in the pre-defined gates.
16) During the compound incubation time, check also that cells are responding to the anti-CD3 activation as expected. Set a timer at 8 sec. Add 6 μl of anti-CD3 dilution (0.2 μg/ml final) and mix gently. Add 1 μl of GAM dilution (0.75 μg/ml final) and mix gently. Incubate the samples in water bath at 37° C. while starting timer. After 8 seconds of incubation, acquire data at medium speed for 200 seconds. A clear increase of fluorescence should appear after 1 minute.
17) Make sure that the cells rest and equilibrate at room temperature 15 minutes before FACS data acquisition
18) Data acquisition: Samples to be compared should be measured consecutively.
19) For a better reproducibility, make sure that data for the loaded cells are acquired within 2 hours.
20) Analyze the data by using the FlowJo® software (Tree Star, Inc.).

### Cell Culture

The Jurkat cell line J77 was obtained from American Type Cell Collection (ATCC). Jurkat cells were maintained in RPMI 1640 medium (Gibco, ref. 21875-034) supplemented with heat-inactivated fetal calf serum (Gibco, ref. 10270-106. FCS is heat-inactivated by in water bath for 45 minutes at 56° C.).

### Reagents

Fluo-3, AM (Molecular Probes, F14218, supplied as 1 ml of ready made 1 mM solution in DMSO and stored in 5 μl or 7.5 μl aliquots at −20° C.).

Fluo-4, AM (Molecular Probes, F14217, supplied as 1 ml of ready made 1 mM solution in DMSO and stored in 5 μl or 7.5 μl aliquots at −20° C.).

Pluronic F-127 (Molecular Probes, P3000MP, supplied as 1 ml of ready made 20% solution in DMSO).

PBS—CaCl₂—MgCl₂ (Gibco, 14040-91).

Anti-CD3 antibody (Calbiochem, 217570, supplied at 1 mg/ml).

Goat anti-mouse IgG antibody (GAM; Sigma, M8890, supplied at 6 mg/ml).

### Equipment

Flow cytometer (Becton-Dickinson, FACSCalibur) and FlowJo® software (Tree Star, Inc.).

### Results

By this method the following compounds demonstrated an IC₅₀ between 0.1 and 1 μM: 1, 2, 3, 4, 7, 14, 16, 18, 21, 25, 32, 35, 36, 37, 38, 44, 49, 50, 58, 62, 64, 66, 67, 69, 70, 72, 76, 77, 86, 88, 89, 90, 91, 92, 93, 94, 95, 96, 98, 101, 104, 106, 112, 113, 114, 116, 117, 118, 125, 129, 134, 137, 142, 145, 161, 162, 165, 166, 168, 169, 170, 171, 172, 177, 178, 183, 184, 215.

In addition, the following compounds demonstrated an IC₅₀ below 0.1 μM: 78, 79, 87, 97, 105, 107, 123, 124, 126, 127, 128, 130, 132, 135, 136, 138, 139, 140, 141, 146, 147, 150, 151, 152, 154, 158, 160, 164, 167, 173, 175, 176, 182.

1. A compound of formula (I) 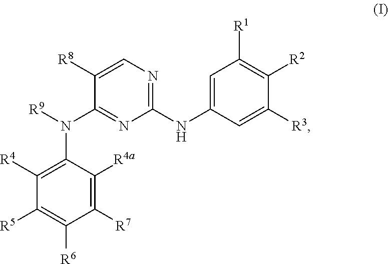
or a pharmaceutically acceptable salt, prodrug or metabolite thereof, wherein
R¹, R², R³ are independently selected from the group consisting of H; halogen; CN; C(O)OR¹⁰; OR¹⁰; C(O)R¹⁰; C(O)N(R¹⁰R^{10a}); S(O)₂N(R¹⁰R^{10a}); S(O)N(R¹⁰R^{10a}); S(O)₂R¹⁰; S(O)R¹⁰; N(R¹⁰)S(O)₂N(R^{10a}R^{10b}); SR¹⁰; N(R¹⁰R^{10a}); NO₂; OC(O)R¹⁰; N(R¹⁰)C(O)R^{10a}; N(R¹⁰)S(O)₂R^{10a}; N(R¹⁰)S(O)R^{10a}; N(R¹⁰)C(O)N(R^{10a}R^{10b}); N(R¹⁰)C(O)OR^{10a}; OC(O)N(R¹⁰R^{10a}); C₁₋₆ alkyl; C₂₋₆ alkenyl; C₂₋₆ alkynyl; and T, wherein C₁₋₆ alkyl; C₂₋₆ alkenyl; and C₂₋₆ alkynyl are optionally substituted with one or more R¹¹, which are the same or different;
Optionally, one of the pairs R¹/R² and R²/R³ is joined together with the phenyl ring to which it is attached to form a bicyclic ring T¹;
R¹⁰, R^{10a}, R^{10b} are independently selected from the group consisting of H; T; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹², which are the same or different;
R¹¹, R¹² are independently selected from the group consisting of T; halogen; CN; C(O)OR¹³; OR¹³; C(O)R¹³; C(O)N(R¹³R^{13a}); S(O)₂N(R¹³R^{13a}); S(O)N(R¹³R^{13a}); S(O)₂R¹³; S(O)R¹³; N(R¹³)S(O)₂N(R^{13a}R^{13b}); N(R¹³)S(O)N(R^{13a}R^{13b}); SR¹³; N(R¹³R^{13a}); NO₂; OC(O)R¹³; N(R¹³)C(O)R^{13a}; N(R¹³)S(O)₂R^{13a}; N(R¹³)S(O)R^{13a}; N(R¹³)C(O)N(R^{13a}R^{13b}); N(R¹³)C(O)OR^{13a}; OC(O)N(R¹³R^{13a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R¹³, R^{13a}, R^{13b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T is phenyl; C₃₋₇ cycloalkyl; or 4 to 7 membered heterocyclyl, wherein T is optionally substituted with one or more R¹⁴, which are the same or different;
T¹ is naphthyl; indenyl; indanyl; or 9 to 11 membered benzo-fused heterobicyclyl, wherein T¹ is optionally substituted with one or more R¹⁵, which are the same or different;
R¹⁴, R¹⁵ are independently selected from the group consisting of halogen; CN; C(O)OR¹⁶; OR¹⁶; oxo (═O), where the ring is at least partially saturated; C(O)R¹⁶; C(O)N(R¹⁶R^{16a}); S(O)₂N(R¹⁶R^{16a}); S(O)N(R¹⁶R^{16a}); S(O)₂R¹⁶; S(O)R¹⁶; N(R¹⁶)S(O)₂N(R^{16a}R^{16b}); N(R¹⁶)S(O)N(R^{16a}R^{16b}); SR¹⁶; N(R¹⁶R^{16a}); NO₂; OC(O)R¹⁶; N(R¹⁶)C(O)R^{16a}; N(R¹⁶)S(O)₂R^{16a}; N(R¹⁶)S(O)R^{16a}; N(R¹⁶)C(O)N(R^{16a}R^{16b}); N(R¹⁶)C(O)OR^{16a}; OC(O)N(R¹⁶R^{16a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R¹⁶, R^{16a}, R^{16b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R⁴, R⁵, R⁶, R⁷, R^{4a} are independently selected from the group consisting of H; X¹; halogen; CN; C(O)OR¹⁷; OR¹⁷; C(O)R¹⁷; C(O)N(R¹⁷R^{17a}); S(O)₂N(R¹⁷R^{17a}); S(O)N(R¹⁷R^{17a}); S(O)₂R¹⁷; S(O)R¹⁷; SR¹⁷; N(R¹⁷R^{17a}); NO₂; OC(O)R¹⁷; N(R¹⁷)C(O)R^{17a}; N(R¹⁷)S(O)₂R^{17a}; N(R¹⁷)S(O)R^{17a}; N(R¹⁷)C(O)N(R^{17a}R^{17b}); N(R¹⁷)C(O)OR^{17a}; OC(O)N(R¹⁷R^{17a}); C₁₋₆ alkyl; C₂₋₆ alkenyl; C₂₋₆ alkynyl; and T², wherein C₁₋₆ alkyl; C₂₋₆ alkenyl; and C₂₋₆ alkynyl are optionally substituted with one or more R¹⁸, which are the same or different and
wherein one of R⁴, R⁵, R⁶, R⁷, R^{4a} is X¹;
Optionally, one of the pairs R⁴/R⁵, R⁵/R⁶, R⁶/R⁷, R⁷/R^{4a} is joined together with the phenyl ring to which it is attached to form a bicyclic ring T³;
R¹⁷, R^{17a}, R^{17b} are independently selected from the group consisting of H; T²; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more R¹⁹, which are the same or different;
R¹⁸, R¹⁹ are independently selected from the group consisting of T²; halogen; CN; C(O)OR²⁰; OR²⁰; C(O)R²⁰; C(O)N(R²⁰R^{20a}); S(O)₂N(R²⁰R^{20a}); S(O)N(R²⁰R^{20a}); S(O)₂R²⁰; S(O)R²⁰; N(R²⁰)S(O)₂N(R^{20a}R^{20b}); N(R²⁰)S(O)N(R^{20a}R^{20b}); SR²⁰; N(R²⁰R^{20a}); NO₂; OC(O)R²⁰; N(R²⁰)C(O)R^{20a}; N(R²⁰)S(O)₂R^{20a}; N(R²⁰)S(O)R^{20a}; N(R²⁰)C(O)N(R^{20a}R^{20b}); N(R²⁰)C(O)OR^{20a}; OC(O)N(R²⁰R^{20a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R²⁰; R^{20a}; R^{20b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T² is phenyl; C₃₋₇ cycloalkyl; or 4 to 7 membered heterocyclyl, wherein T² is optionally substituted with one or more R²¹, which are the same or different;
T³ is naphthyl; indenyl; indanyl; or 9 to 11 membered benzo-fused heterobicyclyl, wherein T³ is optionally substituted with one or more R²², which are the same or different;
R²¹, R²² are independently selected from the group consisting of halogen; CN; C(O)OR²³; OR²³; oxo (═O), where the ring is at least partially saturated; C(O)R²³; C(O)N(R²³R^{23a}); S(O)₂N(R²³R^{23a}); S(O)N(R²³R^{23a}); S(O)₂R²³; S(O)R²³; N(R²³)S(O)₂N(R^{23a}R^{23b}); N(R²³)S(O)N(R^{23a}R^{23b}); SR²³; N(R²³R^{23a}); NO₂; OC(O)R²³; N(R²³)C(O)R^{23a}; N(R²³)S(O)₂R^{23a}; N(R²³)S(O)R^{23a}; N(R²³)C(O)N(R^{23a}R^{23b}); N(R²³)C(O)OR^{23a}; OC(O)N(R²³R^{23a}); C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R²³, R^{23a}, R^{23b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
X¹ is N(R^{24a})S(O)₂R²⁴;
R⁹, R^{24a} are independently selected from the group consisting of H; C₁₋₄ alkyl; C₃₋₅ cycloalkyl; and C₃₋₅ cycloalkylmethyl, wherein C₁₋₄ alkyl; C₃₋₅ cycloalkyl and C₃₋₅ cycloalkylmethyl are optionally substituted with one or more halogen, which are the same or different;
R²⁴ is T⁴; C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more R²⁵, which are the same or different;
R²⁵ is T⁴; halogen; CN; C(O)OR²⁶; OR²⁶; C(O)R²⁶; C(O)N(R²⁶R^{26a}); S(O)₂N(R²⁶R^{26a}); S(O)N(R²⁶R^{26a}); S(O)₂R²⁶; S(O)R²⁶; N(R²⁶)S(O)₂N(R^{26a}R^{26b}); N(R²⁶)S(O)N(R^{26a}R²⁶); SR²⁶; N(R²⁶R^{26a}); NO₂; OC(O)R²⁶; N(R²⁶)C(O)R^{26a}; N(R²⁶)S(O)₂R^{26a}; N(R²⁶)S(O)R^{26a}; N(R²⁶)C(O)N(R^{26a}R^{26b}); N(R²⁶)C(O)OR^{26a}; OC(O)N(R²⁶R^{26a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more halogen, which are the same or different;
R²⁶, R^{26a}, R^{26b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
T⁴ is phenyl; C₃₋₇ cycloalkyl; or 4 to 7 membered heterocyclyl, wherein T⁴ is optionally substituted with one or more R²⁷, which are the same or different;
R²⁷ is halogen; CN; C(O)OR²⁸; OR²⁸; oxo (═O), where the ring is at least partially saturated; C(O)R²⁸; C(O)N(R²⁸R^{28a}); S(O)₂N(R²⁸R^{28a}); S(O)N(R²⁸R^{28a}); S(O)₂R²⁸; S(O)R²⁸; N(R²⁸)S(O)₂N(R^{28a}R²⁸); N(R²⁸)S(O)N(R^{28a}R²⁸); SR²⁸; N(R²⁸R^{28a}); NO₂; OC(O)R²⁸; N(R²⁸)C(O)R^{28a}; N(R²⁸)S(O)₂R^{28a}; N(R²⁸)S(O)R^{28a}; N(R²⁸)C(O)N(R^{28a}R^{28b}); N(R²⁸)C(O)OR^{28a}; OC(O)N(R²⁸R^{28a}); C₁₋₆ alkyl; C₂₋₆ alkenyl; or C₂₋₆ alkynyl, wherein C₁₋₆ alkyl; C₂₋₆ alkenyl; and C₂₋₆ alkynyl are optionally substituted with one or more halogen, which are the same or different;
R²⁸, R^{28a}, R^{28b} are independently selected from the group consisting of H; C₁₋₆ alkyl; C₂₋₆ alkenyl; and C₂₋₆ alkynyl, wherein C₁₋₆ alkyl; C₂₋₆ alkenyl; and C₂₋₆ alkynyl are optionally substituted with one or more halogen, which are the same or different;
R⁸ is H; F; Cl; Br; CN; C₁₋₄ alkyl; CH₂F; CHF₂; CF₃; OH; OCH₃; NO₂; NH₂; NHCH₃; N(CH₃)₂; or NO₂.

2. A compound of claim 1, wherein R^{4a} is X¹.

3. A compound of claim 1, wherein none of the pairs R¹/R² and R²/R³ is joined together with the phenyl ring to which it is attached to form a bicyclic ring T¹.

4. A compound of claim 1, wherein R¹, R², R³ are independently selected from the group consisting of H; halogen; CN; OR¹⁰; NO₂; C(O)R¹⁰; SR¹⁰; N(R¹⁰R^{10a}); T; and C₁₋₄ alkyl, wherein C₁₋₄ alkyl is optionally substituted with one or more halogen, which are the same or different.

5. A compound of claim 1, wherein R¹⁰, R^{10a} are independently selected from the group consisting of H; and C₁₋₄ alkyl, wherein C₁₋₄ alkyl is optionally substituted with one or more halogen, which are the same or different.

6. A compound of claim 1, wherein R¹, R², R³ are independently selected from the group consisting of H; F; Cl; CN; OH; OCH₃; OCH₂CH₃; OCH₂F; OCHF₂; OCF₃; OCH₂CH₂F; OCH₂CHF₂; OCH₂CF₃; OCHFCH₂F; OCHFCHF₂; OCHFCF₃; OCF₂CH₂F; OCF₂CHF₂; OCF₂CF₃; NO₂; C(O)CH₃; SH; SCH₃; SCH₂F; SCHF₂; SCF₃; NH₂; NHCH₃; N(CH₃)₂; CH₃; CH₂CH₃; CH₂F; CHF₂; CF₃; CH₂CH₂F; CH₂CHF₂; CH₂CF₃; CHFCH₂F; CHFCHF₂; CHFCF₃; CF₂CH₂F; CF₂CHF₂; and CF₂CF₃.

7. A compound of claim 1, wherein T is 4 to 7 membered heterocyclyl.

8. A compound of claim 1, wherein T is 5 or 6 membered heterocyclyl.

9. A compound of claim 1, wherein T is imidazolyl; oxazolyl; thiazolyl; pyrazolyl; tetrazolyl; triazolyl; oxadiazolyl; morpholinyl; piperazinyl; pyrrolyl; pyrrolidinyl; or piperidinyl.

10. A compound of claim 1, wherein R¹, R² are joined together with the phenyl ring to which they are attached to form 9 to 11 membered benzo-fused heterobicyclyl.

11. A compound of claim 10, wherein the bicyclic ring is benzodioxane; benzothiazole; benzomorpholine; indole; indoline; indazole; benzoxazole; benzothiazole; or benzotriazole.

12. A compound of claim 1, wherein each R¹⁵ is independently selected from the group consisting of F; Cl; oxo (═O), where the ring is at least partially saturated; OH; OCH₃; OCH₂CH₃; OCH₂F; OCHF₂; OCF₃; OCH₂CH₂F; OCH₂CHF₂; OCH₂CF₃; OCHFCH₂F; OCHFCHF₂; OCHFCF₃; OCF₂CH₂F; OCF₂CHF₂; OCF₂CF₃; NO₂; C(O)CH₃; SH; SCH₃; SCH₂F; SCHF₂; SCF₃; NH₂; NHCH₃; N(CH₃)₂; CH₃; CH₂CH₃; CH₂F; CHF₂; CF₃; CH₂CH₂F; CH₂CHF₂; CH₂CF₃; CHFCH₂F; CHFCHF₂; CHFCF₃; CF₂CH₂F; CF₂CHF₂; and CF₂CF₃.

13. A compound of claim 1, wherein one of R⁴, R⁵, R⁶, R⁷, R^{4a} is X¹ and the others are selected from the group consisting of H; F; OH; OCH₃; OCH₂CH₃; OCH(CH₃)₂; CH₃; CH₂CH₃; and CH(CH₃)₂.

14. A compound of claim 1, wherein R⁹; and R^{24a} are independently selected from the group consisting of H; CH₃; and CH₂CH₃.

15. A compound of claim 1, wherein R²⁴ is C₁₋₄ alkyl.

16. A compound of claim 1, wherein R²⁴ is CH₃.

17. A compound of claim 1, wherein R²⁴ is T⁴; or C₁₋₄ alkyl, wherein C₁₋₄ alkyl is substituted with one or more R²⁵, which are the same or different.

18. A compound of claim 1, wherein T⁴ is phenyl; thiazolyl; imidazolyl; pyridyl; morpholinyl; piperazinyl, pyrrolidinyl; piperidinyl; or cyclopropyl.

19. A compound of claim 1, wherein R²⁵ is F; Cl; OH; OCH₃; OCH₂CH₃; OCH₂F; OCHF₂; OCF₃; OCH₂CH₂F; OCH₂CHF₂; OCH₂CF₃; OCHFCH₂F; OCHFCHF₂; OCHFCF₃; OCF₂CH₂F; OCF₂CHF₂; OCF₂CF₃; NO₂; C(O)CH₃; SH; SCH₃; SCH₂F; SCHF₂; SCF₃; NH₂; NHCH₃; and N(CH₃)₂.

20. A compound of claim 1, wherein R²⁴ is CH₂CF₃; T⁴; CH₂-T⁴; CH₂CH₂-T⁴; CH₂CH₂NHCH₃; or CH₂CH₂N(CH₃)₂.

21. A compound of claim 1, wherein R²⁷ is CH₃.

22. A compound of claim 1, wherein R⁸ is H; F; Cl; Br; CN; CH₃; CH(CH₃)₂; CH₂F; CHF₂; CF₃; OH; OCH₃; NO₂; NH₂; NHCH₃; N(CH₃)₂; or NO₂.

23. A compound of claim 1, wherein R⁸ is H; CH₃; Br; or F.

24. A compound of claim 1 selected from the group consisting of
N-(2-(5-fluoro-2-(3,4,5-trimethoxyphenylamino)pyrimidin-4-ylamino)phenyl)methanesulfonamide;
N-(2-(5-fluoro-2-(4-fluorophenylamino)pyrimidin-4-ylamino)phenyl)methanesulfonamide;
N-(2-(2-(3,5-dimethylphenylamino)-5-fluoropyrimidin-4-ylamino)phenyl)methanesulfonamide;
N-(2-(2-(2,3-dihydrobenzo[b][1,4]dioxin-6-ylamino)-5-fluoropyrimidin-4-ylamino)phenyl)methanesulfonamide;
N-(2-(2-(3,5-difluorophenylamino)-5-fluoropyrimidin-4-ylamino)phenyl)methanesulfonamide;
N-(2-(5-fluoro-2-(3-fluorophenylamino)pyrimidin-4-ylamino)phenyl)methanesulfonamide;
N-(2-(2-(4-(dimethylamino)phenylamino)-5-fluoropyrimidin-4-ylamino)phenyl)methane sulfonamide;
N-(2-(2-(3,5-bis(trifluoromethyl)phenylamino)-5-fluoropyrimidin-4-ylamino)phenyl)methane sulfonamide;
N-(2-(5-fluoro-2-(4-(trifluoromethyl)phenylamino)pyrimidin-4-ylamino)phenyl)methane sulfonamide;
N-(2-(2-(4-chloro-3-methoxyphenylamino)-5-fluoropyrimidin-4-ylamino)phenyl)methane sulfonamide;
N-(2-(5-fluoro-2-(4-methyl-3-nitrophenylamino)pyrimidin-4-ylamino)phenyl)methane sulfonamide;
N-(2-(2-(3-chlorophenylamino)-5-fluoropyrimidin-4-ylamino)phenyl)methane sulfonamide;
N-(2-(2-(3-ethoxyphenylamino)-5-fluoropyrimidin-4-ylamino)phenyl)methane sulfonamide;
N-(2-(2-(3-acetylphenylamino)-5-fluoropyrimidin-4-ylamino)phenyl)methane sulfonamide;
N-(2-(5-fluoro-2-(3-(methylthio)phenylamino)pyrimidin-4-ylamino)phenyl)methanesulfonamide;
N-(2-(2-(benzo[d]thiazol-5-ylamino)-5-fluoropyrimidin-4-ylamino)phenyl)methanesulfonamide;
N-(2-(2-(3-(1H-pyrazol-1-yl)phenylamino)-5-fluoropyrimidin-4-ylamino)phenyl)methanesulfonamide;
N-(2-(5-fluoro-2-(2-methylbenzo[d]thiazol-5-ylamino)pyrimidin-4-ylamino)phenyl)methanesulfonamide;
N-(2-(2-(3-chloro-4-methoxyphenylamino)-5-fluoropyrimidin-4-ylamino)phenyl)methanesulfonamide;
N-(2-(5-fluoro-2-(4-methyl-3-oxo-3,4-dihydro-2H-benzo[b][1,4]oxazin-6-ylamino)pyrimidin-4-ylamino)phenyl)methanesulfonamide;
N-(2-(2-(3-(1H-1,2,4-triazol-1-yl)phenylamino)-5-fluoropyrimidin-4-ylamino)phenyl)methanesulfonamide;
N-(2-(2-(3-(3,5-dimethyl-1H-pyrazol-1-yl)phenylamino)-5-fluoropyrimidin-4-ylamino)phenyl)methanesulfonamide;
N-(2-(5-fluoro-2-(3-oxo-3,4-dihydro-2H-benzo[b][1,4]oxazin-7-ylamino)pyrimidin-4-ylamino)phenyl)methanesulfonamide;
N-(2-(5-fluoro-2-(3-(5-methyl-4H-1,2,4-triazol-3-yl)phenylamino)pyrimidin-4-ylamino)phenyl)methanesulfonamide;
N-(2-(5-fluoro-2-(4-methyl-3-oxo-3,4-dihydro-2H-benzo[b][1,4]oxazin-7-ylamino)pyrimidin-4-ylamino)phenyl)methanesulfonamide;
N-(2-(2-(3-(difluoromethoxy)phenylamino)-5-fluoropyrimidin-4-ylamino)phenyl)methanesulfonamide;
N-(2-(2-(4-(difluoromethoxy)phenylamino)-5-fluoropyrimidin-4-ylamino)phenyl)methanesulfonamide;
N-(2-(5-fluoro-2-(4-(trifluoromethoxy)phenylamino)pyrimidin-4-ylamino)phenyl)methanesulfonamide;
N-(2-(5-fluoro-2-(3-(trifluoromethoxy)phenylamino)pyrimidin-4-ylamino)phenyl)methanesulfonamide;
N-(2-(2-(4-chlorophenylamino)-5-fluoropyrimidin-4-ylamino)phenyl)methanesulfonamide;
N-(2-(5-fluoro-2-(3-(1,1,2,2-tetrafluoroethoxy)phenylamino)pyrimidin-4-ylamino)phenyl)methanesulfonamide;
N-(2-(2-(1H-indazol-6-ylamino)-5-fluoropyrimidin-4-ylamino)phenyl)methanesulfonamide;
N-(2-(2-(1H-benzo[d][1,2,3]triazol-6-ylamino)-5-fluoropyrimidin-4-ylamino)phenyl)methanesulfonamide;
N-(2-(5-fluoro-2-(phenylamino)pyrimidin-4-ylamino)phenyl)methanesulfonamide;
N-(2-(5-fluoro-2-(4-methoxyphenylamino)pyrimidin-4-ylamino)phenyl)methanesulfonamide;
N-(2-(2-(3,4-dimethoxyphenylamino)-5-fluoropyrimidin-4-ylamino)phenyl)methanesulfonamide;
N-(2-(2-(3,5-dimethoxyphenylamino)-5-fluoropyrimidin-4-ylamino)phenyl)methanesulfonamide;
N-(2-(5-fluoro-2-(3-methoxyphenylamino)pyrimidin-4-ylamino)phenyl)methanesulfonamide;
N-(4-(5-fluoro-2-(3,4,5-trimethoxyphenylamino)pyrimidin-4-ylamino)phenyl)thiophene-3-sulfonamide;
N-(4-(5-fluoro-2-(3,4,5-trimethoxyphenylamino)pyrimidin-4-ylamino)phenyl)thiophene-2-sulfonamide;
N-(4-(5-fluoro-2-(3,4,5-trimethoxyphenylamino)pyrimidin-4-ylamino)phenyl)pyridine-3-sulfonamide;
N-(4-(5-fluoro-2-(3,4,5-trimethoxyphenylamino)pyrimidin-4-ylamino)phenyl)-1-methyl-1H-imidazole-4-sulfonamide;
N-(4-(5-fluoro-2-(3,4,5-trimethoxyphenylamino)pyrimidin-4-ylamino)phenyl)-1-phenylmethanesulfonamide;
N-(4-(5-fluoro-2-(3,4,5-trimethoxyphenylamino)pyrimidin-4-ylamino)phenyl)benzenesulfonamide;
2,2,2-trifluoro-N-(4-(5-fluoro-2-(3,4,5-trimethoxyphenylamino)pyrimidin-4-ylamino)phenyl)ethanesulfonamide;
2,2,2-trifluoro-N-(2-(5-fluoro-2-(3,4,5-trimethoxyphenylamino)pyrimidin-4-ylamino)phenyl)ethanesulfonamide.
N-(2-(5-fluoro-2-(3,4,5-trimethoxyphenylamino)pyrimidin-4-ylamino)phenyl)benzenesulfonamide;
N-(2-(5-fluoro-2-(3,4,5-trimethoxyphenylamino)pyrimidin-4-ylamino)phenyl)-1-phenylmethanesulfonamide;
N-(2-(5-fluoro-2-(3,4,5-trimethoxyphenylamino)pyrimidin-4-ylamino)phenyl)thiopehene-3-sulfonamide;
N-(2-(5-fluoro-2-(3,4,5-trimethoxyphenylamino)pyrimidin-4-ylamino)phenyl)thiopehene-2-sulfonamide;
N-(2-(5-fluoro-2-(3,4,5-trimethoxyphenylamino)pyrimidin-4-ylamino)phenyl)-1-methyl-1H-imidazole-4-sulfonamide;
N-(2-(5-fluoro-2-(3,4,5-trimethoxyphenylamino)pyrimidin-4-ylamino)phenyl)pyridine-3-sulfonamide;
N-(2-(5-fluoro-2-(3,4,5-trimethoxyphenylamino)pyrimidin-4-ylamino)phenyl)pyridine-2-sulfonamide;
N-(3-(5-fluoro-2-(3,4,5-trimethoxyphenylamino)pyrimidin-4-ylamino)phenyl)thiophene-2-sulfonamide;
N-(3-(5-fluoro-2-(3,4,5-trimethoxyphenylamino)pyrimidin-4-ylamino)phenyl)thiophene-3-sulfonamide;
N-(3-(5-fluoro-2-(3,4,5-trimethoxyphenylamino)pyrimidin-4-ylamino)phenyl)-1-methyl-1H-imidazole-4-sulfonamide;
N-(3-(5-fluoro-2-(3,4,5-trimethoxyphenylamino)pyrimidin-4-ylamino)phenyl)pyridine-2-sulfonamide;
N-(3-(5-fluoro-2-(3,4,5-trimethoxyphenylamino)pyrimidin-4-ylamino)phenyl)pyridine-3-sulfonamide;
N-(3-(5-fluoro-2-(3,4,5-trimethoxyphenylamino)pyrimidin-4-ylamino)phenyl)benzenesulfonamide hydrochloride;
2,2,2-trifluoro-N-(3-(5-fluoro-2-(3,4,5-trimethoxyphenylamino)pyrimidin-4-ylamino)phenyl)ethanesulfonamide;
2-(Dimethylamino)-N-(2-(5-fluoro-2-(3,4,5-trimethoxyphenylamino)pyrimidin-4-ylamino)phenyl)ethanesulfonamide;
N-(2-(5-fluoro-2-(3,4,5-trimethoxyphenylamino)pyrimidin-4-ylamino)phenyl)-2-(methylamino)ethanesulfonamide;
N-(2-(5-fluoro-2-(3,4,5-trimethoxyphenylamino)pyrimidin-4-ylamino)-2-morpholinoethanesulfonamide;
N-(3-(5-fluoro-2-(3,4,5-trimethoxyphenylamino)pyrimidin-4-ylamino)phenyl)-2-(methylamino)ethanesulfonamide;
2-(Dimethylamino)-N-(3-(5-fluoro-2-(3,4,5-trimethoxyphenylamino)pyrimidin-4-ylamino)phenyl)ethanesulfonamide;
N-(2-(5-methyl-2-(3,4,5-trimethoxyphenylamino)pyrimidin-4-ylamino)phenyl)methanesulfonamide;
N-(2-(2-(3,5-dimethylphenylamino)-5-methylpyrimidin-4-ylamino)phenyl)methanesulfonamide;
N-(2-(5-nitro-2-(3,4,5-trimethoxyphenylamino)pyrimidin-4-ylamino)phenyl)methanesulfonamide;
N-(2-(2-(3,4,5-trimethoxyphenylamino)pyrimidin-4-ylamino)phenyl)methanesulfonamide;
N-(2-(5-bromo-2-(3,4,5-trimethoxyphenylamino)pyrimidin-4-ylamino)phenyl)methanesulfonamide;
N-(2-(5-fluoro-2-(3,4,5-trimethoxyphenylamino)pyrimidin-4-ylamino)phenyl)-N-methylmethanesulfonamide;
N-(2-(2-(3,5-dimethylphenylamino)-5-fluoropyrimidin-4-ylamino)phenyl)-N-methylmethanesulfonamide;
N-(2-(5-fluoro-2-(3-methoxy-4-methylphenylamino)pyrimidin-4-ylamino)phenyl)methanesulfonamide;
N-(2-(2-(3,4-dimethoxy-5-(2-(pyrrolidin-1-yl)ethoxy)phenylamino)-5-fluoropyrimidin-4-ylamino)phenyl)methanesulfonamide;
N-(2-(2-(3,5-dimethoxy-4-(2-(pyrrolidin-1-yl)ethoxy)phenylamino)-5-fluoropyrimidin-4-ylamino)phenyl)methanesulfonamide;
N-(2-(5-fluoro-2-(3-hydroxyphenylamino)pyrimidin-4-ylamino)phenyl)methanesulfonamide;
N-(2-(5-fluoro-2-(3-(2-morpholinoethoxy)phenylamino)pyrimidin-4-ylamino)phenyl)methanesulfonamide;
N-(2-(5-fluoro-2-(3-(2-hydroxyethoxy)phenylamino)pyrimidin-4-ylamino)phenyl)methanesulfonamide;
N-(2-(5-fluoro-2-(3-(2-(piperidin-1-yl)ethoxy)phenylamino)pyrimidin-4-ylamino)phenyl)methanesulfonamide formate;
3-(5-fluoro-4-(2-(methylsulfonamido)phenylamino)pyrimidin-2-ylamino)-N-methylbenzamide;
N,N-diethyl-3-(5-fluoro-4-(2-(methylsulfonamido)phenylamino)pyrimidin-2-ylamino)benzamide;
N-(2-(5-fluoro-2-(3-(pyrrolidine-1-carbonyl)phenylamino)pyrimidin-4-ylamino)phenyl)methanesulfonamide;
N-cyclopropyl-3-(5-fluoro-4-(2-(methylsulfonamido)phenylamino)pyrimidin-2-ylamino)benzamide;
3-(5-fluoro-4-(2-(methylsulfonamido)phenylamino)pyrimidin-2-ylamino)-N,N-dimethylbenzamide;
N-(2-(5-fluoro-2-(3-(pyrrolidin-1-yl)phenylamino)pyrimidin-4-ylamino)phenyl)methanesulfonamide;
N-(2-(5-fluoro-2-(4-(2-morholinoethoxy)phenylamino)pyrimidin-4-ylamino)phenyl)methanesulfonamide;
N-(2-(5-fluoro-2-(4-(piperidin-1-yl)ethoxy)phenylamino)pyrimidin-4-ylamino)phenyl)methanesulfonamide;
N-(2-(5-fluoro-2-(3-methoxy-4-(pyrrolidin-1-yl)phenylamino)pyrimidin-4-ylamino)phenyl)methanesulfonamide;
N-(2-(5-Fluoro-2-(4-(2-(4-methylpiperazin-1-yl)ethoxy)phenylamino)pyrimidin-4-ylamino)phenyl)methanesulfonamide;
N-(2-(5-fluoro-2-(4-(3-piperidin-1-yl)propoxy)phenylamino)pyrimidin-4-ylamino)phenyl)methanesulfonamide;
N-(2-(5-fluoro-2-(4-(3-(4-methylpiperazin-1-yl)propoxy)phenylamino)pyrimidin-4-ylamino)phenyl)methanesulfonamide;
N-(2-(5-fluoro-2-(4-(3-pyrrolidin-1-yl)propoxy)phenylamino)pyrimidin-4-ylamino)phenyl)methanesulfonamide;
N-(2-(5-fluoro-2-(4-(2-pyrrolidin-1-yl)ethylamino)phenylamino)pyrimidin-4-ylamino)phenyl)methanesulfonamide formate;
N-(2-(5-fluoro-2-(3-methoxy-5-(2-(pyrrolidin-1-yl)ethoxy)phenylamino)pyrimidin-4-ylamino)phenyl)methanesulfonamide formate salt;
N-(2-(5-fluoro-2-(3-(2-(pyrrolidin-1-yl)ethoxy)phenylamino)pyrimidin-4-ylamino)phenyl)methanesulfonamide;
N-(2-(5-fluoro-2-(4-(2-(pyrrolidin-1-yl)ethoxy)phenylamino)pyrimidin-4-ylamino)phenyl)methanesulfonamide;
N-(2-(5-fluoro-2-(3-((1-methylpiperidin-2-yl)methoxy)phenylamino)pyrimidin-4-ylamino)phenyl)methanesulfonamide;
N-(2-(5-fluoro-2-(3-((1-methylpiperidin-3-yl)methoxy)phenylamino)pyrimidin-4-ylamino)phenyl)methanesulfonamide;
2-(3-(5-fluoro-4-(2-(methylsulfonamido)phenylamino)pyrimidin-2-ylamino)phenoxy)acetic acid hydrochloride;
N,N-diethyl-2-(3-(5-fluoro-4-(2-(methylsulfonamido)phenylamino)pyrimidin-2-ylamino)phenoxy)acetamide 2,2,2-trifluoroacetate;
N-ethyl-2-(3-(5-fluoro-4-(2-(methylsulfonamido)phenylamino)pyrimidin-2-ylamino)phenoxy)acetamide;
N-(2-(5-bromo-2-(phenylamino)pyrimidin-4-ylamino)phenyl)methanesulfonamide hydrochloride;
N-(2-(5-bromo-2-(3-(difluoromethoxy)phenylamino)pyrimidin-4-ylamino)phenyl)methanesulfonamide;
N-(2-(5-bromo-2-(3-methoxy-4-methylphenylamino)pyrimidin-4-ylamino)phenyl)methanesulfonamide;
N-(2-(5-bromo-2-(3,5-dimethoxyphenylamino)pyrimidin-4-ylamino)phenyl)methanesulfonamide;
N-(2-(5-bromo-2-(4-methoxyphenylamino)pyrimidin-4-ylamino)phenyl)methanesulfonamide;
N-(2-(5-bromo-2-(3,4-dimethoxyphenylamino)pyrimidin-4-ylamino)phenyl)methanesulfonamide;
N-(2-(5-fluoro-2-(4-methoxyphenylamino)pyrimidin-4-ylamino)phenyl)-N-methylmethanesulfonamide;
N-(2-(2-(3,4-dimethoxyphenylamino)-5-fluoropyrimidin-4-ylamino)phenyl)-N-methylmethanesulfonamide;
N-(2-(5-fluoro-2-(4-(2-(piperidin-1-yl)ethoxy)phenylamino)pyrimidin-4-ylamino)phenyl)-N-methylmethanesulfonamide;
N-(2-(5-fluoro-2-(4-(2-morpholinoethoxy)phenylamino)pyrimidin-4-ylamino)phenyl)-N-methylmethanesulfonamide;
N-(2-(5-fluoro-2-(3-(2-(piperidin-1-yl)ethoxy)phenylamino)pyrimidin-4-ylamino)-6-methylphenyl)methanesulfonamide;
N-(2-(2-(3,5-dimethoxy-4-(2-(piperidin-1-yl)ethoxy)phenylamino)-5-fluoropyrimidin-4-ylamino)-6-methylphenyl)methanesulfonamide;
N-(2-(2-(3,4-dimethoxy-5-(2-(piperidin-1-yl)ethoxy)phenylamino)-5-fluoropyrimidin-4-ylamino)-6-methylphenyl)methanesulfonamide;
N-(2-(5-fluoro-2-(3-(2-methoxyethoxy)phenylamino)pyrimidin-4-ylamino)-6-methylphenyl)methanesulfonamide;
N-(2-(5-fluoro-2-(3,4,5-trimethoxyphenylamino)pyrimidin-4-ylamino)-6-methylphenyl)methanesulfonamide;
N-(2-(2-(3-ethoxy-4,5-dimethoxyphenylamino)-5-fluoropyrimidin-4-ylamino)-6-methylphenyl)methanesulfonamide;
N-(2-(5-fluoro-2-(3-isopropoxy-4,5-dimethoxyphenylamino)pyrimidin-4-ylamino)-6-methylphenyl)methanesulfonamide;
N-(2-(5-fluoro-2-(3-isobutoxy-4,5-dimethoxyphenylamino)pyrimidin-4-ylamino)-6-methylphenyl)methanesulfonamide;
N-(2-(2-(3-(cyclopropylmethoxy)-4,5-dimethoxyphenylamino)-5-fluoropyrimidin-4-ylamino)-6-methylphenyl)methanesulfonamide;
N-(2-(5-fluoro-2-(3-isopropoxyphenylamino)pyrimidin-4-ylamino)-6-methylphenyl)methanesulfonamide;
N-(2-(5-fluoro-2-(3-propoxyphenylamino)pyrimidin-4-ylamino)-6-methylphenyl)methanesulfonamide;
N-(2-(5-chloro-2-(3-(2-hydroxyethoxy)phenylamino)pyrimidin-4-ylamino)phenyl)methanesulfonamide;
N-(2-(5-chloro-2-(3-(2-(piperidin-1-yl)ethoxy)phenylamino)pyrimidin-4-ylamino)phenyl)methanesulfonamide;
N-(2-(5-chloro-2-(4-methoxy-3-(2-(piperidin-1-yl)ethoxy)phenylamino)pyrimidin-4-ylamino)phenyl)methanesulfonamide;
N-(2-(5-chloro-2-(3-(2-(diethylamino)ethoxy)phenylamino)pyrimidin-4-ylamino)phenyl)methanesulfonamide;
N-(2-(5-chloro-2-(4-(2-(diethylamino)ethoxy)phenylamino)pyrimidin-4-ylamino)phenyl)methanesulfonamide;
N-(2-(5-chloro-2-(3-(2-(piperidin-1-yl)ethoxy)phenylamino)pyrimidin-4-ylamino)-6-methylphenyl)methanesulfonamide;
N-(2-(5-chloro-2-(3-(piperidin-4-ylmethoxy)phenylamino)pyrimidin-4-ylamino)phenyl)methanesulfonamide;
N-(2-(5-chloro-2-(3-(2-(2-oxopyrrolidin-1-yl)ethoxy)phenylamino)pyrimidin-4-ylamino)phenyl)methanesulfonamide;
N-(2-(5-chloro-2-(3-(2-(pyrrolidin-2-yl)ethoxy)phenylamino)pyrimidin-4-ylamino)phenyl)methanesulfonamide;
N-(4-(5-chloro-4-(2-(methylsulfonamido)phenylamino)pyrimidin-2-ylamino)phenyl)-2-(pyrrolidin-1-yl)acetamide;
N-(2-(5-chloro-2-(3-(2-(piperazin-1-yl)ethoxy)phenylamino)pyrimidin-4-ylamino)phenyl)methanesulfonamide trifluoroacetate;
N-(2-(5-Chloro-2-(3-(3-piperidin-1-yl)propoxy)phenylamino)pyrimidin-4-ylamino)phenyl)methanesulfonamide;
N-(2-(5-chloro-2-(3-(3-methylpiperazin-1-yl)propoxy)phenylamino)pyrimidin-4-ylamino)phenyl)methanesulfonamide;
N-(2-(5-chloro-2-(4-(3-(4-methylpiperazin-1-yl)propoxy)phenylamino)pyrimidin-4-ylamino)phenyl)methanesulfonamide;
N-(2-(5-chloro-2-(3-isobutoxy-4,5-dimethoxyphenylamino)pyrimidin-4-ylamino)phenyl)methanesulfonamide;
N-(2-(5-chloro-2-(3,4,5-trimethoxyphenylamino)pyrimidin-4-ylamino)phenyl)methanesulfonamide hydrochloride;
N-(2-(5-chloro-2-(4-methoxyphenylamino)pyrimidin-4-ylamino)phenyl)methanesulfonamide hydrochloride;
N-(2-(5-chloro-2-(3-(2-(pyrrolidin-1-yl)ethoxy)phenylamino)pyrimidin-4-ylamino)phenyl)methanesulfonamide;
N-(2-(5-chloro-2-(3-methoxy-4-(2-(pyrrolidin-1-yl)ethoxy)phenylamino)pyrimidin-4-ylamino)phenyl)methanesulfonamide;
N-(2-(5-chloro-2-(3-methoxy-4-(2-(piperidin-1-yl)ethoxy)phenylamino)pyrimidin-4-ylamino)phenyl)methanesulfonamide hydrochloride;
Isopropyl 2-(4-(5-chloro-4-(2-(methylsulfonamido)phenylamino)pyrimidin-2-ylamino)-phenoxy)acetate hydrochloride;
Ethyl 2-(4-(5-fluoro-4-(2-(methylsulfonamido)phenylamino)pyrimidin-2-ylamino)phenoxy)acetate hydrochloride;
N-(2-(5-chloro-2-(3-((1-methylpiperidin-2-yl)methoxy)phenylamino)pyrimidin-4-ylamino)phenyl)methanesulfonamide;
N-(2-(5-chloro-2-(3-((1-methylpiperidin-3-yl)methoxy)phenylamino)pyrimidin-4-ylamino)phenyl)methanesulfonamide;
N-(2-(5-chloro-2-(3-((1,4-dimethylpiperazin-2-yl)methoxy)phenylamino)pyrimidin-4-ylamino)phenyl)methanesulfonamide;
2-(4-(5-chloro-4-(2-(methylsulfonamido)phenylamino)pyrimidin-2-ylamino)phenyl)acetic acid;
2-(3-(5-chloro-4-(2-(methylsulfonamido)phenylamino)pyrimidin-2-ylamino)phenoxy)acetic acid hydrochloride;
2-(3-(5-chloro-4-(2-(methylsulfonamido)phenylamino)pyrimidin-2-ylamino)phenoxy)-N,N-diethylacetamide;
2-(3-(5-chloro-4-(2-(methylsulfonamido)phenylamino)pyrimidin-2-ylamino)phenoxy)-N-ethylacetamide 2,2,2-trifluoroacetate;
N-(2-(5-chloro-2-(3,4,5-trimethoxyphenylamino)pyrimidin-4-ylamino)-5-methoxyphenyl)methanesulfonamide;
N-(2-(5-chloro-2-(3,4-dimethoxyphenylamino)pyrimidin-4-ylamino)-5-methoxyphenyl)methanesulfonamide;
N-ethyl-N-(2-(5-fluoro-2-(3,4,5-trimethoxyphenylamino)pyrimidin-4-ylamino)phenyl)methanesulfonamide;
N-(2-(2-(3,5-dimethylphenylamino)-5-fluoropyrimidin-4-ylamino)phenyl)-N-ethylmethanesulfonamide;
N-ethyl-N-(2-(5-fluoro-2-(4-methoxyphenylamino)pyrimidin-4-ylamino)phenyl)methanesulfonamide;
N-(2-(2-(3,4-dimethoxyphenylamino)-5-fluoropyrimidin-4-ylamino)phenyl)-N-ethylmethanesulfonamide;
N-ethyl-N-(2-(5-fluoro-2-(4-(2-(piperidin-1-yl)ethoxy)phenylamino)pyrimidin-4-ylamino)phenyl)methanesulfonamide;
N-ethyl-N-(2-(5-fluoro-2-(4-(2-morpholinoethoxy)phenylamino)pyrimidin-4-ylamino)phenyl)methanesulfonamide;
N-(2-(5-chloro-2-(3,4,5-trimethoxyphenylamino)pyrimidin-4-ylamino)-6-methylphenyl)methanesulfonamide;
N-(2-(5-fluoro-2-(3,4,5-trimethoxyphenylamino)pyrimidin-4-ylamino)-6-isopropylphenyl)methanesulfonamide;
N-(3-fluoro-6-(5-fluoro-2-(3,4,5-trimethoxyphenylamino)pyrimidin-4-ylamino)-2-methylphenyl)methanesulfonamide;
N-(6-(5-fluoro-2-(3,4,5-trimethoxyphenylamino)pyrimidin-4-ylamino)-3-methoxy-2-methylphenyl)methanesulfonamide;
N-(6-(5-chloro-2-(3,4,5-trimethoxyphenylamino)pyrimidin-4-ylamino)-2,3-dihydrobenzo[b][1,4]dioxin-5-yl)methanesulfonamide;
N-(6-(5-fluoro-2-(3,4,5-trimethoxyphenylamino)pyrimidin-4-ylamino)-2,3-dimethylphenyl)methanesulfonamide;
N-(2-ethoxy-6-(5-fluoro-2-(3,4,5-trimethoxyphenylamino)pyrimidin-4-ylamino)phenyl)methanesulfonamide;
N-(6-(5-fluoro-2-(3,4,5-trimethoxyphenylamino)pyrimidin-4-ylamino)-2,3-dihydrobenzo[b][1,4]dioxin-5-yl)methanesulfonamide;
N-(3-ethoxy-6-(5-fluoro-2-(3,4,5-trimethoxyphenylamino)pyrimidin-4-ylamino)-2-methylphenyl)methanesulfonamide;
N-(2-ethyl-6-(5-fluoro-2-(3,4,5-trimethoxyphenylamino)pyrimidin-4-ylamino)phenyl)methanesulfonamide;
N-(2-(5-Fluoro-2-(3,4,5-trimethoxyphenylamino)pyrimidin-4-ylamino)-5-methylphenyl)methanesulfonamide;
N-(2-(5-fluoro-2-(3,4,5-trimethoxyphenylamino)pyrimidin-4-ylamino)-5-morpholinophenyl)methanesulfonamide;
N-(2-(5-fluoro-2-(3,4,5-trimethoxyphenylamino)pyrimidin-4-ylamino)-6-methoxyphenyl)methanesulfonamide;
N-(2-(5-fluoro-2-(3,4,5-trimethoxyphenylamino)pyrimidin-4-ylamino)-5-methoxyphenyl)methanesulfonamide;
N-(4,5-difluoro-2-(5-fluoro-2-(3,4,5-trimethoxyphenylamino)pyrimidin-4-ylamino)phenyl)methanesulfonamide;
N-(5-ethoxy-2-(5-fluoro-2-(3,4,5-trimethoxyphenylamino)pyrimidin-4-ylamino)phenyl)methanesulfonamide;
N-(2-methyl-6-(2-(3,4,5-trimethoxyphenylamino)pyrimidin-4-ylamino)phenyl)methanesulfonamide;
N-(2-(2-(3-methoxy-5-(2-(piperidin-1-yl)ethoxy)phenylamino)pyrimidin-4-ylamino)-6-methylphenyl)methanesulfonamide formate salt;
N-(2-(5-bromo-2-(3,4,5-trimethoxyphenylamino)pyrimidin-4-ylamino)-5-methoxyphenyl)methanesulfonamide;
N-(2-(5-bromo-2-(3,4-dimethoxyphenylamino)pyrimidin-4-ylamino)-5-methoxyphenyl)methanesulfonamide;
N-(2-(2-(3,4-dimethoxyphenylamino)-5-fluoropyrimidin-4-ylamino)-5-methoxyphenyl)methanesulfonamide;
N-(2-(5-fluoro-2-(3,4,5-trimethoxyphenylamino)pyrimidin-4-ylamino)-6-methylphenyl)-N-methylmethanesulfonamide;
N-(2-(5-fluoro-2-(3,4,5-trimethoxyphenylamino)pyrimidin-4-ylamino)-5-isopropoxyphenyl)methanesulfonamide;
N-(2-fluoro-6-(5-fluoro-2-(3,4,5-trimethoxyphenylamino)pyrimidin-4-ylamino)phenyl)methanesulfonamide;
N-(2-chloro-6-(5-fluoro-2-(3,4,5-trimethoxyphenylamino)pyrimidin-4-ylamino)phenyl)methanesulfonamide;
N-(2-(5-fluoro-2-(3,4,5-trimethoxyphenylamino)pyrimidin-4-ylamino)-5-(3-(piperidin-1-yl)propoxy)phenyl)methanesulfonamide;
N-(2-(5-fluoro-2-(3,4,5-trimethoxyphenylamino)pyrimidin-4-ylamino)-4,6-dimethylphenyl)methanesulfonamide;
N-(6-(5-chloro-2-(3,4,5-trimethoxyphenylamino)pyrimidin-4-ylamino)-2-fluoro-3-methoxyphenyl)methanesulfonamide;
N-(2-(2-(3-(1H-tetrazol-1-yl)phenylamino)-5-fluoropyrimidin-4-ylamino)phenyl)methanesulfonamide;
N-(2-(5-fluoro-2-(3-(5-methyl-1,2,4-oxadiazol-3-yl)phenylamino)pyrimidin-4-ylamino)phenyl)methanesulfonamide;
N-(2-(2-(3-(1H-tetrazol-5-yl)phenylamino)-5-fluoropyrimidin-4-ylamino)phenyl)methanesulfonamide;
N-(2-(5-fluoro-2-(4-(3-methyl-1H-1,2,4-triazol-5-yl)phenylamino)pyrimidin-4-ylamino)phenyl)methanesulfonamide;
N-(2-(5-fluoro-2-(4-(2-methylthiazol-4-yl)phenylamino)pyrimidin-4-ylamino)phenyl)methanesulfonamide;
N-(2-(5-fluoro-2-(3-(oxazol-5-yl)phenylamino)pyrimidin-4-ylamino)phenyl)methanesulfonamide;
N-(2-(5-fluoro-2-(3-(5-methyl-1H-tetrazol-1-yl)phenylamino)pyrimidin-4-ylamino)phenyl)methanesulfonamide;
N-(2-(2-(4-(1H-tetrazol-1-yl)phenylamino)-5-fluoropyrimidin-4-ylamino)phenyl)methanesulfonamide;
N-(2-(2-(4-cyanophenylamino)-5-fluoropyrimidin-4-ylamino)phenyl)methanesulfonamide;
N-(2-(5-fluoro-2-(4-(5-methyl-1,2,4-oxadiazol-3-yl)phenylamino)pyrimidin-4-ylamino)phenyl)methanesulfonamide;
N-(2-(2-(4-(1H-1,2,4-triazol-1-yl)phenylamino)-5-fluoropyrimidin-4-ylamino)phenyl)methanesulfonamide;
N-(2-(5-fluoro-2-(4-(5-methyl-1H-tetrazol-1-yl)phenylamino)pyrimidin-4-ylamino)phenyl)methanesulfonamide;
N-(2-(2-(3-cyanophenylamino)-5-fluoropyrimidin-4-ylamino)phenyl)methanesulfonamide;
N-(2-(5-fluoro-2-(3-(1-methyl-1H-pyrazol-3-yl)phenylamino)pyrimidin-4-ylamino)phenyl)methane sulfonamide;
N-(2-(2-(3-(2,5-dimethyl-1H-pyrrol-1-yl)phenylamino)-5-fluoropyrimidin-4-ylamino)phenyl)methane sulfonamide;
N-(2-(2-(3-(1H-tetrazol-1-yl)phenylamino)-5-chloropyrimidin-4-ylamino)phenyl)methane sulfonamide;
N-(2-(5-chloro-2-(4-cyanophenylamino)pyrimidin-4-ylamino)phenyl)methane sulfonamide;
N-(2-(5-chloro-2-(3-cyanophenylamino)pyrimidin-4-ylamino)phenyl)methane sulfonamide;
N-(2-(2-(3-(1H-pyrazol-1-yl)phenylamino)-5-chloropyrimidin-4-ylamino)phenyl)methane sulfonamide;
N-(2-(2-(4-(1H-pyrazol-1-yl)phenylamino)-5-chloropyrimidin-4-ylamino)phenyl)methane sulfonamide;
N-(2-(5-chloro-2-(3-(5-methyl-1,2,4-oxadiazol-3-yl)phenylamino)pyrimidin-4-ylamino)phenyl)methane sulfonamide;
N-(2-(5-chloro-2-(3-(3,5-dimethyl-1H-pyrazol-1-yl)phenylamino)pyrimidin-4-ylamino)phenyl)methane sulfonamide;
N-(2-(5-chloro-2-(4-(3,5-dimethyl-1H-pyrazol-1-yl)phenylamino)pyrimidin-4-ylamino)phenyl)methane sulfonamide;
N-(2-(2-(3-(1H-1,2,4-triazol-1-yl)phenylamino)-5-chloropyrimidin-4-ylamino)phenyl)methanesulfonamide;
N-(2-(2-(4-(1H-1,2,4-triazol-1-yl)phenylamino)-5-chloropyrimidin-4-ylamino)phenyl)methanesulfonamide;
N-(2-(5-chloro-2-(3-(3-methyl-1H-1,2,4-triazol-5-yl)phenylamino)pyrimidin-4-ylamino)phenyl)methanesulfonamide;
N-(2-(2-(4-(1H-tetrazol-1-yl)phenylamino)-5-chloropyrimidin-4-ylamino)phenyl)methanesulfonamide;
N-(2-(5-bromo-2-(4-(piperidin-1-yl)phenylamino)pyrimidin-4-ylamino)phenyl)methanesulfonamide; and
N-(2-(2-(3-(1H-tetrazol-1-yl)phenylamino)-5-bromopyrimidin-4-ylamino)phenyl)methanesulfonamide.

25. A pharmaceutical composition comprising a compound or a pharmaceutically acceptable salt thereof of claim 1 together with a pharmaceutically acceptable carrier, optionally in combination with one or more other pharmaceutical compositions.

26. A pharmaceutical composition of claim 25, comprising one or more additional compounds or pharmaceutically acceptable salts thereof selected from the group consisting of compounds of claim 1 and not being the first compound; other ZAP-70 inhibitors, steroids, leukotriene antagonists, cyclosporine or rapamycin.

27. (canceled)

28. (canceled)

29. (canceled)

30. (canceled)

31. (canceled)

32. (canceled)

33. (canceled)

34. A method for treating, controlling, delaying or preventing in a mammalian patient in need of the treatment of one or more conditions selected from the group consisting of diseases and disorders associated with ZAP-70, wherein the method comprises the administration to said patient a therapeutically effective amount of a compound of claim 1 or a pharmaceutically acceptable salt thereof.

35. A method for treating, controlling, delaying or preventing in a mammalian patient in need of the treatment of one or more conditions selected from the group consisting of immunological, inflammatory, autoimmune, allergic disorders, and immunologically-mediated diseases, wherein the method comprises the administration to said patient a therapeutically effective amount of a compound of claim 1 or a pharmaceutically acceptable salt thereof.

36. A method of claim 35, wherein the disease is acute or chronic inflammation; rheumatoid arthritis; multiple sclerosis; psoriasis; Crohn's disease; ulcerative colitis; systemic lupus erythematosus; asthma; chronic obstructive pulmonary disease (COPD); allergic rhinitis; allograft transplant rejection; or graft-versus-host disease.

37. A method for the preparation of a compound of claim 1, comprising the steps of
(a) reacting a compound of formula (II) 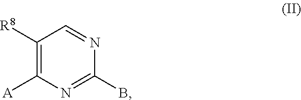
wherein R⁸ has the meaning as indicated in claim 1 and A, B are suitable leaving groups with one of the compounds of formula (III) or (IV) 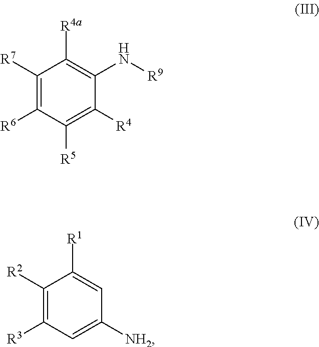
wherein R¹, R², R³, R⁴, R⁵, R⁶, R⁷, R⁹, R^{4a} have the meaning as indicated in claim 1 provided that one of R⁴, R⁵, R⁶, R⁷, R^{4a} is NHR^{24a}; or N(R^{24a})S(O)₂R²⁴, wherein R²⁴, R^{24a} have the meaning as indicated in claim 1;
(b) further reacting the resulting product (IIa) from step (a) with the other compound of formula (III) or (IV); and
when one of R⁴, R⁵, R⁶, R⁷, R^{4a} is NHR^{24a},
reacting the compound of formula (III) before step (a), product (IIa) after step (a) or the resulting product from step (b) with a compound of formula GS(O)₂NR²⁴, wherein G is a suitable leaving group to yield compounds of formula (I).
